(19) United States
(12) Patent Application Publication
(10) Pub. No.: US 2026/0062737 A1
(43) Pub. Date: Mar. 5, 2026

(54) SPATIAL BIOLOGY TOOLS USEFUL FOR DISEASE MONITORING

(71) Applicant: Singular Genomics Systems, Inc., San Diego, CA (US)

(72) Inventors: Eli N. GLEZER, Del Mar, CA (US); Michael LAWSON, Pasadena, CA (US); Kenneth Howard GOUIN, III, Oceanside, CA (US)

(21) Appl. No.: 19/318,081

(22) Filed: Sep. 3, 2025

Related U.S. Application Data
(60) Provisional application No. 63/690,740, filed on Sep. 4, 2024.

(51) Int. Cl. C12Q 1/6841; C12Q 1/6883

(52) U.S. Cl. CPC C12Q 1/6841; C12Q 1/6883; C12Q 2600/158

(57) ABSTRACT

Disclosed herein, inter alia, are oligonucleotide probes, methods, and kits useful for amplifying and detecting target nucleic acids in situ.

### CROSS-REFERENCES TO RELATED APPLICATIONS

This application claims the benefit of U.S. Provisional Application No. 63/690,740, filed Sep. 4, 2024, which is incorporated herein by reference in its entirety and for all purposes.

### SEQUENCE LISTING

The instant application contains a Sequence Listing which has been submitted electronically in XML format and is hereby incorporated by reference in its entirety. Said XML copy, created on Aug. 22, 2025, is named 051385-627001US.xml, and is 9,380,734 bytes in size.

### BACKGROUND

Inflammatory bowel disease (IBD) is a chronic autoimmune disorder, with ulcerative colitis (UC) and Crohn's disease (CD) representing the main IBD subtypes. Each malady is characterized by distinct patterns of chronic GI tract inflammation, with UC being primarily restricted to the colon and conferring an increased lifetime risk of colorectal cancer and Crohn's manifesting throughout the gut and frequently complicated by fistulas. Both diseases markedly impact quality of life. Disease flairs and resulting hospitalizations for complications, be they infectious or requiring of surgery, are frustratingly unpredictable. IBD is not static over time and varies through the GI tract. Although extensive endoscopic sampling (both spatially and across time) is part of routine clinical care, the low throughput, and thus high cost nature of existing platforms has made them simply impractical for use in large, well powered IBD clinical studies. This challenge has been compounded by the fact that most existing platforms track only one type of analyte (i.e. protein or transcript, thus increasing cost and sample requirements for being able to investigate both) and are not easily adapted to modest plexicity disease-focused applications. Users are often stuck using large, costly panels where most targets are irrelevant to the use-case. The high cost, low throughput, inflexible and awkward multi-platform nature of current spatial multi-omic technologies represent major barriers to the large scale translational studies needed to advance therapy development and patient care in IBD. Disclosed herein, inter alia, are solutions to these and other problems in the art.

### BRIEF SUMMARY

Described herein are aspects pertaining to methods of monitoring, detecting, and diagnosing a disease state, such as IBD, of a subject. Additional embodiments include methods and novel sequences useful for detecting IBD-associated biomolecules and in situ.

### BRIEF DESCRIPTION OF THE DRAWINGS

FIG. 1. Schematic of three different embodiments for detecting targets. Top: transcript counting; middle: direct-seq de novo RNA sequencing; bottom: protein detection.

FIG. 2A-2D. Spatial multi-omics data of a single 4×4 mm section of normal human tonsil. FIG. 2A. a fH&E™ image of the tonsil. FIG. 2B. An image of the tonsil section wherein 10 different proteins are detected. FIG. 2C. An image of the tonsil section wherein 153 different genes are detected. FIG. 2D. Detected transcripts overlaid on tissue section and differentiated by cell type classification.

FIG. 3. Schematic representation of a single padlock probe targeting TCRB RNA. Complementary sequences to the 3′-end of the FR3 and the 5′-end of the FR4 regions are indicated on the left and right, respectively. Reverse transcription from the 3′ end generates a copy of the CDR3 region. The asterisk indicates the ligation site. A primer, shown as an arrow in the upper right, may be used for initiating amplification. The 3′ end of the padlock probe (i.e., the sequence complementary to the FR4 region) may be used as a sequencing primer binding sequence. The T-Cell Receptor Beta (TCRB) RNA molecule is an essential component of the T-cell receptor (TCR) complex in the immune system. TCRs are pivotal for recognizing antigens presented by major histocompatibility complex (MHC) molecules on antigen-presenting cells. The TCR structure comprises various regions, including variable (V), diversity (D), joining (J), and constant (C) segments. Within the TCRB chain, specifically in its variable region, are the Framework 3 (FR3) and Framework 4 (FR4) regions. These framework regions are crucial in maintaining the structure and stability of the TCR. The FR3 region, located between the second and third complementarity-determining regions (CDR2 and CDR3), serves as a structural scaffold for the CDRs, which are directly involved in the antigen recognition process. This framework region contributes to the shape and orientation of the antigen-binding site. On the other hand, the FR4 region follows the CDR3 and is a part of the variable domain. FR4 plays a vital role in ensuring the structural integrity of the TCR.

FIGS. 4A-4C. G4X spatial multi-omics data of a single 4 mm punch of normal human colon. FIG. 4A. is the fH&E image, FIG. 4B. is the transcripts detected using a 155-plex colon specific panel. FIG. 4C. Image of the transcript-based cell type classification overlayed on fH&E.

### DETAILED DESCRIPTION

The aspects and embodiments described herein relate to systems and methods for analyzing a cell and cellular components (e.g., RNA transcripts, proteins, and/or biomolecule of a cell or tissue). Data obtained from the proteome and transcriptome is used in research to gain insight into processes such as cellular differentiation, carcinogenesis, transcription regulation, and biomarker discovery, among others. The methods provide significant advantages in terms of speed and detection efficiency of target polynucleotides, and may be performed on solid supports or in or on cells or tissue sections in situ.

### I. Definitions

All patents, patent applications, articles and publications mentioned herein, both supra and infra, are hereby expressly incorporated herein by reference in their entireties. The practice of the technology described herein will employ, unless indicated specifically to the contrary, conventional methods of chemistry, biochemistry, organic chemistry, molecular biology, bioinformatics, microbiology, recombinant DNA techniques, genetics, immunology, and cell biology that are within the skill of the art, many of which are described below for the purpose of illustration. Examples of such techniques are available in the literature. See, e.g., Singleton et al., DICTIONARY OF MICROBIOLOGY AND MOLECULAR BIOLOGY 2nd ed., J. Wiley & Sons (New York, NY 1994); and Sambrook and Green, Molecular Cloning: A Laboratory Manual, 4th Edition (2012). Methods, devices and materials similar or equivalent to those described herein can be used in the practice of this invention.

Unless defined otherwise herein, all technical and scientific terms used herein have the same meaning as commonly understood by one of ordinary skill in the art to which this disclosure belongs. Various scientific dictionaries that include the terms included herein are well known and available to those in the art. Although any methods and materials similar or equivalent to those described herein find use in the practice or testing of the disclosure, some preferred methods and materials are described. Accordingly, the terms defined immediately below are more fully described by reference to the specification as a whole. It is to be understood that this disclosure is not limited to the particular methodology, protocols, and reagents described, as these may vary, depending upon the context in which they are used by those of skill in the art. The following definitions are provided to facilitate understanding of certain terms used frequently herein and are not meant to limit the scope of the present disclosure.

As used herein, the singular terms “a”, “an”, and “the” include the plural reference unless the context clearly indicates otherwise. Reference throughout this specification to, for example, “one embodiment”, “an embodiment”, “another embodiment”, “a particular embodiment”, “a related embodiment”, “a certain embodiment”, “an additional embodiment”, or “a further embodiment” or combinations thereof means that a particular feature, structure or characteristic described in connection with the embodiment is included in at least one embodiment of the present disclosure. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the term “about” means a range of values including the specified value, which a person of ordinary skill in the art would consider reasonably similar to the specified value. In embodiments, the term “about” means within a standard deviation using measurements generally acceptable in the art. In embodiments, about means a range extending to +/−10% of the specified value. In embodiments, about means the specified value.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

As used herein, the term “control” or “control experiment” is used in accordance with its plain and ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects.

As used herein, the term “complement” is used in accordance with its plain and ordinary meaning and refers to a nucleotide (e.g., RNA nucleotide or DNA nucleotide) or a sequence of nucleotides capable of base pairing with a complementary nucleotide or sequence of nucleotides (e.g., Watson-Crick base pairing). As described herein and commonly known in the art the complementary (matching) nucleotide of adenosine is thymidine and the complementary (matching) nucleotide of guanosine is cytosine. Thus, a complement may include a sequence of nucleotides that base paired with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence. Another example of complementary sequences are a template sequence and an amplicon sequence polymerized by a polymerase along the template sequence. “Duplex” means at least two oligonucleotides and/or polynucleotides that are fully or partially complementary undergo Watson-Crick type base pairing among all or most of their nucleotides so that a stable complex is formed. Complementary single stranded nucleic acids and/or substantially complementary single stranded nucleic acids can hybridize to each other under hybridization conditions, thereby forming a nucleic acid that is partially or fully double stranded. When referring to a double-stranded polynucleotide including a first strand hybridized to a second strand, it is understood that each of the first strand and the second strand are independently single-stranded polynucleotides. All or a portion of a nucleic acid sequence may be substantially complementary to another nucleic acid sequence, in some embodiments. As referred to herein, “substantially complementary” refers to nucleotide sequences that can hybridize with each other under suitable hybridization conditions. Hybridization conditions can be altered to tolerate varying amounts of sequence mismatch within complementary nucleic acids that are substantially complementary. Substantially complementary portions of nucleic acids that can hybridize to each other can be 75% or more, 76% or more, 77% or more, 78% or more, 79% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more or 99% or more complementary to each other. In some embodiments substantially complementary portions of nucleic acids that can hybridize to each other are 100% complementary. Nucleic acids, or portions thereof, that are configured to hybridize to each other often include nucleic acid sequences that are substantially complementary to each other.

As described herein, the complementarity of sequences may be partial, in which only some of the nucleic acids match according to base pairing, or complete, where all the nucleic acids match according to base pairing. Thus, two sequences that are complementary to each other, may have a specified percentage of nucleotides that complement one another (e.g., about 60%, preferably 65%, 70%, 75%, 80%, 85%, 90%, 91%, 92%, 93%, 94%, 95%, 96%, 97%, 98%, 99%, or higher complementarity over a specified region). In embodiments, two sequences are complementary when they are completely complementary, having 100% complementarity. In embodiments, sequences in a pair of complementary sequences form portions of a single polynucleotide with non-base-pairing nucleotides (e.g., as in a hairpin or loop structure, with or without an overhang) or portions of separate polynucleotides. In embodiments, one or both sequences in a pair of complementary sequences form portions of longer polynucleotides, which may or may not include additional regions of complementarity.

As used herein, the term “contacting” is used in accordance with its plain ordinary meaning and refers to the process of allowing at least two distinct species (e.g., chemical compounds including biomolecules, particles, solid supports, or cells) to become sufficiently proximal to react, interact or physically touch. It should be appreciated, however, that the resulting reaction product can be produced directly from a reaction between the added reagents or from an intermediate from one or more of the added reagents which can be produced in the reaction mixture. The term “contacting” may include allowing two species to react, interact, or physically touch, wherein the two species may be a compound as described herein and a protein or enzyme.

As may be used herein, the terms “nucleic acid,” “nucleic acid molecule,” “nucleic acid sequence,” “strand,” “nucleic acid fragment” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may include natural nucleic acid sequences and variants thereof, artificial nucleic acid sequences, or a combination of such sequences. As may be used herein, the terms “nucleic acid oligomer” and “oligonucleotide” are used interchangeably and are intended to include, but are not limited to, nucleic acids having a length of 200 nucleotides or less. In some embodiments, an oligonucleotide is a nucleic acid having a length of 2 to 200 nucleotides, 2 to 150 nucleotides, 5 to 150 nucleotides or 5 to 100 nucleotides. The terms “polynucleotide,” “oligonucleotide,” “oligo” or the like refer, in the usual and customary sense, to a linear sequence of nucleotides. Oligonucleotides are typically from about 5, 6, 7, 8, 9, 10, 12, 15, 25, 30, 40, 50 or more nucleotides in length, up to about 100 nucleotides in length. In some embodiments, an oligonucleotide is a primer configured for extension by a polymerase when the primer is annealed completely or partially to a complementary nucleic acid template. A primer is often a single stranded nucleic acid. In embodiments, a primer, or portion thereof, is substantially complementary to a portion of an adapter. In some embodiments, a primer has a length of 200 nucleotides or less. In embodiments, a primer has a length of 10 to 150 nucleotides, 15 to 150 nucleotides, 5 to 100 nucleotides, 5 to 50 nucleotides or 10 to 50 nucleotides. In some embodiments, an oligonucleotide may be immobilized to a solid support. In some embodiments, a polynucleotide may be a circular polynucleotide. The terms “circular polynucleotide” or “circular oligonucleotide” refer to a contiguous polynucleotide lacking a free 5′ and a free 3′ end.

As used herein, the terms “polynucleotide primer” and “primer” refers to any polynucleotide molecule that may hybridize to a polynucleotide template, be bound by a polymerase, and be extended in a template-directed process for nucleic acid synthesis (e.g., amplification and/or sequencing). The primer may be a separate polynucleotide from the polynucleotide template, or both may be portions of the same polynucleotide (e.g., as in a hairpin structure having a 3′ end that is extended along another portion of the polynucleotide to extend a double-stranded portion of the hairpin). Primers (e.g., forward or reverse primers) may be attached to a solid support. A primer can be of any length depending on the particular technique it will be used for. For example, PCR primers are generally between 10 and 40 nucleotides in length. The length and complexity of the nucleic acid fixed onto the nucleic acid template may vary. In some embodiments, a primer has a length of 200 nucleotides or less. In embodiments, a primer has a length of 10 to 150 nucleotides, 15 to 150 nucleotides, 5 to 100 nucleotides, 5 to 50 nucleotides or 10 to 50 nucleotides. In embodiments, a primer has a length of 10 to 150 nucleotides, 15 to 150 nucleotides, 5 to 100 nucleotides, 5 to 50 nucleotides or 10 to 50 nucleotides. A primer typically has a length of 10 to 50 nucleotides. For example, a primer may have a length of 10 to 40, 10 to 30, 10 to 20, 25 to 50, 15 to 40, 15 to 30, 20 to 50, 20 to 40, or 20 to 30 nucleotides. In some embodiments, a primer has a length of 18 to 24 nucleotides. One of skill can adjust these factors to provide optimum hybridization and signal production for a given hybridization procedure. The primer permits the addition of a nucleotide residue thereto, or oligonucleotide or polynucleotide synthesis therefrom, under suitable conditions. In an embodiment the primer is a DNA primer, i.e., a primer consisting of, or largely consisting of, deoxyribonucleotide residues. The primers are designed to have a sequence that is the complement of a region of template/target DNA to which the primer hybridizes. The addition of a nucleotide residue to the 3′ end of a primer by formation of a phosphodiester bond results in a DNA extension product. The addition of a nucleotide residue to the 3′ end of the DNA extension product by formation of a phosphodiester bond results in a further DNA extension product. In another embodiment the primer is an RNA primer. In embodiments, a primer is hybridized to a target polynucleotide. A “primer” is complementary to a polynucleotide template, and complexes by hydrogen bonding or hybridization with the template to give a primer/template complex for initiation of synthesis by a polymerase, which is extended by the addition of covalently bonded bases linked at its 3′ end complementary to the template in the process of DNA synthesis. A “splint oligonucleotide” is used in accordance with its plain and ordinary meaning and refers to an oligonucleotide having 2 or more sequences complementary to two or more portions of a polynucleotide. An “oligonucleotide probe” or “oligonucleotide primer”, as used herein, refers to a primer including a sequence (e.g., a target hybridization sequence) at a 3′ end complementary to a sequence (e.g., a probe hybridization sequence) of a target polynucleotide (e.g., a target mRNA molecule). In embodiments, the oligonucleotide probe includes one or more sequences located 5′ (i.e., upstream) of the target hybridization sequence, for example, one or more primer binding sequences. An “extended oligonucleotide probe” or “extended oligonucleotide primer”, as used herein, refers to an oligonucleotide probe that has had one or more nucleotides incorporated into the 3′ end by a polymerase, for example, a reverse transcriptase. In embodiments, an extended oligonucleotide probe includes a region of cDNA (e.g., a cDNA sequence complementary to a portion of an mRNA molecule) located 3′ (i.e., downstream) of the target hybridization sequence. A “target hybridization sequence” as used herein refers to a sequence at a 3′ end of an oligonucleotide probe that is complementary to a sequence in a target polynucleotide (e.g., complementary to a probe hybridization sequence of the target polynucleotide).

As used herein, the term “primer binding sequence” refers to a polynucleotide sequence that is complementary to at least a portion of a primer (e.g., a sequencing primer or an amplification primer). Primer binding sequences can be of any suitable length. In embodiments, a primer binding sequence is about or at least about 10, 15, 20, 25, 30, or more nucleotides in length. In embodiments, a primer binding sequence is 10-50, 15-30, or 20-25 nucleotides in length. The primer binding sequence may be selected such that the primer (e.g., sequencing primer) has the preferred characteristics to minimize secondary structure formation or minimize non-specific amplification, for example having a length of about 20-30 nucleotides; approximately 50% GC content, and a Tm of about 55° C. to about 65° C.

Nucleic acids, including e.g., nucleic acids with a phosphorothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent or other interaction.

The order of elements within a nucleic acid molecule is typically described herein from 5′ to 3′. In the case of a double-stranded molecule, the “top” strand is typically shown from 5′ to 3′, according to convention, and the order of elements is described herein with reference to the top strand.

The term “messenger RNA” or “mRNA” refers to an RNA that is without introns and is capable of being translated into a polypeptide. The term “RNA” refers to any ribonucleic acid, including but not limited to mRNA, tRNA (transfer RNA), rRNA (ribosomal RNA), and/or noncoding RNA (such as lncRNA (long noncoding RNA)). The term “cDNA” refers to a DNA that is complementary or identical to an RNA, in either single stranded or double stranded form.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

As used herein, the term “associated” or “associated with” can mean that two or more species are identifiable as being co-located at a point in time. An association can mean that two or more species are or were within a similar container. An association can be an informatics association, where for example digital information regarding two or more species is stored and can be used to determine that one or more of the species were co-located at a point in time. An association can also be a physical association. In some instances two or more associated species are “tethered”, “coated”, “attached”, or “immobilized” to one another or to a common solid or semisolid support (e.g. a receiving substrate). An association may refer to a relationship, or connection, between two entities. For example, a barcode sequence may be associated with a particular target by binding a probe including the barcode sequence to the target. In embodiments, detecting the associated barcode provides detection of the target. Associated may refer to the relationship between a sample and the DNA molecules, RNA molecules, or polynucleotides originating from or derived from that sample. These relationships may be encoded in oligonucleotide barcodes, as described herein. A polynucleotide is associated with a sample if it is an endogenous polynucleotide, i.e., it occurs in the sample at the time the sample is obtained, or is derived from an endogenous polynucleotide. For example, the RNAs endogenous to a cell are associated with that cell. cDNAs resulting from reverse transcription of these RNAs, and DNA amplicons resulting from PCR amplification of the cDNAs, contain the sequences of the RNAs and are also associated with the cell. The polynucleotides associated with a sample need not be located or synthesized in the sample, and are considered associated with the sample even after the sample has been destroyed (for example, after a cell has been lysed). Barcoding can be used to determine which polynucleotides in a mixture are associated with a particular sample. In embodiments, a proximity probe is associated with a particular barcode, such that identifying the barcode identifies the probe with which it is associated. Because the proximity probe specifically binds to a target, identifying the barcode thus identifies the target.

As used herein, the term “IBD-associated gene” refers to a nucleic acid sequence, including DNA, RNA, cDNA, or an amplification product thereof, that is a priori known to be associated with inflammatory bowel disease in a biological, analytical, or informatic context. Consistent with the definition of “associated” provided herein, the relationship between a gene and IBD is understood before detection, and may be established through its biological origin, its physical interaction with a probe, its representation in curated datasets, and/or its spatial localization within a cell or tissue has been correlated with disease pathology.

The term “adapter” as used herein refers to any oligonucleotide that can be ligated to a nucleic acid molecule, thereby generating nucleic acid products that can be sequenced on a sequencing platform (e.g., an Illumina or Singular Genomics G4™ sequencing platform). In embodiments, adapters include two reverse complementary oligonucleotides forming a double-stranded structure. In embodiments, an adapter includes two oligonucleotides that are complementary at one portion and mismatched at another portion, forming a Y-shaped or fork-shaped adapter that is double stranded at the complementary portion and has two overhangs at the mismatched portion. Since Y-shaped adapters have a complementary, double-stranded region, they can be considered a special form of double-stranded adapters. When this disclosure contrasts Y-shaped adapters and double stranded adapters, the term “double-stranded adapter” or “blunt-ended” is used to refer to an adapter having two strands that are fully complementary, substantially (e.g., more than 90% or 95%) complementary, or partially complementary. In embodiments, adapters include sequences that bind to sequencing primers. In embodiments, adapters include sequences that bind to immobilized oligonucleotides (e.g., P7 and P5 sequences) or reverse complements thereof. In embodiments, the adapter is substantially non-complementary to the 3′ end or the 5′ end of any target polynucleotide present in the sample. In embodiments, the adapter can include a sequence that is substantially identical, or substantially complementary, to at least a portion of a primer, for example a universal primer. In embodiments, the adapter can include an index sequence (also referred to as barcode or tag) to assist with downstream error correction, identification or sequencing.

As used herein, the terms “analogue” and “analog”, in reference to a chemical compound, refers to compound having a structure similar to that of another one, but differing from it in respect of one or more different atoms, functional groups, or substructures that are replaced with one or more other atoms, functional groups, or substructures. In the context of a nucleotide, a nucleotide analog refers to a compound that, like the nucleotide of which it is an analog, can be incorporated into a nucleic acid molecule (e.g., an extension product) by a suitable polymerase, for example, a DNA polymerase in the context of a nucleotide analogue. The terms also encompass nucleic acids containing known nucleotide analogs or modified backbone residues or linkages, which are synthetic, naturally occurring, or non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphorothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see, e.g., see Eckstein, OLIGONUCLEOTIDES AND ANALOGUES: A PRACTICAL APPROACH, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA)), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

Other analog nucleic acids include bis-locked nucleic acids (bisLNAs; e.g., including those described in Moreno P M D et al. Nucleic Acids Res. 2013; 41(5):3257-73), twisted intercalating nucleic acids (TINAs; e.g., including those described in Doluca O et al. Chembiochem. 2011; 12(15):2365-74), bridged nucleic acids (BNAs; e.g., including those described in Soler-Bistue A et al. Molecules. 2019; 24(12): 2297), 2′-O-methyl RNA:DNA chimeric nucleic acids (e.g., including those described in Wang S and Kool E T. Nucleic Acids Res. 1995; 23(7):1157-1164), minor groove binder (MGB) nucleic acids (e.g., including those described in Kutyavin I V et al. Nucleic Acids Res. 2000; 28(2):655-61), morpholino nucleic acids (e.g., including those described in Summerton J and Weller D. Antisense Nucleic Acid Drug Dev. 1997; 7(3):187-95), C5-modified pyrimidine nucleic acids (e.g., including those described in Kumar P et al. J. Org. Chem. 2014; 79(11): 5047-5061), peptide nucleic acids (PNAs; e.g., including those described in Gupta A et al. J. Biotechnol. 2017; 259: 148-59), and/or phosphorothioate nucleotides (e.g., including those described in Eckstein F. Nucleic Acid Ther. 2014; 24(6):374-87).

As used herein, a “native” nucleotide is used in accordance with its plain and ordinary meaning and refers to a naturally occurring nucleotide that does not include an exogenous label (e.g., a fluorescent dye, or other label) or chemical modification such as may characterize a nucleotide analog. Examples of native nucleotides useful for carrying out procedures described herein include: dATP (2′-deoxyadenosine-5′-triphosphate); dGTP (2′-deoxyguanosine-5′-triphosphate); dCTP (2′-deoxycytidine-5′-triphosphate); dTTP (2′-deoxythymidine-5′-triphosphate); and dUTP (2′-deoxyuridine-5′-triphosphate).

In embodiments, the nucleotides of the present disclosure use a cleavable linker to attach the label to the nucleotide. The use of a cleavable linker ensures that the label can, if required, be removed after detection, avoiding any interfering signal with any labelled nucleotide incorporated subsequently. The use of the term “cleavable linker” is not meant to imply that the whole linker is required to be removed from the nucleotide base. The cleavage site can be located at a position on the linker that ensures that part of the linker remains attached to the nucleotide base after cleavage. The linker can be attached at any position on the nucleotide base provided that Watson-Crick base pairing can still be carried out. In the context of purine bases, it is preferred if the linker is attached via the 7-position of the purine or the preferred deazapurine analogue, via an 8-modified purine, via an N-6 modified adenosine or an N-2 modified guanine. For pyrimidines, attachment is preferably via the 5-position on cytidine, thymidine or uracil and the N-4 position on cytosine.

The term “cleavable linker” or “cleavable moiety” as used herein refers to a divalent or monovalent, respectively, moiety which is capable of being separated (e.g., detached, split, disconnected, hydrolyzed, a stable bond within the moiety is broken) into distinct entities. A cleavable linker is cleavable (e.g., specifically cleavable) in response to external stimuli (e.g., enzymes, nucleophilic/basic reagents, reducing agents, photo-irradiation, electrophilic/acidic reagents, organometallic and metal reagents, or oxidizing reagents). A chemically cleavable linker refers to a linker which is capable of being split in response to the presence of a chemical (e.g., acid, base, oxidizing agent, reducing agent, Pd(0), tris-(2-carboxyethyl)phosphine, dilute nitrous acid, fluoride, tris(3-hydroxypropyl)phosphine), sodium dithionite (Na₂S₂O₄), or hydrazine (N₂H₄)). A chemically cleavable linker is non-enzymatically cleavable. In embodiments, the cleavable linker is cleaved by contacting the cleavable linker with a cleaving agent. In embodiments, the cleaving agent is a phosphine containing reagent (e.g., TCEP or THPP), sodium dithionite (Na₂S₂O₄), weak acid, hydrazine (N₂H₄), Pd(O), or light-irradiation (e.g., ultraviolet radiation). In embodiments, cleaving includes removing. A “cleavable site” or “scissile linkage” in the context of a polynucleotide is a site which allows controlled cleavage of the polynucleotide strand (e.g., the linker, the primer, or the polynucleotide) by chemical, enzymatic, or photochemical means known in the art and described herein. A scissile site may refer to the linkage of a nucleotide between two other nucleotides in a nucleotide strand (i.e., an internucleosidic linkage). In embodiments, the scissile linkage can be located at any position within the one or more nucleic acid molecules, including at or near a terminal end (e.g., the 3′ end of an oligonucleotide) or in an interior portion of the one or more nucleic acid molecules. In embodiments, conditions suitable for separating a scissile linkage include a modulating the pH and/or the temperature. In embodiments, a scissile site can include at least one acid-labile linkage. For example, an acid-labile linkage may include a phosphoramidate linkage. In embodiments, a phosphoramidate linkage can be hydrolysable under acidic conditions, including mild acidic conditions such as trifluoroacetic acid and a suitable temperature (e.g., 30° C.), or other conditions known in the art, for example Matthias Mag, et al Tetrahedron Letters, Volume 33, Issue 48, 1992, 7319-7322. In embodiments, the scissile site can include at least one photolabile internucleosidic linkage (e.g., o-nitrobenzyl linkages, as described in Walker et al, J. Am. Chem. Soc. 1988, 110, 21, 7170-7177), such as o-nitrobenzyloxymethyl or p-nitrobenzyloxymethyl group(s). In embodiments, the scissile site includes at least one uracil nucleobase. In embodiments, a uracil nucleobase can be cleaved with a uracil DNA glycosylase (UDG) or Formamidopyrimidine DNA Glycosylase Fpg. In embodiments, the scissile linkage site includes a sequence-specific nicking site having a nucleotide sequence that is recognized and nicked by a nicking endonuclease enzyme or a uracil DNA glycosylase. Cleavage agents used in methods described herein may be selected from nicking endonucleases, DNA glycosylases, or any single-stranded cleavage agents described in further detail elsewhere herein. Enzymes for cleavage of single-stranded DNA may be used for cleaving heteroduplexes in the vicinity of mismatched bases, D-loops, heteroduplexes formed between two strands of DNA which differ by a single base, an insertion or deletion. Mismatch recognition proteins that cleave one strand of the mismatched DNA in the vicinity of the mismatch site may be used as cleavage agents. Nonenzymatic cleaving may also be done through photodegredation of a linker introduced through a custom oligonucleotide used in a PCR reaction.

As used herein, the term “modified nucleotide” refers to nucleotide modified in some manner. Typically, a nucleotide contains a single 5-carbon sugar moiety, a single nitrogenous base moiety and 1 to three phosphate moieties. In embodiments, a nucleotide can include a blocking moiety and/or a label moiety. A blocking moiety on a nucleotide prevents formation of a covalent bond between the 3′ hydroxyl moiety of the nucleotide and the 5′ phosphate of another nucleotide. A blocking moiety on a nucleotide can be reversible, whereby the blocking moiety can be removed or modified to allow the 3′ hydroxyl to form a covalent bond with the 5′ phosphate of another nucleotide. A blocking moiety can be effectively irreversible under particular conditions used in a method set forth herein. In embodiments, the blocking moiety is attached to the 3′ oxygen of the nucleotide and is independently —NH₂, —CN, —CH₃, C₂-C₆ allyl (e.g., —CH₂—CH═CH₂), methoxyalkyl (e.g., —CH₂—O—CH₃), or —CH₂N₃. In embodiments, the blocking moiety is attached to the 3′ oxygen of the nucleotide and is independently

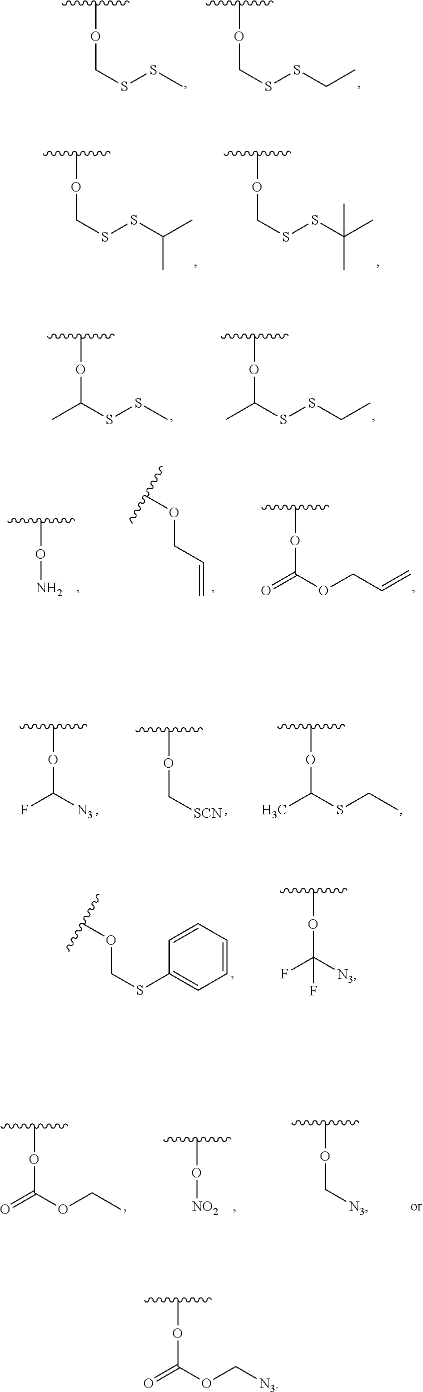

A label moiety of a modified nucleotide can be any moiety that allows the nucleotide to be detected, for example, using a spectroscopic method. Exemplary label moieties are fluorescent labels, mass labels, chemiluminescent labels, electrochemical labels, detectable labels and the like. One or more of the above moieties can be absent from a nucleotide used in the methods and compositions set forth herein. For example, a nucleotide can lack a label moiety or a blocking moiety or both. Examples of nucleotide analogues include, without limitation, 7-deaza-adenine, 7-deaza-guanine, the analogues of deoxynucleotides shown herein, analogues in which a label is attached through a cleavable linker to the 5-position of cytosine or thymine or to the 7-position of deaza-adenine or deaza-guanine, and analogues in which a small chemical moiety is used to cap the OH group at the 3′-position of deoxyribose. Nucleotide analogues and DNA polymerase-based DNA sequencing are also described in U.S. Pat. No. 6,664,079, which is incorporated herein by reference in its entirety for all purposes. Non-limiting examples of detectable labels include labels including fluorescent dyes, biotin, digoxin, haptens, and epitopes. In general, a dye is a molecule, compound, or substance that can provide an optically detectable signal, such as a colorimetric, luminescent, bioluminescent, chemiluminescent, phosphorescent, or fluorescent signal. In embodiments, the dye is a fluorescent dye. Non-limiting examples of dyes, some of which are commercially available, include CF dyes (Biotium, Inc.), Alexa Fluor dyes (Thermo Fisher), DyLight dyes (Thermo Fisher), Cy dyes (GE Healthscience), IRDyes (Li-Cor Biosciences, Inc.), and HiLyte dyes (Anaspec, Inc.). In embodiments, the label is a fluorophore.

In some embodiments, a nucleic acid includes a label. As used herein, the term “label” or “labels” is used in accordance with their plain and ordinary meanings and refer to molecules that can directly or indirectly produce or result in a detectable signal either by themselves or upon interaction with another molecule. Non-limiting examples of detectable labels include fluorescent dyes, biotin, digoxin, haptens, and epitopes. In general, a dye is a molecule, compound, or substance that can provide an optically detectable signal, such as a colorimetric, luminescent, bioluminescent, chemiluminescent, phosphorescent, or fluorescent signal. In embodiments, the label is a dye. In embodiments, the dye is a fluorescent dye. Non-limiting examples of dyes, some of which are commercially available, include CF dyes (Biotium, Inc.), Alexa Fluor dyes (Thermo Fisher), DyLight dyes (Thermo Fisher), Cy dyes (GE Healthscience), IRDyes (Li-Cor Biosciences, Inc.), and HiLyte dyes (Anaspec, Inc.). In embodiments, a particular nucleotide type is associated with a particular label, such that identifying the label identifies the nucleotide with which it is associated. In embodiments, the label is luciferin that reacts with luciferase to produce a detectable signal in response to one or more bases being incorporated into an elongated complementary strand, such as in pyrosequencing. In embodiment, a nucleotide includes a label (such as a dye). In embodiments, the label is not associated with any particular nucleotide, but detection of the label identifies whether one or more nucleotides having a known identity were added during an extension step (such as in the case of pyrosequencing). Examples of detectable agents (i.e., labels) include imaging agents, including fluorescent and luminescent substances, molecules, or compositions, including, but not limited to, a variety of organic or inorganic small molecules commonly referred to as “dyes,” “labels,” or “indicators.” Examples include fluorescein, rhodamine, acridine dyes, Alexa dyes, and cyanine dyes. In embodiments, the detectable moiety is a fluorescent molecule (e.g., acridine dye, cyanine, dye, fluorine dye, oxazine dye, phenanthridine dye, or rhodamine dye). In embodiments, the detectable moiety is a fluorescent molecule (e.g., acridine dye, cyanine, dye, fluorine dye, oxazine dye, phenanthridine dye, or rhodamine dye). The term “cyanine” or “cyanine moiety” as described herein refers to a detectable moiety containing two nitrogen groups separated by a polymethine chain. In embodiments, the cyanine moiety has 3 methine structures (i.e., cyanine 3 or Cy3). In embodiments, the cyanine moiety has 5 methine structures (i.e., cyanine 5 or Cy5). In embodiments, the cyanine moiety has 7 methine structures (i.e., cyanine 7 or Cy7).

The term “nucleoside” refers, in the usual and customary sense, to a glycosylamine including a nucleobase and a five-carbon sugar (ribose or deoxyribose). Non-limiting examples of nucleosides include cytidine, uridine, adenosine, guanosine, thymidine and inosine. Nucleosides may be modified at the base and/or the sugar. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g., polynucleotides contemplated herein include any types of RNA, e.g., mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site www.ncbi.nlm.nih.gov/BLAST/or the like). Such sequences are then said to be “substantially identical.” This definition also refers to, or may be applied to, the complement of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

As used herein, the term “removable” group, e.g., a label or a blocking group or protecting group, is used in accordance with its plain and ordinary meaning and refers to a chemical group that can be removed from a nucleotide analogue such that a DNA polymerase can extend the nucleic acid (e.g., a primer or extension product) by the incorporation of at least one additional nucleotide. Removal may be by any suitable method, including enzymatic, chemical, or photolytic cleavage. Removal of a removable group, e.g., a blocking group, does not require that the entire removable group be removed, only that a sufficient portion of it be removed such that a DNA polymerase can extend a nucleic acid by incorporation of at least one additional nucleotide using a nucleotide or nucleotide analogue. In general, the conditions under which a removable group is removed are compatible with a process employing the removable group (e.g., an amplification process or sequencing process).

As used herein, the terms “reversible blocking groups” and “reversible terminators” are used in accordance with their plain and ordinary meanings and refer to a blocking moiety located, for example, at the 3′ position of a modified nucleotide and may be a chemically cleavable moiety such as an allyl group, an azidomethyl group or a methoxymethyl group, or may be an enzymatically cleavable group such as a phosphate ester. Non-limiting examples of nucleotide blocking moieties are described in applications WO 2004/018497, WO 96/07669, U.S. Pat. Nos. 7,057,026, 7,541,444, 5,763,594, 5,808,045, 5,872,244 and 6,232,465 the contents of which are incorporated herein by reference in their entirety. The nucleotides may be labelled or unlabeled. They may be modified with reversible terminators useful in methods provided herein and may be 3′-O-blocked reversible or 3′-unblocked reversible terminators. In nucleotides with 3′-O-blocked reversible terminators, the blocking group —OR [reversible terminating (capping) group] is linked to the oxygen atom of the 3′-OH of the pentose, while the label is linked to the base, which acts as a reporter and can be cleaved. The 3′-O-blocked reversible terminators are known in the art, and may be, for instance, a 3′-ONH₂ reversible terminator, a 3′-O-allyl reversible terminator, or a 3′-O-azidomethyl reversible terminator. In embodiments, the reversible terminator moiety is attached to the 3′-oxygen of the nucleotide, having the formula:

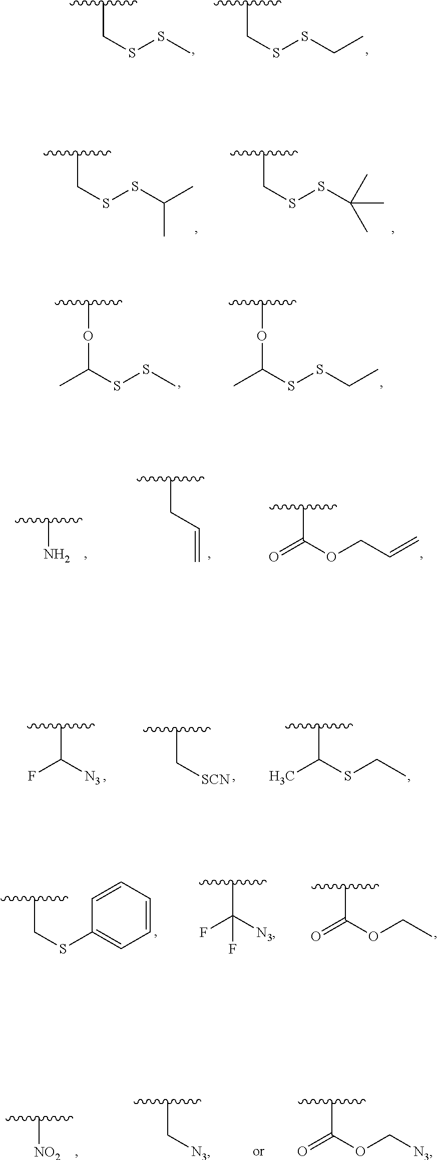

wherein the 3′ oxygen of the nucleotide is not shown in the formulae above. The term “allyl” as described herein refers to an unsubstituted methylene attached to a vinyl group (i.e., —CH═CH₂). In embodiments, the reversible terminator moiety is

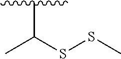 [SMILES: *C(C)SSC]

as described in U.S. Pat. No. 10,738,072, which is incorporated herein by reference for all purposes. For example, a nucleotide including a reversible terminator moiety may be represented by the formula:

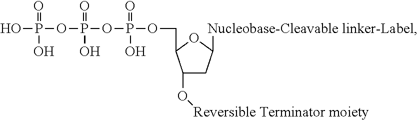

where the nucleobase is adenine or adenine analogue, thymine or thymine analogue, guanine or guanine analogue, or cytosine or cytosine analogue.

In some embodiments, a nucleic acid (e.g., a probe or a primer) includes a molecular identifier or a molecular barcode. As used herein, the term “molecular barcode” (which may be referred to as a “tag”, a “barcode”, a “molecular identifier”, an “identifier sequence” or a “unique molecular identifier” (UMI)) refers to any material (e.g., a nucleotide sequence, a nucleic acid molecule feature) that is capable of distinguishing an individual molecule in a large heterogeneous population of molecules. In embodiments, a barcode is unique in a pool of barcodes that differ from one another in sequence, or is uniquely associated with a particular sample polynucleotide in a pool of sample polynucleotides. In embodiments, every barcode in a pool of adapters is unique, such that sequencing reads including the barcode can be identified as originating from a single sample polynucleotide molecule on the basis of the barcode alone. In other embodiments, individual barcode sequences may be used more than once, but adapters including the duplicate barcodes are associated with different sequences and/or in different combinations of barcoded adaptors, such that sequence reads may still be uniquely distinguished as originating from a single sample polynucleotide molecule on the basis of a barcode and adjacent sequence information (e.g., sample polynucleotide sequence, and/or one or more adjacent barcodes). In embodiments, barcodes are about or at least about 5, 6, 7, 8, 9, 10, 15, 20, 25, 30, 40, 50, 75 or more nucleotides in length. In embodiments, barcodes are shorter than 20, 15, 10, 9, 8, 7, 6, or 5 nucleotides in length. In embodiments, barcodes are about 10 to about 50 nucleotides in length, such as about 15 to about 40 or about 20 to about 30 nucleotides in length. In a pool of different barcodes, barcodes may have the same or different lengths. In general, barcodes are of sufficient length and include sequences that are sufficiently different to allow the identification of sequencing reads that originate from the same sample polynucleotide molecule. In embodiments, each barcode in a plurality of barcodes differs from every other barcode in the plurality by at least three nucleotide positions, such as at least 3, 4, 5, 6, 7, 8, 9, 10, or more nucleotide positions. In some embodiments, substantially degenerate barcodes may be known as random. In some embodiments, a barcode may include a nucleic acid sequence from within a pool of known sequences. In some embodiments, the barcodes may be pre-defined. In embodiments, the barcodes are selected to form a known set of barcodes, e.g., the set of barcodes may be distinguished by a particular Hamming distance. In embodiments, each barcode sequence is unique within the known set of barcodes. In embodiments, each barcode sequence is associated with a particular oligonucleotide probe.

In embodiments, a nucleic acid (e.g., an adapter or primer) includes a sample barcode. In general, a “sample barcode” is a nucleotide sequence that is sufficiently different from other sample barcode to allow the identification of the sample source based on sample barcode sequence(s) with which they are associated. In embodiments, a plurality of nucleotides (e.g., all nucleotides from a particular sample source, or sub-sample thereof) are joined to a first sample barcode, while a different plurality of nucleotides (e.g., all nucleotides from a different sample source, or different subsample) are joined to a second sample barcode, thereby associating each plurality of polynucleotides with a different sample barcode indicative of sample source. In embodiments, each sample barcode in a plurality of sample barcodes differs from every other sample barcode in the plurality by at least three nucleotide positions, such as at least 3, 4, 5, 6, 7, 8, 9, 10, or more nucleotide positions. In some embodiments, substantially degenerate sample barcodes may be known as random. In some embodiments, a sample barcode may include a nucleic acid sequence from within a pool of known sequences, In some embodiments, the sample barcodes may be pre-defined. In embodiments, the sample barcode includes about 1 to about 10 nucleotides. In embodiments, the sample barcode includes about 3, 4, 5, 6, 7, 8, 9, or about 10 nucleotides. In embodiments, the sample barcode includes about 3 nucleotides. In embodiments, the sample barcode includes about 5 nucleotides. In embodiments, the sample barcode includes about 7 nucleotides. In embodiments, the sample barcode includes about 10 nucleotides. In embodiments, the sample barcode includes about 6 to about 10 nucleotides.

As used herein, the term “DNA polymerase” and “nucleic acid polymerase” are used in accordance with their plain ordinary meanings and refer to enzymes capable of synthesizing nucleic acid molecules from nucleotides (e.g., deoxyribonucleotides). Exemplary types of polymerases that may be used in the compositions and methods of the present disclosure include the nucleic acid polymerases such as DNA polymerase, DNA- or RNA-dependent RNA polymerase, and reverse transcriptase. In some cases, the DNA polymerase is 9° N polymerase or a variant thereof, E. coli DNA polymerase I, Bacteriophage T4 DNA polymerase, Sequenase, Taq DNA polymerase, DNA polymerase from Bacillus stearothermophilus, Bst 2.0 DNA polymerase, 9° N polymerase (exo-)A485L/Y409V, Phi29 DNA Polymerase (φ29 DNA Polymerase), T7 DNA polymerase, DNA polymerase II, DNA polymerase III holoenzyme, DNA polymerase IV, DNA polymerase V, VentR DNA polymerase, Therminator™ II DNA Polymerase, Therminator™ III DNA Polymerase, or Therminator™ IX DNA Polymerase. In embodiments, the polymerase is a protein polymerase. Typically, a DNA polymerase adds nucleotides to the 3′-end of a DNA strand, one nucleotide at a time. In embodiments, the DNA polymerase is a Pol I DNA polymerase, Pol II DNA polymerase, Pol III DNA polymerase, Pol IV DNA polymerase, Pol V DNA polymerase, Pol β DNA polymerase, Pol μ DNA polymerase, Pol λ DNA polymerase, Pol σ DNA polymerase, Pol α DNA polymerase, Pol δ DNA polymerase, Pol ε DNA polymerase, Pol η DNA polymerase, Pol ξ DNA polymerase, Pol κ DNA polymerase, Pol ζ DNA polymerase, Pol γ DNA polymerase, Pol θ DNA polymerase, Pol ν DNA polymerase, or a thermophilic nucleic acid polymerase (e.g. Therminator 7, 9° N polymerase (exo-), Therminator II, Therminator III, or Therminator IX). In embodiments, the DNA polymerase is a modified archaeal DNA polymerase. In embodiments, the polymerase is a reverse transcriptase. In embodiments, the polymerase is a mutant P. abyssi polymerase (e.g., such as a mutant P. abyssi polymerase described in WO 2018/148723 or WO 2020/056044). In embodiments, the polymerase is an enzyme described in US 2021/0139884. For example, a polymerase catalyzes the addition of a next correct nucleotide to the 3′-OH group of the primer via a phosphodiester bond, thereby chemically incorporating the nucleotide into the primer. Optionally, the polymerase used in the provided methods is a processive polymerase. Optionally, the polymerase used in the provided methods is a distributive polymerase.

As used herein, the term “thermophilic nucleic acid polymerase” refers to a family of DNA polymerases (e.g., 9° N™) and mutants thereof derived from the DNA polymerase originally isolated from the hyperthermophilic archaea, Thermococcus sp. 9 degrees N-7, found in hydrothermal vents at that latitude (East Pacific Rise) (Southworth M W, et al. PNAS. 1996; 93(11):5281-5285). A thermophilic nucleic acid polymerase is a member of the family B DNA polymerases. Site-directed mutagenesis of the 3′-5′ exo motif I (Asp-Ile-Glu or DIE) to AIA, AIE, EIE, EID or DIA yielded polymerase with no detectable 3′ exonuclease activity. Mutation to Asp-Ile-Asp (DID) resulted in reduction of 3′-5′ exonuclease specific activity to <1% of wild type, while maintaining other properties of the polymerase including its high strand displacement activity. The sequence AIA (D141A, E143A) was chosen for reducing exonuclease. Subsequent mutagenesis of key amino acids results in an increased ability of the enzyme to incorporate dideoxynucleotides, ribonucleotides and acyclonucleotides (e.g., Therminator II enzyme from New England Biolabs with D141A/E143A/Y409V/A485L mutations); 3′-amino-dNTPs, 3′-azido-dNTPs and other 3′-modified nucleotides (e.g., NEB Therminator III DNA Polymerase with D141A/E143A/L408S/Y409A/P410V mutations, NEB Therminator IX DNA polymerase), or 7-phosphate labeled nucleotides (e.g., Therminator 7: D141A/E143A/W355A/L408 W/R460A/Q461S/K464E/D480V/R484 W/A485L). Typically, these enzymes do not have 5′-3′ exonuclease activity. Additional information about thermophilic nucleic acid polymerases may be found in (Southworth M W, et al. PNAS. 1996; 93(11):5281-5285; Bergen K, et al. ChemBioChem. 2013; 14(9):1058-1062; Kumar S, et al. Scientific Reports. 2012; 2:684; Fuller C W, et al. 2016; 113(19):5233-5238; Guo J, et al. Proceedings of the National Academy of Sciences of the United States of America. 2008; 105(27):9145-9150), which are incorporated herein in their entirety for all purposes.

As used herein, the term “strand-displacing polymerase” refers to a type of polymerase (e.g., a DNA polymerase or reverse transcriptase) that is able to synthesize new DNA strands while simultaneously displacing the template strand in a single reaction. Strand-displacing polymerases are able to displace one or more nucleotides, for example 10 or 100 or more nucleotides, that are downstream from the enzyme. Strand-displacing polymerases are commonly used in isothermal amplification techniques, such as loop-mediated isothermal amplification (LAMP) and multiple displacement amplification (MDA). One example of a strand-displacing polymerase is the Bst DNA polymerase, which is commonly used in LAMP reactions. Another example is the phi29 DNA polymerase, which is often used in RCA reactions.

As used herein, the term “exonuclease activity” is used in accordance with its ordinary meaning in the art, and refers to the removal of a nucleotide from a nucleic acid by an enzyme (e.g. DNA polymerase, a lambda exonuclease, Exo I, Exo III, T5, Exo V, Exo VII or the like). For example, during polymerization, nucleotides are added to the 3′ end of the primer strand. Occasionally a DNA polymerase incorporates an incorrect nucleotide to the 3′-OH terminus of the primer strand, wherein the incorrect nucleotide cannot form a hydrogen bond to the corresponding base in the template strand. Such a nucleotide, added in error, is removed from the primer as a result of the 3′ to 5′ exonuclease activity of the DNA polymerase. In embodiments, exonuclease activity may be referred to as “proofreading.” When referring to 3′-5′ exonuclease activity, it is understood that the DNA polymerase facilitates a hydrolyzing reaction that breaks phosphodiester bonds at the 3′ end of a polynucleotide chain to excise the nucleotide. In embodiments, 3′-5′ exonuclease activity refers to the successive removal of nucleotides in single-stranded DNA in a 3′→5′ direction, releasing deoxyribonucleoside 5′-monophosphates one after another. Methods for quantifying exonuclease activity are known in the art, see for example Southworth et al, PNAS Vol 93, 8281-8285 (1996). In embodiments, 5′-3′ exonuclease activity refers to the successive removal of nucleotides in double-stranded DNA in a 5′→3′ direction. In embodiments, the 5′-3′ exonuclease is lambda exonuclease. For example, lambda exonuclease catalyzes the removal of 5′ mononucleotides from duplex DNA, with a preference for 5′ phosphorylated double-stranded DNA. In other embodiments, the 5′-3′ exonuclease is E. coli DNA Polymerase I.

As used herein, the term “incorporating” or “chemically incorporating,” when used in reference to a primer and cognate nucleotide, refers to the process of joining the cognate nucleotide to the primer or extension product thereof by formation of a phosphodiester bond.

As used herein, the term “selective” or “selectivity” or the like of a compound refers to the compound's ability to discriminate between molecular targets. For example, a chemical reagent may selectively modify one nucleotide type in that it reacts with one nucleotide type (e.g., cytosines) and not other nucleotide types (e.g., adenine, thymine, or guanine). When used in the context of sequencing, such as in “selectively sequencing,” this term refers to sequencing one or more target polynucleotides from an original starting population of polynucleotides, and not sequencing non-target polynucleotides from the starting population. Typically, selectively sequencing one or more target polynucleotides involves differentially manipulating the target polynucleotides based on known sequence. For example, target polynucleotides may be hybridized to a probe oligonucleotide that may be labeled (such as with a member of a binding pair) or bound to a surface. In embodiments, hybridizing a target polynucleotide to a probe oligonucleotide includes the step of displacing one strand of a double-stranded nucleic acid. Probe-hybridized target polynucleotides may then be separated from non-hybridized polynucleotides, such as by removing probe-bound polynucleotides from the starting population or by washing away polynucleotides that are not bound to a probe. The result is a selected subset of the starting population of polynucleotides, which is then subjected to sequencing, thereby selectively sequencing the one or more target polynucleotides.

As used herein, the term “template polynucleotide” refers to any polynucleotide molecule that may be bound by a polymerase and utilized as a template for nucleic acid synthesis. A template polynucleotide may be a target polynucleotide. In general, the term “target polynucleotide” refers to a nucleic acid molecule or polynucleotide in a starting population of nucleic acid molecules having a target sequence whose presence, amount, and/or nucleotide sequence, or changes in one or more of these, are desired to be determined. The target sequence may be a portion of a gene, a regulatory sequence, genomic DNA, cDNA, RNA including mRNA, miRNA, rRNA, or others. The target sequence may be a target sequence from a sample or a secondary target such as a product of an amplification reaction. A target polynucleotide is not necessarily any single molecule or sequence. For example, a target polynucleotide may be any one of a plurality of target polynucleotides in a reaction, or all polynucleotides in a given reaction, depending on the reaction conditions. For example, in a nucleic acid amplification reaction with random primers, all polynucleotides in a reaction may be amplified. As a further example, a collection of targets may be simultaneously assayed using polynucleotide primers directed to a plurality of targets in a single reaction. As yet another example, all or a subset of polynucleotides in a sample may be modified by the addition of a primer-binding sequence (such as by the ligation of adapters containing the primer binding sequence), rendering each modified polynucleotide a target polynucleotide in a reaction with the corresponding primer polynucleotide(s). In embodiments, the template polynucleotide includes a target nucleic acid sequence and one or more barcode sequences. In embodiments, the template polynucleotide is a barcode sequence. A “target sequence”, as used herein, refers to a sequence of a splint oligonucleotide that is the same, or substantially the same, as a sequence in a target polynucleotide (i.e., the target sequence of the splint oligonucleotide is the same, or substantially the same, as the target sequence in the target polynucleotide). In embodiments, the target sequence is a known sequence. In embodiments, the target sequence is selected from a set of known target sequences. In embodiments, the target sequence is located 5′ of the probe hybridization sequence of the target polynucleotide. A “subject sequence”, as used herein, refers to the sequence of interest in a target polynucleotide. For example, an oligonucleotide probe may be hybridized upstream of a subject sequence of a target polynucleotide and extending the oligonucleotide probe incorporates a sequence complementary to the subject sequence (i.e., a subject sequence complement) into the oligonucleotide probe. The extended oligonucleotide probe may then be processed further (e.g., circularized and/or amplified), and the subject sequence detected by, e.g., sequencing.

In embodiments, a target polynucleotide is a cell-free polynucleotide. In general, the terms “cell-free,” “circulating,” and “extracellular” as applied to polynucleotides (e.g. “cell-free DNA” (cfDNA) and “cell-free RNA” (cfRNA)) are used interchangeably to refer to polynucleotides present in a sample from a subject or portion thereof that can be isolated or otherwise manipulated without applying a lysis step to the sample as originally collected (e.g., as in extraction from cells or viruses). Cell-free polynucleotides are thus unencapsulated or “free” from the cells or viruses from which they originate, even before a sample of the subject is collected. Cell-free polynucleotides may be produced as a byproduct of cell death (e.g., apoptosis or necrosis) or cell shedding, releasing polynucleotides into surrounding body fluids or into circulation. Accordingly, cell-free polynucleotides may be isolated from a non-cellular fraction of blood (e.g., serum or plasma), from other bodily fluids (e.g., urine), or from non-cellular fractions of other types of samples.

As used herein, the terms “specific”, “specifically”, “specificity”, or the like of a compound refers to the compound's ability to cause a particular action, such as binding, to a particular molecular target with minimal or no action to other proteins in the cell.

The terms “attached,” “bind,” and “bound” as used herein are used in accordance with their plain and ordinary meanings and refer to an association between atoms or molecules. The association can be direct or indirect. For example, attached molecules may be directly bound to one another, e.g., by a covalent bond or non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like). As a further example, two molecules may be bound indirectly to one another by way of direct binding to one or more intermediate molecules, thereby forming a complex.

“Specific binding” is where the binding is selective between two molecules. A particular example of specific binding is that which occurs between an antibody and an antigen. Typically, specific binding can be distinguished from non-specific when the dissociation constant (KD) is less than about 1×10⁻⁵ M or less than about 1×10⁻⁶ M or 1×10⁻⁷ M. Specific binding can be detected, for example, by ELISA, immunoprecipitation, coprecipitation, with or without chemical crosslinking, two-hybrid assays and the like. In embodiments, the KD (equilibrium dissociation constant) between two specific binding molecules is less than 10-6 M, less than 10-7 M, less than 10-8 M, less than 10-9 M, less than 10-9 M, less than 10-11 M, or less than about 10-12 M or less.

As used herein, the terms “sequencing”, “sequence determination”, “determining a nucleotide sequence”, and the like include determination of a partial or complete sequence information (e.g., a sequence) of a polynucleotide being sequenced, and particularly physical processes for generating such sequence information. That is, the term includes sequence comparisons, consensus sequence determination, contig assembly, fingerprinting, and like levels of information about a target polynucleotide, as well as the express identification and ordering of nucleotides in a target polynucleotide. The term also includes the determination of the identification, ordering, and locations of one, two, or three of the four types of nucleotides within a target polynucleotide. In some embodiments, a sequencing process described herein includes contacting a template and an annealed primer with a suitable polymerase under conditions suitable for polymerase extension and/or sequencing.

As used herein, the term “polymer” refers to macromolecules having one or more structurally unique repeating units. The repeating units are referred to as “monomers,” which are polymerized for the polymer. Typically, a polymer is formed by monomers linked in a chain-like structure. A polymer formed entirely from a single type of monomer is referred to as a “homopolymer.” A polymer formed from two or more unique repeating structural units may be referred to as a “copolymer.” A polymer may be linear or branched, and may be random, block, polymer brush, hyperbranched polymer, bottlebrush polymer, dendritic polymer, or polymer micelles. The term “polymer” includes homopolymers, copolymers, tripolymers, tetra polymers and other polymeric molecules made from monomeric subunits. Copolymers include alternating copolymers, periodic copolymers, statistical copolymers, random copolymers, block copolymers, linear copolymers and branched copolymers. The term “polymerizable monomer” is used in accordance with its meaning in the art of polymer chemistry and refers to a compound that may covalently bind chemically to other monomer molecules (such as other polymerizable monomers that are the same or different) to form a polymer.

Polymers can be hydrophilic, hydrophobic or amphiphilic, as known in the art. Thus, “hydrophilic polymers” are substantially miscible with water and include, but are not limited to, polyethylene glycol and the like. “Hydrophobic polymers” are substantially immiscible with water and include, but are not limited to, polyethylene, polypropylene, polybutadiene, polystyrene, polymers disclosed herein, and the like. “Amphiphilic polymers” have both hydrophilic and hydrophobic properties and are typically copolymers having hydrophilic segment(s) and hydrophobic segment(s). Polymers include homopolymers, random copolymers, and block copolymers, as known in the art. The term “homopolymer” refers, in the usual and customary sense, to a polymer having a single monomeric unit. The term “copolymer” refers to a polymer derived from two or more monomeric species. The term “random copolymer” refers to a polymer derived from two or more monomeric species with no preferred ordering of the monomeric species. The term “block copolymer” refers to polymers having two or homopolymer subunits linked by covalent bond. Thus, the term “hydrophobic homopolymer” refers to a homopolymer which is hydrophobic. The term “hydrophobic block copolymer” refers to two or more homopolymer subunits linked by covalent bonds and which is hydrophobic.

As used herein, the term “hydrogel” refers to a three-dimensional polymeric structure that is substantially insoluble in water, but which is capable of absorbing and retaining large quantities of water to form a substantially stable, often soft and pliable, structure. In embodiments, water can penetrate in between polymer chains of a polymer network, subsequently causing swelling and the formation of a hydrogel. In embodiments, hydrogels are super-absorbent (e.g., containing more than about 90% water) and can be comprised of natural or synthetic polymers.

As used herein, the term “substrate” refers to a solid support material. The substrate can be non-porous or porous. The substrate can be rigid or flexible. As used herein, the terms “solid support” and “solid surface” refers to discrete solid or semi-solid surface. A solid support may encompass any type of solid, porous, or hollow sphere, ball, cylinder, or other similar configuration composed of plastic, ceramic, metal, or polymeric material (e.g., hydrogel) onto which a nucleic acid may be immobilized (e.g., covalently or non-covalently). A nonporous substrate generally provides a seal against bulk flow of liquids or gases. Exemplary solid supports include, but are not limited to, glass and modified or functionalized glass, plastics (including acrylics, polystyrene and copolymers of styrene and other materials, polypropylene, polyethylene, polybutylene, polyurethanes, Teflon™, cyclic olefin copolymers, polyimides etc.), nylon, ceramics, resins, Zeonor, silica or silica-based materials including silicon and modified silicon, carbon, metals, inorganic glasses, optical fiber bundles, photopatternable dry film resists, UV-cured adhesives and polymers. Particularly useful solid supports for some embodiments have at least one surface located within a flow cell. Solid surfaces can also be varied in their shape depending on the application in a method described herein. For example, a solid surface useful herein can be planar, or contain regions which are concave or convex. In embodiments, the geometry of the concave or convex regions (e.g., wells) of the solid surface conform to the size and shape of the particle to maximize the contact between as substantially circular particle. In embodiments, the wells of an array are randomly located such that nearest neighbor features have random spacing between each other. Alternatively, in embodiments the spacing between the wells can be ordered, for example, forming a regular pattern. The term solid substrate is encompassing of a substrate (e.g., a flow cell) having a surface including a polymer coating covalently attached thereto. In embodiments, the solid substrate is a flow cell. The term “flow cell” as used herein refers to a chamber including a solid surface across which one or more fluid reagents can be flowed. Examples of flow cells and related fluidic systems and detection platforms that can be readily used in the methods of the present disclosure are described, for example, in Bentley et al., Nature 456:53-59 (2008). In embodiments a substrate includes a surface (e.g., a surface of a flow cell, a surface of a tube, a surface of a chip), for example a metal surface (e.g., steel, gold, silver, aluminum, silicon and copper). In embodiments a substrate (e.g., a substrate surface) is coated and/or includes functional groups and/or inert materials. In embodiments a substrate includes a bead, a chip, a capillary, a plate, a membrane, a wafer (e.g., silicon wafers), a comb, or a pin for example. In some embodiments a substrate includes a bead and/or a nanoparticle. A substrate can be made of a suitable material, non-limiting examples of which include a plastic or a suitable polymer (e.g., polycarbonate, poly(vinyl alcohol), poly(divinylbenzene), polystyrene, polyamide, polyester, polyvinylidene difluoride (PVDF), polyethylene, polyurethane, polypropylene, and the like), borosilicate, glass, nylon, Wang resin, Merrifield resin, metal (e.g., iron, a metal alloy, sepharose, agarose, polyacrylamide, dextran, cellulose and the like or combinations thereof. In embodiments a substrate includes a magnetic material (e.g., iron, nickel, cobalt, platinum, aluminum, and the like). In embodiments a substrate includes a magnetic bead (e.g., DYNABEADS®, hematite, AMPure XP). Magnets can be used to purify and/or capture nucleic acids bound to substrates (e.g., substrates including a metal or magnetic material). The flow cell is typically a glass slide containing small fluidic channels (e.g., a glass slide 75 mm×25 mm×1 mm having one or more channels), through which sequencing solutions (e.g., polymerases, nucleotides, and buffers) may traverse. Though typically glass, suitable flow cell materials may include polymeric materials, plastics, silicon, quartz (fused silica), Borofloat® glass, silica, silica-based materials, carbon, metals, an optical fiber or optical fiber bundles, sapphire, or plastic materials such as COCs and epoxies. The particular material can be selected based on properties desired for a particular use. For example, materials that are transparent to a desired wavelength of radiation are useful for analytical techniques that will utilize radiation of the desired wavelength. Conversely, it may be desirable to select a material that does not pass radiation of a certain wavelength (e.g., being opaque, absorptive, or reflective). In embodiments, the material of the flow cell is selected due to the ability to conduct thermal energy. In embodiments, a flow cell includes inlet and outlet ports and a flow channel extending there between.

The term “surface” is intended to mean an external part or external layer of a substrate. The surface can be in contact with another material such as a gas, liquid, gel, polymer, organic polymer, second surface of a similar or different material, metal, or coat. The surface, or regions thereof, can be substantially flat. The substrate and/or the surface can have surface features such as wells, pits, channels, ridges, raised regions, pegs, posts or the like.

The term “microplate”, or “multiwell container” as used herein, refers to a substrate including a surface, the surface including a plurality of reaction chambers separated from each other by interstitial regions on the surface. In embodiments, the microplate has dimensions as provided and described by American National Standards Institute (ANSI) and Society for Laboratory Automation And Screening (SLAS); for example the tolerances and dimensions set forth in ANSI SLAS 1-2004 (R2012); ANSI SLAS 2-2004 (R2012); ANSI SLAS 3-2004 (R2012); ANSI SLAS 4-2004 (R2012); and ANSI SLAS 6-2012, which are incorporated herein by reference. The dimensions of the microplate as described herein and the arrangement of the reaction chambers may be compatible with an established format for automated laboratory equipment. In embodiments, the device described herein provides methods for high-throughput screening. High-throughput screening (HTS) refers to a process that uses a combination of modern robotics, data processing and control software, liquid handling devices, and/or sensitive detectors, to efficiently process a large amount of (e.g., thousands, hundreds of thousands, or millions) samples in biochemical, genetic, or pharmacological experiments, either in parallel or in sequence, within a reasonably short period of time (e.g., days). Preferably, the process is amenable to automation, such as robotic simultaneous handling of 96 samples, 384 samples, 1536 samples or more. A typical HTS robot tests up to 100,000 to a few hundred thousand compounds per day. The samples are often in small volumes, such as no more than 1 mL, 500 μl, 200 μl, 100 μl, 50 μl or less. Through this process, one can rapidly identify active compounds, small molecules, antibodies, proteins or polynucleotides in a cell.

The reaction chambers may be provided as wells of a multiwell container (alternatively referred to as reaction chambers), for example a microplate may contain 2, 4, 6, 12, 24, 48, 96, 384, or 1536 sample wells. In embodiments, the 96 and 384 wells are arranged in a 2:3 rectangular matrix. In embodiments, the 24 wells are arranged in a 3:8 rectangular matrix. In embodiments, the 48 wells are arranged in a 3:4 rectangular matrix. In embodiments, the reaction chamber is a microscope slide (e.g., a glass slide about 75 mm by about 25 mm). In embodiments the slide is a concavity slide (e.g., the slide includes a depression). In embodiments, the slide includes a coating for enhanced cell adhesion (e.g., poly-L-lysine, silanes, carbon nanotubes, polymers, epoxy resins, or gold). In embodiments, the microplate is about 5 inches by about 3.33 inches, and includes a plurality of 5 mm diameter wells. In embodiments, the microplate is about 5 inches by about 3.33 inches, and includes a plurality of 6 mm diameter wells. In embodiments, the microplate is about 5 inches by about 3.33 inches, and includes a plurality of 7 mm diameter wells. In embodiments, the microplate is about 5 inches by about 3.33 inches, and includes a plurality of 7.5 mm diameter wells. In embodiments, the microplate is 5 inches by 3.33 inches, and includes a plurality of 7.5 mm diameter wells. In embodiments, the microplate is about 5 inches by about 3.33 inches, and includes a plurality of 8 mm diameter wells. In embodiments, the microplate is a flat glass or plastic tray in which an array of wells are formed, wherein each well can hold between from a few microliters to hundreds of microliters of fluid reagents and samples. In embodiments, the microplate has a rectangular shape that measures 127.7 mm±0.5 mm in length by 85.4 mm±0.5 mm in width, and includes 6, 12, 24, 48, or 96 wells, wherein each well has an average diameter of about 5-7 mm. In embodiments, the microplate has a rectangular shape that measures 127.7 mm±0.5 mm in length by 85.4 mm±0.5 mm in width, and includes 6, 12, 24, 48, or 96 wells, wherein each well has an average diameter of about 6 mm.

The term “well” refers to a discrete concave feature in a substrate having a surface opening that is completely surrounded by interstitial region(s) of the surface. Wells can have any of a variety of shapes at their opening in a surface including but not limited to round, elliptical, square, polygonal, or star shaped (i.e., star shaped with any number of vertices). The cross section of a well taken orthogonally with the surface may be curved, square, polygonal, hyperbolic, conical, or angular. The wells of a microplate are available in different shapes, for example F-Bottom: flat bottom; C-Bottom: bottom with minimal rounded edges; V-Bottom: V-shaped bottom; or U-Bottom: U-shaped bottom. In embodiments, the well is substantially square. In embodiments, the well is square. In embodiments, the well is F-bottom. In embodiments, the microplate includes 24 substantially round flat bottom wells. In embodiments, the microplate includes 48 substantially round flat bottom wells. In embodiments, the microplate includes 96 substantially round flat bottom wells. In embodiments, the microplate includes 384 substantially square flat bottom wells.

The discrete regions (i.e., features, wells) of the microplate may have defined locations in a regular array, which may correspond to a rectilinear pattern, circular pattern, hexagonal pattern, or the like. In embodiments, the pattern of wells includes concentric circles of regions, spiral patterns, rectilinear patterns, hexagonal patterns, and the like. In embodiments, the pattern of wells is arranged in a rectilinear or hexagonal pattern A regular array of such regions is advantageous for detection and data analysis of signals collected from the arrays during an analysis. These discrete regions are separated by interstitial regions. As used herein, the term “interstitial region” refers to an area in a substrate or on a surface that separates other areas of the substrate or surface. For example, an interstitial region can separate one concave feature of an array from another concave feature of the array. The two regions that are separated from each other can be discrete, lacking contact with each other. In another example, an interstitial region can separate a first portion of a feature from a second portion of a feature. In embodiments the interstitial region is continuous whereas the features are discrete, for example, as is the case for an array of wells in an otherwise continuous surface. The separation provided by an interstitial region can be partial or full separation. In embodiments, interstitial regions have a surface material that differs from the surface material of the wells (e.g., the interstitial region contains a photoresist and the surface of the well is glass). In embodiments, interstitial regions have a surface material that is the same as the surface material of the wells (e.g., both the surface of the interstitial region and the surface of well contain a polymer or copolymer).

As used herein, the term “sequencing reaction mixture” is used in accordance with its plain and ordinary meaning and refers to an aqueous mixture that contains the reagents necessary to allow dNTP or dNTP analogue (e.g., a modified nucleotide) to add a nucleotide to a DNA strand by a DNA polymerase. In embodiments, the sequencing reaction mixture includes a buffer. In embodiments, the buffer includes an acetate buffer, 3-(N-morpholino)propanesulfonic acid (MOPS) buffer, N-(2-Acetamido)-2-aminoethanesulfonic acid (ACES) buffer, phosphate-buffered saline (PBS) buffer, 4-(2-hydroxyethyl)-1-piperazineethanesulfonic acid (HEPES) buffer, N-(1,1-Dimethyl-2-hydroxyethyl)-3-amino-2-hydroxypropanesulfonic acid (AMPSO) buffer, borate buffer (e.g., borate buffered saline, sodium borate buffer, boric acid buffer), 2-Amino-2-methyl-1,3-propanediol (AMPD) buffer, N-cyclohexyl-2-hydroxyl-3-aminopropanesulfonic acid (CAPSO) buffer, 2-Amino-2-methyl-1-propanol (AMP) buffer, 4-(cyclohexylamino)-1-butanesulfonic acid (CABS) buffer, glycine-NaOH buffer, N-Cyclohexyl-2-aminoethanesulfonic acid (CHES) buffer, tris(hydroxymethyl)aminomethane (Tris) buffer, or a N-cyclohexyl-3-aminopropanesulfonic acid (CAPS) buffer. In embodiments, the buffer is a borate buffer. In embodiments, the buffer is a CHES buffer. In embodiments, the sequencing reaction mixture includes nucleotides, wherein the nucleotides include a reversible terminating moiety and a label covalently linked to the nucleotide via a cleavable linker. In embodiments, the sequencing reaction mixture includes a buffer, DNA polymerase, detergent (e.g., Triton X), a chelator (e.g., EDTA), and/or salts (e.g., ammonium sulfate, magnesium chloride, sodium chloride, or potassium chloride).

As used herein, the term “sequencing cycle” is used in accordance with its plain and ordinary meaning and refers to incorporating one or more nucleotides (e.g., nucleotide analogues) to the 3′ end of a polynucleotide with a polymerase, and detecting one or more labels that identify the one or more nucleotides incorporated. In embodiments, one nucleotide (e.g., a modified nucleotide) is incorporated per sequencing cycle. The sequencing may be accomplished by, for example, sequencing by synthesis, pyrosequencing, and the like. In embodiments, a sequencing cycle includes extending a complementary polynucleotide by incorporating a first nucleotide using a polymerase, wherein the polynucleotide is hybridized to a template nucleic acid, detecting the first nucleotide, and identifying the first nucleotide. In embodiments, to begin a sequencing cycle, one or more differently labeled nucleotides and a DNA polymerase can be introduced. Following nucleotide addition, signals produced (e.g., via excitation and emission of a detectable label) can be detected to determine the identity of the incorporated nucleotide (based on the labels on the nucleotides). Reagents can then be added to remove the 3′ reversible terminator and to remove labels from each incorporated base. Reagents, enzymes, and other substances can be removed between steps by washing. Cycles may include repeating these steps, and the sequence of each cluster is read over the multiple repetitions.

As used herein, the term “extension” or “elongation” is used in accordance with their plain and ordinary meanings and refer to synthesis by a polymerase of a new polynucleotide strand complementary to a template strand by adding free nucleotides (e.g., dNTPs) from a reaction mixture that are complementary to the template in the 5′-to-3′ direction. Extension includes condensing the 5′-phosphate group of the dNTPs with the 3′-hydroxy group at the end of the nascent (elongating) DNA strand.

As used herein, the term “sequencing read” is used in accordance with its plain and ordinary meaning and refers to an inferred sequence of nucleotide bases (or nucleotide base probabilities) corresponding to all or part of a single polynucleotide fragment. A sequencing read may include 10, 20, 30, 40, 50, 60, 70, 80, 90, 100, 150, 200, 250, or more nucleotide bases. In embodiments, a sequencing read includes reading a barcode sequence and a template nucleotide sequence. In embodiments, a sequencing read includes reading a template nucleotide sequence. In embodiments, a sequencing read includes reading a barcode and not a template nucleotide sequence. Reads of length 20-40 base pairs (bp) are referred to as ultra-short. Typical sequencers produce read lengths in the range of 100-500 bp. Read length is a factor which can affect the results of biological studies. For example, longer read lengths improve the resolution of de novo genome assembly and detection of structural variants. In embodiments, a sequencing read includes reading a barcode and a template nucleotide sequence. In embodiments, a sequencing read includes reading a template nucleotide sequence. In embodiments, a sequencing read includes reading a barcode and not a template nucleotide sequence. In embodiments, a sequencing read includes a computationally derived string corresponding to the detected label. In some embodiments, a sequencing read may include 300, 400, 500, 600, 700, 800, 900, 1,000, 1,100, 1,200, 1,300, 1,400, 1,500, or more nucleotide bases.

The term “multiplexing” as used herein refers to an analytical method in which the presence and/or amount of multiple targets, e.g., multiple nucleic acid target sequences, can be assayed simultaneously by using the methods and devices as described herein, each of which has at least one different detection characteristic, e.g., fluorescence characteristic (for example excitation wavelength, emission wavelength, emission intensity, FWHM (full width at half maximum peak height), or fluorescence lifetime) or a unique nucleic acid or protein sequence characteristic. As used herein, the term “multiplex” is used to refer to an assay in which multiple (i.e. at least two) different biomolecules are assayed at the same time, and more particularly in the same aliquot of the sample, or in the same reaction mixture. In embodiments, more than two different biomolecules are assayed at the same time. In embodiments, at least 2, 4, 6, 8, 10, 20, 50, 100, 200, 300, 400, 500, 600, 700, 800, 900, 1000, 1100, 1200, 1300, 1400 or 1500 or more biomolecules are detected according to the present method.

Complementary single stranded nucleic acids and/or substantially complementary single stranded nucleic acids can hybridize to each other under hybridization conditions, thereby forming a nucleic acid that is partially or fully double stranded. All or a portion of a nucleic acid sequence may be substantially complementary to another nucleic acid sequence, in some embodiments. As referred to herein, “substantially complementary” refers to nucleotide sequences that can hybridize with each other under suitable hybridization conditions. Hybridization conditions can be altered to tolerate varying amounts of sequence mismatch within complementary nucleic acids that are substantially complementary. Substantially complementary portions of nucleic acids that can hybridize to each other can be 75% or more, 76% or more, 77% or more, 78% or more, 79% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more or 99% or more complementary to each other. In some embodiments substantially complementary portions of nucleic acids that can hybridize to each other are 100% complementary. Nucleic acids, or portions thereof, that are configured to hybridize to each other often include nucleic acid sequences that are substantially complementary to each other.

“Hybridize” shall mean the annealing of a nucleic acid sequence to another nucleic acid sequence (e.g., one single-stranded nucleic acid (such as a primer) to another nucleic acid) based on the well-understood principle of sequence complementarity. In an embodiment the other nucleic acid is a single-stranded nucleic acid. In some embodiments, one portion of a nucleic acid hybridizes to itself, such as in the formation of a hairpin structure. The propensity for hybridization between nucleic acids depends on the temperature and ionic strength of their milieu, the length of the nucleic acids and the degree of complementarity. The effect of these parameters on hybridization is described in, for example, Sambrook J., Fritsch E. F., Maniatis T., Molecular cloning: a laboratory manual, Cold Spring Harbor Laboratory Press, New York (1989). As used herein, hybridization of a primer, or of a DNA extension product, respectively, is extendable by creation of a phosphodiester bond with an available nucleotide or nucleotide analogue capable of forming a phosphodiester bond, therewith. For example, hybridization can be performed at a temperature ranging from 15° C. to 95° C. In some embodiments, the hybridization is performed at a temperature of about 20° C., about 25° C., about 30° C., about 35° C., about 40° C., about 45° C., about 50° C., about 55° C., about 60° C., about 65° C., about 70° C., about 75° C., about 80° C., about 85° C., about 90° C., or about 95° C. In other embodiments, the stringency of the hybridization can be further altered by the addition or removal of components of the buffered solution.

As used herein, “specifically hybridizes” refers to preferential hybridization under hybridization conditions where two nucleic acids, or portions thereof, that are substantially complementary, hybridize to each other and not to other nucleic acids that are not substantially complementary to either of the two nucleic acids. For example, specific hybridization includes the hybridization of a primer or capture nucleic acid to a portion of a target nucleic acid (e.g., a template, or adapter portion of a template) that is substantially complementary to the primer or capture nucleic acid. In some embodiments nucleic acids, or portions thereof, that are configured to specifically hybridize are often about 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, 99% or more or 100% complementary to each other over a contiguous portion of nucleic acid sequence. A specific hybridization discriminates over non-specific hybridization interactions (e.g., two nucleic acids that a not configured to specifically hybridize, e.g., two nucleic acids that are 80% or less, 70% or less, 60% or less or 50% or less complementary) by about 2-fold or more, often about 10-fold or more, and sometimes about 100-fold or more, 1000-fold or more, 10,000-fold or more, 100,000-fold or more, or 1,000,000-fold or more. Two nucleic acid strands that are hybridized to each other can form a duplex which includes a double stranded portion of nucleic acid.

As used herein, the term “adjacent,” refers to two nucleotide sequences in a nucleic acid, can refer to nucleotide sequences separated by 0 to about 20 nucleotides, more specifically, in a range of about 1 to about 10 nucleotides, or to sequences that directly abut one another. As those of skill in the art appreciate, two nucleotide sequences that that are to ligated together will generally directly abut one another.

A nucleic acid can be amplified by a suitable method. The term “amplification,” “amplified” or “amplifying” as used herein refers to subjecting a target nucleic acid in a sample to a process that linearly or exponentially generates amplicon nucleic acids having the same or substantially the same (e.g., substantially identical) nucleotide sequence as the target nucleic acid, or segment thereof, and/or a complement thereof (which may be referred to herein as an “amplification product” or “amplification products”). In some embodiments an amplification reaction includes a suitable thermal stable polymerase. Thermal stable polymerases are known and are stable for prolonged periods of time, at temperature greater than 80° C. when compared to common polymerases found in most mammals. In embodiments the term “amplification,” “amplified” or “amplifying” refers to a method that includes a polymerase chain reaction (PCR). Conditions conducive to amplification (i.e., amplification conditions) are known and often include at least a suitable polymerase, a suitable template, a suitable primer or set of primers, suitable nucleotides (e.g., dNTPs), a suitable buffer, and application of suitable annealing, hybridization and/or extension times and temperatures. In embodiments an amplified product (e.g., an amplicon) can contain one or more additional and/or different nucleotides than the template sequence, or portion thereof, from which the amplicon was generated (e.g., a primer can contain “extra” nucleotides (such as a 5′ portion that does not hybridize to the template), or one or more mismatched bases within a hybridizing portion of the primer).

Amplification according to the present teachings encompasses any means by which at least a part of at least one target nucleic acid is reproduced, typically in a template-dependent manner, including without limitation, a broad range of techniques for amplifying nucleic acid sequences, either linearly or exponentially. Illustrative means for performing an amplifying step include ligase chain reaction (LCR), ligase detection reaction (LDR), ligation followed by Q-replicase amplification, PCR, primer extension, strand displacement amplification (SDA), hyperbranched strand displacement amplification, multiple displacement amplification (MDA), nucleic acid strand-based amplification (NASBA), two-step multiplexed amplifications, rolling circle amplification (RCA), and the like, including multiplex versions and combinations thereof, for example but not limited to, OLA (oligonucleotide ligation assay)/PCR, PCR/OLA, LDR/PCR, PCR/PCR/LDR, PCR/LDR, LCR/PCR, PCR/LCR (also known as combined chain reaction—CCR), and the like. Descriptions of such techniques can be found in, among other sources, Ausbel et al.; PCR Primer: A Laboratory Manual, Diffenbach, Ed., Cold Spring Harbor Press (1995); The Electronic Protocol Book, Chang Bioscience (2002); Msuih et al., J. Clin. Micro. 34:501-07 (1996); The Nucleic Acid Protocols Handbook, R. Rapley, ed., Humana Press, Totowa, N.J. (2002); Abramson et al., Curr Opin Biotechnol. 1993 February; 4(1):41-7, U.S. Pat. Nos. 6,027,998; 6,605,451, Barany et al., PCT Publication No. WO 97/31256; Wenz et al., PCT Publication No. WO 01/92579; Day et al., Genomics, 29(1): 152-162 (1995), Ehrlich et al., Science 252:1643-50 (1991); Innis et al., PCR Protocols: A Guide to Methods and Applications, Academic Press (1990); Favis et al., Nature Biotechnology 18:561-64 (2000); and Rabenau et al., Infection 28:97-102 (2000); Belgrader, Barany, and Lubin, Development of a Multiplex Ligation Detection Reaction DNA Typing Assay, Sixth International Symposium on Human Identification, 1995 (available on the world wide web at: promega.com/geneticidproc/ussymp6proc/blegrad.html); LCR Kit Instruction Manual, Cat. #200520, Rev. #050002, Stratagene, 2002; Barany, Proc. Natl. Acad. Sci. USA 88:188-93 (1991); Bi and Sambrook, Nucl. Acids Res. 25:2924-2951 (1997); Zirvi et al., Nucl. Acid Res. 27:e40i-viii (1999); Dean et al., Proc Natl Acad Sci USA 99:5261-66 (2002); Barany and Gelfand, Gene 109:1-11 (1991); Walker et al., Nucl. Acid Res. 20:1691-96 (1992); Polstra et al., BMC Inf. Dis. 2:18-(2002); Lage et al., Genome Res. 2003 February; 13(2):294-307, and Landegren et al., Science 241:1077-80 (1988), Demidov, V., Expert Rev Mol Diagn. 2002 November; 2(6):542-8, Cook et al., J Microbiol Methods. 2003 May; 53(2):165-74, Schweitzer et al., Curr Opin Biotechnol. 2001 February; 12(1):21-7, U.S. Pat. Nos. 5,830,711, 6,027,889, 5,686,243, PCT Publication No. WO0056927A3, and PCT Publication No. WO9803673A1.

In some embodiments, amplification includes at least one cycle of the sequential procedures of: annealing at least one primer with complementary or substantially complementary sequences in at least one target nucleic acid; synthesizing at least one strand of nucleotides in a template-dependent manner using a polymerase; and denaturing the newly-formed nucleic acid duplex to separate the strands. The cycle may or may not be repeated. Amplification can include thermocycling or can be performed isothermally.

As used herein, the term “rolling circle amplification (RCA)” refers to a nucleic acid amplification reaction that amplifies a circular nucleic acid template (e.g., single-stranded DNA circles) via a rolling circle mechanism. Rolling circle amplification reaction is initiated by the hybridization of a primer to a circular, often single-stranded, nucleic acid template. The nucleic acid polymerase then extends the primer that is hybridized to the circular nucleic acid template by continuously progressing around the circular nucleic acid template to replicate the sequence of the nucleic acid template over and over again (rolling circle mechanism). The rolling circle amplification typically produces concatemers including tandem repeat units of the circular nucleic acid template sequence. The rolling circle amplification may be a linear RCA (LRCA), exhibiting linear amplification kinetics (e.g., RCA using a single specific primer), or may be an exponential RCA (ERCA) exhibiting exponential amplification kinetics. Rolling circle amplification may also be performed using multiple primers (multiply primed rolling circle amplification or MPRCA) leading to hyper-branched concatemers. For example, in a double-primed RCA, one primer may be complementary, as in the linear RCA, to the circular nucleic acid template, whereas the other may be complementary to the tandem repeat unit nucleic acid sequences of the RCA product. Consequently, the double-primed RCA may proceed as a chain reaction with exponential (geometric) amplification kinetics featuring a ramifying cascade of multiple-hybridization, primer-extension, and strand-displacement events involving both the primers. This often generates a discrete set of concatemeric, double-stranded nucleic acid amplification products. The rolling circle amplification may be performed in-vitro under isothermal conditions using a suitable nucleic acid polymerase such as Phi29 DNA polymerase. RCA may be performed by using any of the DNA polymerases that are known in the art (e.g., a Phi29 DNA polymerase, a Bst DNA polymerase, or SD polymerase).

As used herein, the term “circularizing” refers to the conversion of a linear nucleic acid molecule into a circular form. Circularization of a linear nucleic acid molecule, such as DNA or RNA, involves covalently linking the two ends of the molecule together to form a closed circle. Circularization may be obtained by, for example, association of complementary single stranded ends (sticky ends). Circularization may also be obtained by ligating the two ends of the linear nucleic acids. The ligation can be blunt-end ligation or sticky-end ligation. Circularizing may also be facilitated by the use of a splint oligonucleotide. For example, the two ends of a linear nucleic acid molecule are hybridized to two regions of a splint oligonucleotide such that the ends (i.e., the 5′ and 3′ ends) of the linear nucleic acid molecule are adjacent to each other, and a ligase is then used, for example, to covalently link the two ends together.

A nucleic acid can be amplified by a thermocycling method or by an isothermal amplification method. In some embodiments a rolling circle amplification method is used. In some embodiments amplification takes place on a solid support (e.g., within a flow cell) where a nucleic acid, nucleic acid library or portion thereof is immobilized. In certain sequencing methods, a nucleic acid library is added to a flow cell and immobilized by hybridization to anchors under suitable conditions. This type of nucleic acid amplification is often referred to as solid phase amplification. In some embodiments of solid phase amplification, all or a portion of the amplified products are synthesized by an extension initiating from an immobilized primer. Solid phase amplification reactions are analogous to standard solution phase amplifications except that at least one of the amplification oligonucleotides (e.g., primers) is immobilized on a solid support.

As used herein, the terms “cluster” and “colony” are used interchangeably to refer to a discrete site on a solid support that includes a plurality of immobilized polynucleotides and a plurality of immobilized complementary polynucleotides. The term “clustered array” refers to an array formed from such clusters or colonies. In this context the term “array” is not to be understood as requiring an ordered arrangement of clusters. The term “array” is used in accordance with its ordinary meaning in the art, and refers to a population of different molecules that are attached to one or more solid-phase substrates such that the different molecules can be differentiated from each other according to their relative location. An array can include different molecules that are each located at different addressable features on a solid-phase substrate. The molecules of the array can be nucleic acid primers, nucleic acid probes, nucleic acid templates or nucleic acid enzymes such as polymerases or ligases. Arrays useful in the invention can have densities that ranges from about 2 different features to many millions, billions or higher. The density of an array can be from 2 to as many as a billion or more different features per square cm. For example an array can have at least about 100 features/cm², at least about 1,000 features/cm², at least about 10,000 features/cm², at least about 100,000 features/cm², at least about 10,000,000 features/cm², at least about 100,000,000 features/cm², at least about 1,000,000,000 features/cm², at least about 2,000,000,000 features/cm² or higher. In embodiments, the arrays have features at any of a variety of densities including, for example, at least about 10 features/cm², 100 features/cm², 500 features/cm², 1,000 features/cm², 5,000 features/cm², 10,000 features/cm², 50,000 features/cm², 100,000 features/cm², 1,000,000 features/cm², 5,000,000 features/cm², or higher.

Provided herein are methods, systems, and compositions for analyzing a sample (e.g., sequencing nucleic acids within a sample) in situ. The term “in situ” is used in accordance with its ordinary meaning in the art and refers to a sample surrounded by at least a portion of its native environment, such as may preserve the relative position of two or more elements. For example, an extracted human cell obtained is considered in situ when the cell is retained in its local microenvironment so as to avoid extracting the target (e.g., nucleic acid molecules or proteins) away from their native environment. An in situ sample (e.g., a cell) can be obtained from a suitable subject. An in situ cell sample may refer to a cell and its surrounding milieu, or a tissue. A sample can be isolated or obtained directly from a subject or part thereof. In embodiments, the methods described herein (e.g., sequencing a plurality of target nucleic acids of a cell in situ) are applied to an isolated cell (i.e., a cell not surrounded by least a portion of its native environment). For the avoidance of any doubt, when the method is performed within a cell (e.g., an isolated cell) the method may be considered in situ. In some embodiments, a sample is obtained indirectly from an individual or medical professional. A sample can be any specimen that is isolated or obtained from a subject or part thereof. A sample can be any specimen that is isolated or obtained from multiple subjects. Non-limiting examples of tissues include organ tissues (e.g., liver, kidney, lung, thymus, adrenals, skin, bladder, reproductive organs, intestine, colon, spleen, brain, the like or parts thereof), epithelial tissue, hair, hair follicles, ducts, canals, bone, eye, nose, mouth, throat, ear, nails, the like, parts thereof or combinations thereof. A sample may include cells or tissues that are normal, healthy, diseased (e.g., infected), and/or cancerous (e.g., cancer cells). A sample obtained from a subject may include cells or cellular material (e.g., nucleic acids) of multiple organisms (e.g., virus nucleic acid, fetal nucleic acid, bacterial nucleic acid, parasite nucleic acid). A sample may include a cell and RNA transcripts. A sample can include nucleic acids obtained from one or more subjects. In some embodiments a sample includes nucleic acid obtained from a single subject. A subject can be any living or non-living organism, including but not limited to a human, non-human animal, plant, bacterium, fungus, virus, or protist. A subject may be any age (e.g., an embryo, a fetus, infant, child, adult). A subject can be of any sex (e.g., male, female, or combination thereof). A subject may be pregnant. In some embodiments, a subject is a mammal. In some embodiments, a subject is a plant. In some embodiments, a subject is a human subject. A subject can be a patient (e.g., a human patient). In some embodiments a subject is suspected of having a genetic variation or a disease or condition associated with a genetic variation.

As used herein, the term “disease state” is used in accordance with its plain and ordinary meaning and, in the context of the present disclosure, refers specifically to inflammatory bowel disease (IBD) and related pathological conditions of the gastrointestinal tract. A disease state may include ulcerative colitis, Crohn's disease, indeterminate colitis, and associated complications such as strictures, fistulas, fibrosis, and neoplasia. The presence of a disease state may be identified by the same collection of biological constituents used to determine the biological state of a cell or tissue, including nucleic acid molecules, proteins, and spatial patterns of expression within affected mucosa. In general, an IBD disease state is detrimental to the biological system of the gut, leading to chronic inflammation, architectural distortion of tissue, and disruption of barrier function. The IBD disease state can be a consequence of immune dysregulation, genetic predisposition, and environmental triggers, and is characterized by the aberrant interplay of epithelial cells, stromal cells, and multiple immune cell lineages, including T cells, B cells, macrophages, and dendritic cells. In some embodiments, the IBD disease state is associated with alterations in the expression of pro-inflammatory cytokines such as TNF, IL1B, IL6, IL17A, and IFNG, together with changes in anti-inflammatory mediators such as IL10 and FOXP3. Spatial clustering of such transcripts, particularly within colonic crypts, granulomas, Peyer's patches, or fibrotic lesions, provides characteristic molecular signatures of the disease. Disease states are monitored to determine the level or severity of IBD, including activity, extent, or progression of inflammation, and more specifically, to detect changes in the biological state of a subject that are correlated with therapeutic response or resistance. In embodiments, methods provided herein are applicable to monitoring the IBD disease state in subjects undergoing therapies, such as anti-TNF agents, IL-12/23 inhibitors, integrin blockers, or emerging biologic or small molecule interventions. Thus, the present disclosure also provides, in some embodiments, methods for determining or monitoring the efficacy of an IBD therapy (i.e., determining a level of therapeutic effect) upon a subject. These methods may be applied in clinical practice or in a clinical trial setting, serving as an early surrogate marker for therapeutic success or failure. Within inflamed tissues of IBD, there are hundreds to thousands of signaling pathways that are interconnected, and perturbations in the function of proteins or transcripts within a single pathway often produce compensatory changes in many others. Accordingly, the disruption or modulation of even a single gene or protein, whether by disease activity or therapeutic intervention, results in characteristic compensatory changes that can be detected at the transcript or protein level. These changes form a molecular “signature” of IBD or of a therapeutic response, which can be identified even at early stages where histological or endoscopic changes may not yet be apparent.

The terms “polypeptide,” “peptide” and “protein” are used interchangeably herein to refer to a polymer of amino acid residues, wherein the polymer may optionally be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymer. A protein may refer to a protein expressed in a cell.

A polypeptide, or a cell is “recombinant” when it is artificial or engineered, or derived from or contains an artificial or engineered protein or nucleic acid (e.g., non-natural or not wild type). For example, a polynucleotide that is inserted into a vector or any other heterologous location, e.g., in a genome of a recombinant organism, such that it is not associated with nucleotide sequences that normally flank the polynucleotide as it is found in nature is a recombinant polynucleotide. A protein expressed in vitro or in vivo from a recombinant polynucleotide is an example of a recombinant polypeptide. Likewise, a polynucleotide sequence that does not appear in nature, for example a variant of a naturally occurring gene, is recombinant.

As used herein, a “single cell” refers to one cell. Single cells useful in the methods described herein can be obtained from a tissue of interest, or from a biopsy, blood sample, or cell culture. Additionally, cells from specific organs, tissues, tumors, neoplasms, or the like can be obtained and used in the methods described herein. In general, cells from any population can be used in the methods, such as a population of prokaryotic or eukaryotic organisms, including bacteria or yeast.

The term “cellular component” is used in accordance with its ordinary meaning in the art and refers to any organelle, nucleic acid, protein, or analyte that is found in a prokaryotic, eukaryotic, archaeal, or other organismic cell type. Examples of cellular components (e.g., a component of a cell) include RNA transcripts, proteins, membranes, lipids, and other analytes.

A “gene” refers to a polynucleotide that is capable of conferring biological function after being transcribed and/or translated. A “gene” refers to a polynucleotide sequence that is capable of conferring biological function after being transcribed and/or translated. Functionally, a genome is subdivided into genes. Each gene is a nucleic acid sequence that encodes an RNA or polypeptide. A gene is transcribed from DNA into RNA, which can either be non-coding (ncRNA) with a direct function, or an intermediate messenger (mRNA) that is then translated into protein. Typically a gene includes multiple sequence elements, such as for example, a coding element (i.e., a sequence that encodes a functional protein), non-coding element, and regulatory element. Each element may be as short as a few bp to 5 kb. In embodiments, the gene is the protein coding sequence of RNA. Non-limiting examples of genes include developmental genes (e.g., adhesion molecules, cyclin kinase inhibitors, Wnt family members, Pax family members, Winged helix family members, Hox family members, cytokines/lymphokines and their receptors, growth/differentiation factors and their receptors, neurotransmitters and their receptors); oncogenes (e.g., ABL1, BCL1, BCL2, BCL6, CBFA2, CBL, CSF1R, ERBA, ERBB, ERBB2, ETS1, ETS1, ETV6, FGR, FOS, FYN, HCR, HRAS, JUN, KRAS, LCK, LYN, MDM2, MLL, MYB, MYC, MYCL1, MYCN, NRAS, PIM1, PML, RET, SRC, TAL1, TCL3, and YES); tumor suppressor genes (e.g., APC, BRCA1, BRCA2, MADH4, MCC, NF1, NF2, RB1, TP53, and WT1); and enzymes (e.g., ACC synthases and oxidases, ACP desaturases and hydroxylases, ADP-glucose pyrophorylases, ATPases, alcohol dehydrogenases, amylases, amyloglucosidases, catalases, cellulases, chalcone synthases, chitinases, cyclooxygenases, decarboxylases, dextrinases, DNA and RNA polymerases, galactosidases, glucanases, glucose oxidases, granule-bound starch synthases, GTPases, helicases, hemicellulases, integrases, inulinases, invertases, isomerases, kinases, lactases, lipases, lipoxygenases, lysozymes, nopaline synthases, octopine synthases, pectinesterases, peroxidases, phosphatases, phospholipases, phosphorylases, phytases, plant growth regulator synthases, polygalacturonases, proteinases and peptidases, pullanases, recombinases, reverse transcriptases, RUBISCOs, topoisomerases, and xylanases). In embodiments, a gene includes at least one mutation associated with a disease or condition mediated by a mutant form of the gene.

As used herein, the terms “biomolecule” or “analyte” refer to an agent (e.g., a compound, macromolecule, or small molecule), and the like derived from a biological system (e.g., an organism, a cell, or a tissue). The biomolecule may contain multiple individual components that collectively construct the biomolecule, for example, in embodiments, the biomolecule is a polynucleotide wherein the polynucleotide is composed of nucleotide monomers. The biomolecule may be or may include DNA, RNA, organelles, carbohydrates, lipids, proteins, or any combination thereof. These components may be extracellular. In some examples, the biomolecule may be referred to as a clump or aggregate of combinations of components. In some instances, the biomolecule may include one or more constituents of a cell but may not include other constituents of the cell. In embodiments, a biomolecule is a molecule produced by a biological system (e.g., an organism). The biomolecule may be any substance (e.g. molecule) or entity that is desired to be detected by the method of the invention. In embodiments, the biomolecule is the “target” of the assay methods described herein. The biomolecule may accordingly be any compound that may be desired to be detected, for example a peptide or protein, or nucleic acid molecule or a small molecule, including organic and inorganic molecules. The biomolecule may be a cell or a microorganism, including a virus, or a fragment or product thereof. Biomolecules of particular interest may thus include proteinaceous molecules such as peptides, polypeptides, proteins or prions or any molecule which includes a protein or polypeptide component, etc., or fragments thereof. The biomolecule may be a single molecule or a complex that contains two or more molecular subunits, which may or may not be be covalently bound to one another, and which may be the same or different. Thus, in addition to cells or microorganisms, such a complex biomolecule may also be a protein complex. Such a complex may thus be a homo- or hetero-multimer. Aggregates of molecules e.g., proteins may also be target analytes, for example aggregates of the same protein or different proteins. The biomolecule may also be a complex between proteins or peptides and nucleic acid molecules such as DNA or RNA. Of particular interest may be the interactions between proteins and nucleic acids, e.g., regulatory factors, such as transcription factors, and interactions between DNA or RNA molecules

As used herein, “biomaterial” refers to any biological material produced by an organism. In some embodiments, biomaterial includes secretions, extracellular matrix, proteins, lipids, organelles, membranes, cells, portions thereof, and combinations thereof. In some embodiments, cellular material includes secretions, extracellular matrix, proteins, lipids, organelles, membranes, cells, portions thereof, and combinations thereof. In some embodiments, biomaterial includes viruses. In some embodiments, the biomaterial is a replicating virus and thus includes virus infected cells. In embodiments, a biological sample includes biomaterials.

In some embodiments, a sample includes one or more nucleic acids, or fragments thereof. A sample can include nucleic acids obtained from one or more subjects. In some embodiments a sample includes nucleic acid obtained from a single subject. In some embodiments, a sample includes a mixture of nucleic acids. A mixture of nucleic acids can include two or more nucleic acid species having different nucleotide sequences, different fragment lengths, different origins (e.g., genomic origins, cell or tissue origins, subject origins, the like or combinations thereof), or combinations thereof. A sample may include synthetic nucleic acid.

A subject can be any living or non-living organism, including but not limited to a human, non-human animal, plant, bacterium, fungus, virus or protist. A subject may be any age (e.g., an embryo, a fetus, infant, child, adult). A subject can be of any sex (e.g., male, female, or combination thereof). A subject may be pregnant. In some embodiments, a subject is a mammal. In some embodiments, a subject is a human subject. A subject can be a patient (e.g., a human patient). In some embodiments a subject is suspected of having a genetic variation or a disease or condition associated with a genetic variation.

The methods and kits of the present disclosure may be applied, mutatis mutandis, to the sequencing of RNA, or to determining the identity of a ribonucleotide.

As used herein, the term “kit” refers to any delivery system for delivering materials. In the context of reaction assays, such delivery systems include systems that allow for the storage, transport, or delivery of reaction reagents (e.g., oligonucleotides, enzymes, etc. in the appropriate containers) and/or supporting materials (e.g., packaging, buffers, written instructions for performing a method, etc.) from one location to another. For example, kits include one or more enclosures (e.g., boxes) containing the relevant reaction reagents and/or supporting materials. As used herein, the term “fragmented kit” refers to a delivery system including two or more separate containers that each contain a subportion of the total kit components. The containers may be delivered to the intended recipient together or separately. For example, a first container may contain an enzyme for use in an assay, while a second container contains oligonucleotides. In contrast, a “combined kit” refers to a delivery system containing all of the components of a reaction assay in a single container (e.g., in a single box housing each of the desired components). The term “kit” includes both fragmented and combined kits.

As used herein the term “determine” can be used to refer to the act of ascertaining, establishing or estimating. A determination can be probabilistic. For example, a determination can have an apparent likelihood of at least 50%, 75%, 90%, 95%, 98%, 99%, 99.9% or higher. In some cases, a determination can have an apparent likelihood of 100%. An exemplary determination is a maximum likelihood analysis or report. As used herein, the term “identify,” when used in reference to a thing, can be used to refer to recognition of the thing, distinction of the thing from at least one other thing or categorization of the thing with at least one other thing. The recognition, distinction or categorization can be probabilistic. For example, a thing can be identified with an apparent likelihood of at least 50%, 75%, 90%, 95%, 98%, 99%, 99.9% or higher. A thing can be identified based on a result of a maximum likelihood analysis. In some cases, a thing can be identified with an apparent likelihood of 100%.

The terms “bioconjugate group,” “bioconjugate reactive moiety,” and “bioconjugate reactive group” refer to a chemical moiety which participates in a reaction to form a bioconjugate linker (e.g., covalent linker).

As used herein, the term “bioconjugate reactive moiety” and “bioconjugate reactive group” refers to a moiety or group capable of forming a bioconjugate (e.g., covalent linker) as a result of the association between atoms or molecules of bioconjugate reactive groups. The association can be direct or indirect. For example, a conjugate between a first bioconjugate reactive group (e.g., —NH₂, —COOH, —N-hydroxysuccinimide, or -maleimide) and a second bioconjugate reactive group (e.g., sulfhydryl, sulfur-containing amino acid, amine, amine sidechain containing amino acid, or carboxylate) provided herein can be direct, e.g., by covalent bond or linker (e.g., a first linker of second linker), or indirect, e.g., by non-covalent bond (e.g., electrostatic interactions (e.g., ionic bond, hydrogen bond, halogen bond), van der Waals interactions (e.g., dipole-dipole, dipole-induced dipole, London dispersion), ring stacking (pi effects), hydrophobic interactions and the like). In embodiments, bioconjugates or bioconjugate linkers are formed using bioconjugate chemistry (i.e., the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982. In embodiments, the first bioconjugate reactive group (e.g., maleimide moiety) is covalently attached to the second bioconjugate reactive group (e.g., a sulfhydryl). In embodiments, the first bioconjugate reactive group (e.g., haloacetyl moiety) is covalently attached to the second bioconjugate reactive group (e.g., a sulfhydryl). In embodiments, the first bioconjugate reactive group (e.g., pyridyl moiety) is covalently attached to the second bioconjugate reactive group (e.g., a sulfhydryl). In embodiments, the first bioconjugate reactive group (e.g., —N-hydroxysuccinimide moiety) is covalently attached to the second bioconjugate reactive group (e.g., an amine). In embodiments, the first bioconjugate reactive group (e.g., maleimide moiety) is covalently attached to the second bioconjugate reactive group (e.g., a sulfhydryl). In embodiments, the first bioconjugate reactive group (e.g., -sulfo-N-hydroxysuccinimide moiety) is covalently attached to the second bioconjugate reactive group (e.g., an amine).

Useful bioconjugate reactive groups used for bioconjugate chemistries herein include, for example: (a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters; (b) hydroxyl groups which can be converted to esters, ethers, aldehydes, etc.; (c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom; (d) dienophile groups which are capable of participating in Diels-Alder reactions such as, for example, maleimido or maleimide groups; (e) aldehyde or ketone groups such that subsequent derivatization is possible via formation of carbonyl derivatives such as, for example, imines, hydrazones, semicarbazones or oximes, or via such mechanisms as Grignard addition or alkyllithium addition; (f) sulfonyl halide groups for subsequent reaction with amines, for example, to form sulfonamides; (g) thiol groups, which can be converted to disulfides, reacted with acyl halides, or bonded to metals such as gold, or react with maleimides; (h) amine or sulfhydryl groups (e.g., present in cysteine), which can be, for example, acylated, alkylated or oxidized; (i) alkenes, which can undergo, for example, cycloadditions, acylation, Michael addition, etc.; (j) epoxides, which can react with, for example, amines and hydroxyl compounds; (k) phosphoramidites and other standard functional groups useful in nucleic acid synthesis; (l) metal silicon oxide bonding; (m) metal bonding to reactive phosphorus groups (e.g., phosphines) to form, for example, phosphate diester bonds; (n) azides coupled to alkynes using copper catalyzed cycloaddition click chemistry; (o) biotin conjugate can react with avidin or strepavidin to form a avidin-biotin complex or streptavidin-biotin complex.

An “antibody” (Ab) is a protein that binds specifically to a particular substance, known as an “antigen” (Ag). An “antibody” or “antigen-binding fragment” is an immunoglobulin that binds a specific “epitope.” The term encompasses polyclonal, monoclonal, and chimeric antibodies. In nature, antibodies are generally produced by lymphocytes in response to immune challenge, such as by infection or immunization. An “antigen” (Ag) is any substance that reacts specifically with antibodies or T lymphocytes (T cells). An antibody may include the entire antibody as well as any antibody fragments capable of binding the antigen or antigenic fragment of interest. Examples include complete antibody molecules, antibody fragments, such as Fab, F(ab′)2, CDRs, VL, VH, and any other portion of an antibody which is capable of specifically binding to an antigen. Antibodies used herein are immunospecific for, and therefore specifically and selectively bind to, for example, proteins either detected (e.g., biological targets of interest) or used for detection (e.g., probes containing oligonucleotide barcodes) in the methods and devices as described herein.

The term “covalent linker” is used in accordance with its ordinary meaning and refers to a divalent moiety which connects at least two moieties to form a molecule.

The term “non-covalent linker” is used in accordance with its ordinary meaning and refers to a divalent moiety which includes at least two molecules that are not covalently linked to each other but are capable of interacting with each other via a non-covalent bond (e.g., electrostatic interactions (e.g., ionic bond, hydrogen bond, halogen bond) or van der Waals interactions (e.g., dipole-dipole, dipole-induced dipole, London dispersion). In embodiments, the non-covalent linker is the result of two molecules that are not covalently linked to each other that interact with each other via a non-covalent bond.

As used herein a “genetically modifying agent” is a substance that alters the genetic sequence of a cell following exposure to the cell, resulting in an agent-mediated nucleic acid sequence. In embodiments, the genetically modifying agent is a small molecule, protein, pathogen (e.g., virus or bacterium), toxin, oligonucleotide, or antigen. In embodiments, the genetically modifying agent is a virus (e.g., influenza) and the agent-mediated nucleic acid sequence is the nucleic acid sequence that develops within a T-cell upon cellular exposure and contact with the virus. In embodiments, the genetically modifying agent modulates the expression of a nucleic acid sequence in a cell relative to a control (e.g., the absence of the genetically modifying agent).

Where a range of values is provided, it is understood that each intervening value, to the tenth of the unit of the lower limit unless the context clearly indicates otherwise, between the upper and lower limit of that range, and any other stated or unstated intervening value in, or smaller range of values within, that stated range is encompassed within the invention. The upper and lower limits of any such smaller range (within a more broadly recited range) may independently be included in the smaller ranges, or as particular values themselves, and are also encompassed within the invention, subject to any specifically excluded limit in the stated range. Where the stated range includes one or both of the limits, ranges excluding either or both of those included limits are also included in the invention.

As used herein, the term “upstream” refers to a region in the nucleic acid sequence that is towards the 5′ end of a particular reference point, and the term “downstream” refers to a region in the nucleic acid sequence that is toward the 3′ end of the reference point.

As used herein, the terms “incubate,” and “incubation refer collectively to altering the temperature of an object in a controlled manner such that conditions are sufficient for conducting the desired reaction. Thus, it is envisioned that the terms encompass heating a receptacle (e.g., a microplate) to a desired temperature and maintaining such temperature for a fixed time interval. Also included in the terms is the act of subjecting a receptacle to one or more heating and cooling cycles (i.e., “temperature cycling” or “thermal cycling”). While temperature cycling typically occurs at relatively high rates of change in temperature, the term is not limited thereto, and may encompass any rate of change in temperature.

As used herein, “biological activity” may include the in vivo activities of a compound or physiological responses that result upon in vivo administration of a compound, composition or other mixture. Biological activity, thus, may encompass therapeutic effects and pharmaceutical activity of such compounds, compositions and mixtures. Biological activities may be observed in vitro systems designed to test or use such activities.

The term “isolated” means altered or removed from the natural state. For example, a nucleic acid or a polypeptide naturally present in a living animal is not isolated, but the same nucleic acid or polypeptide partially or completely separated from the coexisting materials of its natural state is isolated. An isolated nucleic acid or protein can exist in substantially purified form, or can exist in a non-native environment such as, for example, a host cell. In embodiments, “isolated” refers to a nucleic acid, polynucleotide, polypeptide, protein, or other component that is partially or completely separated from components with which it is normally associated (other proteins, nucleic acids, cells, etc.).

The term “synthetic target” as used herein refers to a modified protein or nucleic acid such as those constructed by synthetic methods. In embodiments, a synthetic target is artificial or engineered, or derived from or contains an artificial or engineered protein or nucleic acid (e.g., non-natural or not wild type). For example, a polynucleotide that is inserted or removed such that it is not associated with nucleotide sequences that normally flank the polynucleotide as it is found in nature is a synthetic target polynucleotide.

The term “nucleic acid sequencing device” and the like means an integrated system of one or more chambers, ports, and channels that are interconnected and in fluid communication and designed for carrying out an analytical reaction or process, either alone or in cooperation with an appliance or instrument that provides support functions, such as sample introduction, fluid and/or reagent driving means, temperature control, detection systems, data collection and/or integration systems, for the purpose of determining the nucleic acid sequence of a template polynucleotide. Nucleic acid sequencing devices may further include valves, pumps, and specialized functional coatings on interior walls. Nucleic acid sequencing devices may include a receiving unit, or platen, that orients the flow cell such that a maximal surface area of the flow cell is available to be exposed to an optical lens. Other nucleic acid sequencing devices include those provided by Singular Genomics™ (e.g., the G4™ or G4X™ system), Illumina™ (e.g., HiSeq™, MiSeq™, NextSeq™, or NovaSeq™ systems), Life Technologies™ (e.g., ABI PRISM™, or SOLiD™ systems), Pacific Biosciences (e.g., systems using SMRT™ Technology such as the Sequel™ or RS II™ systems), or Qiagen (e.g., Genereader™ system). Nucleic acid sequencing devices may further include fluidic reservoirs (e.g., bottles), valves, pressure sources, pumps, sensors, control systems, valves, pumps, and specialized functional coatings on interior walls. In embodiments, the device includes a plurality of a sequencing reagent reservoirs and a plurality of clustering reagent reservoirs. In embodiments, the clustering reagent reservoir includes amplification reagents (e.g., an aqueous buffer containing enzymes, salts, and nucleotides, denaturants, crowding agents, etc.) In embodiments, the reservoirs include sequencing reagents (such as an aqueous buffer containing enzymes, salts, and nucleotides); a wash solution (an aqueous buffer); a cleave solution (an aqueous buffer containing a cleaving agent, such as a reducing agent); or a cleaning solution (a dilute bleach solution, dilute NaOH solution, dilute HCl solution, dilute antibacterial solution, or water). The fluid of each of the reservoirs can vary. The fluid can be, for example, an aqueous solution which may contain buffers (e.g., saline-sodium citrate (SSC), ascorbic acid, tris(hydroxymethyl)aminomethane or “Tris”), aqueous salts (e.g., KCl or (NH₄)₂SO₄)), nucleotides, polymerases, cleaving agent (e.g., tri-n-butyl-phosphine, triphenyl phosphine and its sulfonated versions (i.e., tris(3-sulfophenyl)-phosphine, TPPTS), and tri(carboxyethyl)phosphine (TCEP) and its salts, cleaving agent scavenger compounds (e.g., 2′-Dithiobisethanamine or 11-Azido-3,6,9-trioxaundecane-1-amine), chelating agents (e.g., EDTA), detergents, surfactants, crowding agents, or stabilizers (e.g., PEG, Tween, BSA). Non-limited examples of reservoirs include cartridges, pouches, vials, containers, and eppendorf tubes. In embodiments, the device is configured to perform fluorescent imaging. In embodiments, the device includes one or more light sources (e.g., one or more lasers). In embodiments, the illuminator or light source is a radiation source (i.e., an origin or generator of propagated electromagnetic energy) providing incident light to the sample. A radiation source can include an illumination source producing electromagnetic radiation in the ultraviolet (UV) range (about 200 to 390 nm), visible (VIS) range (about 390 to 770 nm), or infrared (IR) range (about 0.77 to 25 microns), or other range of the electromagnetic spectrum. In embodiments, the illuminator or light source is a lamp such as an arc lamp or quartz halogen lamp. In embodiments, the illuminator or light source is a coherent light source. In embodiments, the light source is a laser, LED (light emitting diode), a mercury or tungsten lamp, or a super-continuous diode. In embodiments, the light source provides excitation beams having a wavelength between 200 nm to 1500 nm. In embodiments, the laser provides excitation beams having a wavelength of 405 nm, 470 nm, 488 nm, 514 nm, 520 nm, 532 nm, 561 nm, 633 nm, 639 nm, 640 nm, 800 nm, 808 nm, 912 nm, 1024 nm, or 1500 nm. In embodiments, the illuminator or light source is a light-emitting diode (LED). The LED can be, for example, an Organic Light Emitting Diode (OLED), a Thin Film Electroluminescent Device (TFELD), or a Quantum dot based inorganic organic LED. The LED can include a phosphorescent OLED (PHOLED). In embodiments, the nucleic acid sequencing device includes an imaging system (e.g., an imaging system as described herein). The imaging system capable of exciting one or more of the identifiable labels (e.g., a fluorescent label) linked to a nucleotide and thereafter obtain image data for the identifiable labels. The image data (e.g., detection data) may be analyzed by another component within the device. The imaging system may include a system described herein and may include a fluorescence spectrophotometer including an objective lens and/or a solid-state imaging device. The solid-state imaging device may include a charge coupled device (CCD) and/or a complementary metal oxide semiconductor (CMOS). The system may also include circuitry and processors, including systems using microcontrollers, reduced instruction set computers (RISC), application specific integrated circuits (ASICs), field programmable gate array (FPGAs), logic circuits, and any other circuit or processor capable of executing functions described herein. The set of instructions may be in the form of a software program. As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. In embodiments, the device includes a thermal control assembly useful to control the temperature of the reagents.

The term “image” is used according to its ordinary meaning and refers to a representation of all or part of an object. The representation may be an optically detected reproduction. For example, an image can be obtained from fluorescent, luminescent, scatter, or absorption signals. The part of the object that is present in an image can be the surface or other xy plane of the object. Typically, an image is a 2 dimensional representation of a 3 dimensional object. An image may include signals at differing intensities (i.e., signal levels). An image can be provided in a computer readable format or medium. An image is derived from the collection of focus points of light rays coming from an object (e.g., the sample), which may be detected by any image sensor.

As used herein, the term “signal” is intended to include, for example, fluorescent, luminescent, scatter, or absorption impulse or electromagnetic wave transmitted or received. Signals can be detected in the ultraviolet (UV) range (about 200 to 390 nm), visible (VIS) range (about 391 to 770 nm), infrared (IR) range (about 0.771 to 25 microns), or other range of the electromagnetic spectrum. The term “signal level” refers to an amount or quantity of detected energy or coded information. For example, a signal may be quantified by its intensity, wavelength, energy, frequency, power, luminance, or a combination thereof. Other signals can be quantified according to characteristics such as voltage, current, electric field strength, magnetic field strength, frequency, power, temperature, etc. Absence of signal is understood to be a signal level of zero or a signal level that is not meaningfully distinguished from noise.

The term “xy coordinates” refers to information that specifies location, size, shape, and/or orientation in an xy plane. The information can be, for example, numerical coordinates in a Cartesian system. The coordinates can be provided relative to one or both of the x and y axes or can be provided relative to another location in the xy plane (e.g., a fiducial). The term “xy plane” refers to a 2 dimensional area defined by straight line axes x and y. When used in reference to a detecting apparatus and an object observed by the detector, the xy plane may be specified as being orthogonal to the direction of observation between the detector and object being detected.

As used herein, the term “tissue section” refers to a piece of tissue that has been obtained from a subject, optionally fixed and attached to a surface, e.g., a microscope slide.

As used herein, the term “data string” refers to a sequence of characters derived from a biological or computational data set, such as nucleotide sequences representing candidate probe segments. Each data string may comprise alphanumeric characters, symbols, or encoded representations corresponding to nucleotides or other sequence-based identifiers. The term “collection,” when used in reference to data strings, refers to one or more data strings grouped, organized, or processed together. A collection may comprise data strings derived from a single data set or from two or more distinct data sets. In one example, a collection of data strings may include candidate oligonucleotide sequences generated from exonic regions of a target gene. In another example, a collection may include sequences derived from multiple target genes or reference genomes. As used herein, a “plurality of data strings” refers to two or more data strings. Each data string may correspond to a candidate probe sequence comprising a first segment and a second segment, each segment having a length between 15 and 20 nucleotides, as described in the methods herein.

As used herein, a “subsequence”, “substring”, “prefix” or “suffix” of a string represents a subset of characters, letters, words, etc., of a longer list of characters, letters, words, etc., (i.e., the longer list being the sequence or string) wherein the order of the elements is preserved. A “prefix” typically refers to a subset of characters, letters, numbers, etc. found at the beginning of a sequence or string, whereas a “suffix” typically refers to a subset of characters, letters, numbers, etc. found at the end of a string. Substrings are also known as subwords or factors of a sequence or string.

As used herein, the term “weak base” refers to refers to adenine (A) and thymine (T) bases as these nitrogenous bases form two hydrogen bond pairs and thus weaker bonds compared to the guanine (G) or cytosine (C) bases pairs.

As used herein, the term “strong base” refers to guanine (G) or cytosine (C) bases as these nitrogenous bases form three hydrogen bond pairs and thus stronger bonds compared to the adenine (A) and thymine (T) base pairs.

As used herein, the term “predicted melting temperature” or “melting temperature” or “Tm” refers to a computationally estimated temperature at which 50% of an oligonucleotide is expected to dissociate from its complementary strand under hybridization conditions (e.g., oligonucleotide concentration, ionic strength (e.g., sodium or magnesium ion concentration), pH, and/or solvent environment). The predicted melting temperature is calculated using thermodynamic models that account for sequence-specific hybridization stability, including base pairing and nearest-neighbor stacking interactions.

The term “organelle” as used herein refers to an entity of cell associated with a particular function. In embodiments, an organelle refers to a specialized subunit within a cell that has a specific function, and is usually separately enclosed within its own lipid bilayer. Examples of organelles include the nucleus, mitochondria, endoplasmic reticulum, Golgi apparatus, lysosomes, and chloroplasts (in plant cells). Although most organelles are functional units within cells, some organelles function extend outside of cells, such as cilia, flagellum, archaellum, and the trichocyst. In embodiments, the organelle is a membrane bound organelle. In embodiments, the organelle is a non-membrane bound organelle. Non-membrane bounded organelles, also called biomolecular complexes, are assemblies of macromolecules such as the ribosome, the spliceosome, the proteasome, the nucleosome, and the centriole. Commonly detected organelles includes the nucleus, which is often visualized using dyes such as DAPI, Hoechst, and SYTO™ Green, mitochondria are with MitoTracker™ dyes and Rhodamine 123, endoplasmic reticulum (ER) utilizing dyes like ER-Tracker® Green/Red or DiOC6, the Golgi apparatus is stained with BODIPY™ FL C5-Ceramide and NBD C6-Ceramide, lysosomes are typically stained using LysoTracker™ dyes and Acridine Orange, and peroxisomes may be stained with Peroxisome-Tracker® Red and Peroxy Green dyes. Although not membrane-bound, ribosomes may detected using antibodies such as anti-RPL10 or anti-RPS6. Additionally, the cytoskeleton, specifically actin filaments, is frequently stained to study cell shape with Phalloidin conjugates and Alexa Fluor® Phalloidin being widely used. In embodiments, the organelle is a biomolecular complex including a plurality of subunits. In embodiments, the organelle is a macromolecule. In embodiments, the organelle is a eukaryotic organelle. In embodiments, the organelle is the cell membrane, the endoplasmic reticulum, a flagellum, a Golgi apparatus, a mitochondria, the nucleus, a vacuole. In embodiments, the organelle is a lysosome. In embodiments, the organelle is the nucleolus.

The term “conjugate” is used in its accordance with its plain and ordinary meaning and refers to a composition containing at least two components linked together. The individual components may be linked directly through one or more covalent bonds, or one or more ionic bonds, or by chelation, or mixtures thereof. The linkage, or conjugation, may include one or more spacer groups between the one or more linkages joining the one or more individual components, or may be between the individual component and the linkage. The individual components that may be linked together may include biologically derived biopolymers, modified biopolymers, biologically derived biomolecules, and synthetically derived molecules. For example, the conjugate may comprise a first component, such as a protein, that may be linked, i.e., conjugated, directly through one or more covalent bonds to a second component, such as an oligonucleotide, to form a conjugate. The conjugate and/or the linkage of the conjugate may be stable to thermolysis, stable to hydrolysis, may be biocompatible, or combinations thereof.

The term “computing device” is used herein to refer to an electronic device equipped with at least a processor. Examples of computing devices may include system or device described herein, mobile devices (e.g., cellular telephones, wearable devices, smartphones, smartwatches, web-pads, tablet computers, Internet enabled cellular telephones, Wi-Fi® enabled electronic devices, personal data assistants (PDAs), laptop computers, etc.), personal computers, and server computing devices. In various embodiments, computing devices may be configured with memory and/or storage as well as networking capabilities, such as network transceiver(s) and antenna(s) configured to establish a wide area network (WAN) connection (e.g., a cellular network connection, etc.) and/or a local area network (LAN) connection (e.g., a wired/wireless connection to the Internet via a Wi-Fi® router, etc.). In embodiments, the computing device is a mobile device, such as a cellular telephone, wearable device, or smartphone (e.g., iPhone, Android, Blackberry, Palm, Symbian, or Windows).

As used in this application, the terms “component”, “module”, “system”, and the like are intended to refer to a computer-related entity, either hardware, a combination of hardware and software, software, or software in execution. For example, a component may be, but is not limited to being, a process running on a processor, a processor, an object, an executable, a thread of execution, a program, and/or a computer. By way of illustration, both an application running on a server and the server can be a component. One or more components may reside within a process and/or thread of execution and a component may be localized on one computer and/or distributed between two or more computers.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### II. Compositions & Kits

In an aspect is provided a probe panel including a plurality of oligonucleotide probes as described herein (e.g., a sequence at least 80% homologous to SEQ ID NO:1 to SEQ ID NO:10530). In embodiments, each oligonucleotide probe includes a first hybridization sequence designed to specifically bind to a first target sequence of an RNA molecule and a second hybridization sequence designed to specifically bind to a second target sequence of the RNA molecule, wherein the probe panel is configured to detect at least 25 RNA molecules associated with IBD. For example, the probe panel may be configured to detect at least 25 RNA molecules selected from the group consisting of TNF, IL1B, IL6, IL17A, IL10, IL10RA, IL23R, IFNG, CXCL9, CXCL10, CXCL11, CXCR3, CCR7, CD3E, CD4, CD8A, FOXP3, CD68, MS4A1, ICOSLG, MUC2, CLDN1, EPCAM, CEACAM5, KRT20, COL1A1, TGFB1, and VCAM1. In embodiments, the probe panel is configured to detect a sequence selected from the group consisting of ACKR1, ACKR4, ACSS3, ACTA2, ADIPOQ, ANGPT2, ANKRD29, ANO1, ANXA1, ANXA13, APC, AQP1, AQP3, BEST2, BEST4, BRAF, CA2, CA7, CAMK2N1, CAVIN2, CCK, CCL2, CCL20, CCL3, CCL4, CCL5, CCR2, CCR5, CCR7, CD14, CD163, CD19, CD1C, CD2, CD247, CD27, CD274, CD276, CD28, CD33, CD34, CD36, CD38, CD3D, CD3E, CD4, CD40, CD40LG, CD44, CD47, CD68, CD70, CD74, CD79A, CD80, CD86, CD8A, CDC25C, CDH1, CDH19, CDX2, CEACAM1, CEACAM5, CEACAM8, CENPK, CHGA, CHRM2, CKB, CLCA1, CLCA4, CLDN1, CLDN3, CLDN4, CLEC9A, CLU, CNTNAP2, COL1A1, CPB1, CSF1R, CSPG4, CTLA4, CTNNA2, CTNNB1, CTSS, CX3CL1, CX3CR1, CXCL10, CXCL11, CXCL13, CXCL9, CXCR3, CXCR4, CXCR5, CXCR6, CYP1A1, CYP2A6, DDIT4, DEFA5, DES, DGKG, DPT, DSP, EGFR, ELF3, EOMES, EPCAM, EPHB3, ERBB2, ESM1, FABP1, FAM210B, FAP, FAS, FASN, FBLN1, FBN1, FCAR, FCGR1A, FCGR3A, FGB, FGFR4, FN1, FOXP3, FSCN1, FZD7, GATA3, GA™, GIP, GNLY, GPC1, GPR183, GPRC5A, GPX2, GREM1, GREM2, GRHL1, GUCA2A, GZMA, GZMB, GZMH, GZMK, HAVCR2, HLA-A, HLA-DRA, HNF4A, HOXD8, ICOSLG, ID2, IDO1, IER3, IFNG, IGFBP7, IGHA1, IGHD, IGHG1, IGHM, IL10, IL10RA, IL17A, IL18R1, IL1B, IL2RA, IL2RB, IL6, IL7R, INS, IRF1, ITGAM, ITGAX, ITGB2, ITLN1, JAK3, JCHAIN, KDR, KIT, KLF1, KLRB1, KLRD1, KLRF1, KLRK1, KRAS, KRT20, LAG3, LAMC3, LARS1, LGR5, LPL, LTBP2, LUM, LYVE1, LYZ, MADCAM1, MAPK1, MET, MKI67, MLN, MMP1, MMRN1, MMRN2, MRC1, MS4A1, MUC1, MUC12, MUC2, MUC5B, MUC6, MYC, MYH11, NCAM1, NCR1, NEUROD1, NEUROG3, NKG7, NONO, NOVA1, NRXN1, NTS, ODC1, OLFM4, PAX2, PDCD1, PDCD1LG2, PDE4A, PDGFRA, PDGFRB, PDK4, PECAMI, PHGR1, PIM1, PLAT, PLIN1, PLXND1, POLD2, POLR2A, PON2, POSTN, POU2AF1, POU2F3, PPARG, PRDM1, PRF1, PROM1, PROX1, PTGS2, PYGB, PYY, RARRES1, RBP2, REG1A, REG4, RGMB, RGS5, ROBO1, ROBO2, RRM2, RSPO3, RUNX1, S100A9, S100P, SCGN, SDC1, SELE, SELL, SETD5, SH2D6, SKA3, SLC16A1, SLC2A1, SLC3A2, SLC6A19, SLC7A5, SMOC2, SNCA, SNCG, SOX10, SPINK4, SST, ST14, STAT1, STAT3, STAT4, STC1, SYTL2, TAGLN, TAP1, TAP2, TBX21, TCF7, TCL1A, TFF3, TFP1, TGFB1, THBS1, THY1, TICRR, TIGIT, TLR2, TLR4, TLR9, TNF, TNFRSF17, TNFRSF4, TNFRSF9, TNFSF13B, TNFSF9, TOP2A, TP53, TPH1, TSPAN8, VCAM1, VCAN, VEGFA, VIM, VWF, WARS1, WNT2B, WNT5B, and ZNF800. In embodiments, each RNA molecule is detected by at least three oligonucleotide probes targeting distinct sequences of the RNA molecule.

In embodiments, the first and second hybridization sequences overlap by no more than 8 base pairs with each other. In embodiments, each probe sequence includes a sequence overlap of less than eight nucleotides between the first segment and the second segment of the same probe sequence. The first and second segments correspond to the two hybridization arms of a circularizable oligonucleotide probe. Limiting the overlap between these segments ensures that each segment binds to a distinct region of the target sequence, thereby preserving the specificity of hybridization. Overlap of greater than or equal to eight nucleotides may result in undesirable intra-molecular interactions, such as the formation of secondary structures or partial self-annealing, which may interfere with the hybridization, ligation, or amplification steps of the assay. The restriction on segment overlap reduces the likelihood of internal complementarity that could compromise probe performance, and facilitates the design of structurally stable probes suitable for multiplexed applications. In embodiments, the overlap constraint is applied during the computational design process and serves as an exclusion criterion when selecting candidate probes. The constraint supports the generation of probe sequences that retain structural distinction between their hybridization arms and minimizes sequence redundancy within each probe. In embodiments, the method includes removing any probe sequence that overlaps with another probe sequence in the second subset.

In an aspect is provided a solid support including a plurality of discrete tissue samples obtained from a patient having or suspected of having inflammatory bowel disease (IBD), wherein each tissue sample is selected from colon tissue, rectal tissue, ileal tissue, small intestine tissue, gastric tissue, esophageal tissue, perianal tissue, fibrotic tissue, or granulomatous tissue;

“\*MERGEFORMAT\* MERGEFORMAT wherein each tissue sample includes: (i) an RNA molecule hybridized to an oligonucleotide probe (e.g., a circularizable or circularized probe as described herein), the RNA molecule including a sequence of an IBD-associated gene; and (ii) a protein bound to a specific binding reagent, the protein being an IBD-associated protein. In embodiments, the IBD-associated gene is selected from TNF, IL1B, IL6, IL17A, CXCL9, CXCL10, or CXCL11. In embodiments, the IBD-associated protein is selected from CD68, FOXP3, CD4, CD8, or PD-1. In embodiments, the specific binding reagent includes an antibody covalently attached to a DNA oligonucleotide. In embodiments, the RNA molecule and the protein are co-localized within a segmented single cell of the tissue. In embodiments, the plurality of discrete tissue samples include both inflamed and non-inflamed mucosa from the same patient. In embodiments, the tissue is human tissue.

In embodiments, the RNA molecule includes a sequence of a gene selected from the group consisting of TNF, IL1B, IL6, IL17A, IFNG, IL23R, IL10, IL10RA, FOXP3, CXCL9, CXCL10, CXCL11, CXCR3, CCR7, MUC2, CLDN1, EPCAM, CEACAM5, KRT20, COL1A1, TGFB1, and VCAM1. In embodiments, the protein is selected from the group consisting of CD3E, CD4, CD8A, FOXP3, CD68, ITGAX, ITGAM, MS4A1, EPCAM, MUC2, PDCD1, and VCAM1.

In an aspect, provided herein are kits for use in accordance with any of the compounds, compositions, or methods disclosed herein, and including one or more elements thereof. In embodiments, a kit includes labeled nucleotides including differently labeled nucleotides, enzymes, buffers, oligonucleotides, and related solvents and solutions. In embodiments, the kit includes one or more oligonucleotide probes (e.g., an oligonucleotide probe as described herein). The kit may also include a template nucleic acid (DNA and/or RNA), one or more primer polynucleotides, nucleoside triphosphates (including, e.g., deoxyribonucleotides, dideoxynucleotides, ribonucleotides, labeled nucleotides, and/or modified nucleotides), buffers, salts, and/or labels (e.g., fluorophores). In embodiments, the kit includes components useful for circularizing template polynucleotides using a ligation enzyme (e.g., Circligase enzyme, Taq DNA Ligase, HiFi Taq DNA Ligase, T4 ligase, SplintR ligase, or Ampligase DNA Ligase). For example, such a kit further includes the following components: (a) reaction buffer for controlling pH and providing an optimized salt composition for a ligation enzyme (e.g., Circligase enzyme, Taq DNA Ligase, HiFi Taq DNA Ligase, T4 ligase, SplintR ligase, or Ampligase DNA Ligase), and (b) ligation enzyme cofactors. In embodiments, the kit further includes instructions for use thereof. In embodiments, kits described herein include a polymerase. In embodiments, the polymerase is a DNA polymerase. In embodiments, the DNA polymerase is a thermophilic nucleic acid polymerase. In embodiments, the DNA polymerase is a modified archaeal DNA polymerase. In embodiments, the kit includes a sequencing solution. In embodiments, the sequencing solution includes labeled nucleotides including differently labeled nucleotides, wherein the label (or lack thereof) identifies the type of nucleotide. For example, each adenine nucleotide, or analog thereof; a thymine nucleotide; a cytosine nucleotide, or analog thereof; and a guanine nucleotide, or analog thereof may be labeled with a different fluorescent label.

In an aspect is provided a plurality of oligonucleotide probes. In embodiments, the oligonucleotide probes are designed to specifically bind to a sequence of a nucleic acid molecule (e.g., an mRNA molecule). In embodiments, the oligonucleotide probe is designed to bind to a sequence of a nucleic acid molecule including a gene. In embodiments, the gene is ACKR1, ACKR4, ACSS3, ACTA2, ADIPOQ, ANGPT2, ANKRD29, ANO1, ANXA1, ANXA13, APC, AQP1, AQP3, BEST2, BEST4, BRAF, CA2, CA7, CAMK2N1, CAVIN2, CCK, CCL2, CCL20, CCL3, CCL4, CCL5, CCR2, CCR5, CCR7, CD14, CD163, CD19, CD1C, CD2, CD247, CD27, CD274, CD276, CD28, CD33, CD34, CD36, CD38, CD3D, CD3E, CD4, CD40, CD40LG, CD44, CD47, CD68, CD70, CD74, CD79A, CD80, CD86, CD8A, CDC25C, CDH1, CDH19, CDX2, CEACAM1, CEACAM5, CEACAM8, CENPK, CHGA, CHRM2, CKB, CLCA1, CLCA4, CLDN1, CLDN3, CLDN4, CLEC9A, CLU, CNTNAP2, COL1A1, CPB1, CSF1R, CSPG4, CTLA4, CTNNA2, CTNNB1, CTSS, CX3CL1, CX3CR1, CXCL10, CXCL11, CXCL13, CXCL9, CXCR3, CXCR4, CXCR5, CXCR6, CYP1A1, CYP2A6, DDIT4, DEFA5, DES, DGKG, DPT, DSP, EGFR, ELF3, EOMES, EPCAM, EPHB3, ERBB2, ESM1, FABP1, FAM210B, FAP, FAS, FASN, FBLN1, FBN1, FCAR, FCGR1A, FCGR3A, FGB, FGFR4, FN1, FOXP3, FSCN1, FZD7, GATA3, GA™, GIP, GNLY, GPC1, GPR183, GPRC5A, GPX2, GREM1, GREM2, GRHL1, GUCA2A, GZMA, GZMB, GZMH, GZMK, HAVCR2, HLA-A, HLA-DRA, HNF4A, HOXD8, ICOSLG, ID2, IDO1, IER3, IFNG, IGFBP7, IGHA1, IGHD, IGHG1, IGHM, IL10, IL10RA, IL17A, IL18R1, IL1B, IL2RA, IL2RB, IL6, IL7R, INS, IRF1, ITGAM, ITGAX, ITGB2, ITLN1, JAK3, JCHAIN, KDR, KIT, KLF1, KLRB1, KLRD1, KLRF1, KLRK1, KRAS, KRT20, LAG3, LAMC3, LARS1, LGR5, LPL, LTBP2, LUM, LYVE1, LYZ, MADCAM1, MAPK1, MET, MKI67, MLN, MMP1, MMRN1, MMRN2, MRC1, MS4A1, MUC1, MUC12, MUC2, MUC5B, MUC6, MYC, MYH11, NCAM1, NCR1, NEUROD1, NEUROG3, NKG7, NONO, NOVA1, NRXN1, NTS, ODC1, OLFM4, PAX2, PDCD1, PDCD1LG2, PDE4A, PDGFRA, PDGFRB, PDK4, PECAMI, PHGR1, PIM1, PLAT, PLIN1, PLXND1, POLD2, POLR2A, PON2, POSTN, POU2AF1, POU2F3, PPARG, PRDM1, PRF1, PROM1, PROX1, PTGS2, PYGB, PYY, RARRES1, RBP2, REG1A, REG4, RGMB, RGS5, ROBO1, ROBO2, RRM2, RSPO3, RUNX1, S100A9, S100P, SCGN, SDC1, SELE, SELL, SETD5, SH2D6, SKA3, SLC16A1, SLC2A1, SLC3A2, SLC6A19, SLC7A5, SMOC2, SNCA, SNCG, SOX10, SPINK4, SST, ST14, STAT1, STAT3, STAT4, STC1, SYTL2, TAGLN, TAP1, TAP2, TBX21, TCF7, TCL1A, TFF3, TFP1, TGFB1, THBS1, THY1, TICRR, TIGIT, TLR2, TLR4, TLR9, TNF, TNFRSF17, TNFRSF4, TNFRSF9, TNFSF13B, TNFSF9, TOP2A, TP53, TPH1, TSPAN8, VCAM1, VCAN, VEGFA, VIM, VWF, WARS1, WNT2B, WNT5B, or ZNF800. In embodiments, the oligonucleotide probe includes a sequence selected from SEQ ID NO:7021 to SEQ ID NO:10530.

In an aspect is provided an oligonucleotide probe including a sequence at least 80% homologous to SEQ ID NO:1 to SEQ ID NO:10530. In embodiments, the oligonucleotide probe includes a sequence 90% homologous to SEQ ID NO:1 to SEQ ID NO:10530. In embodiments, the oligonucleotide probe includes a sequence selected from SEQ ID NO:1 to SEQ ID NO:10530. In embodiments, the oligonucleotide probe includes a sequence selected from SEQ ID NO:7021 to SEQ ID NO:10530. In embodiments, the oligonucleotide probe includes a first sequence selected from SEQ ID NO:1 to SEQ ID NO:3510 and a second sequence selected from SEQ ID NO:3511 to SEQ ID NO:7020. In embodiments, the oligonucleotide probe includes a primer binding sequence, or complement thereof, selected from the following: SEQ ID NO:10531, SEQ ID NO:10532, SEQ ID NO:10533, SEQ ID NO:10534, SEQ ID NO:10535, SEQ ID NO:10536, SEQ ID NO:10537, SEQ ID NO:10538, SEQ ID NO:10539, SEQ ID NO:10540, SEQ ID NO:10541, SEQ ID NO:10542, SEQ ID NO:10543, or SEQ ID NO:10544.

In an aspect is provided a plurality of oligonucleotide probes, wherein each oligonucleotide probe includes a first end, a backbone sequence, and a second end, wherein the first end includes a sequence selected from SEQ ID NO:1 to SEQ ID NO:3510 and the second end includes a sequence selected from SEQ ID NO:3511 to SEQ ID NO:7020. In embodiments, the oligonucleotide probe is in a cell or tissue. In embodiments, the cell or tissue further includes a concatemer including multiple complementary copies of the oligonucleotide probe. In embodiments, the cell or tissue is immobilized to a solid support.

A probe is a molecule designed to recognize (and bind or hybridize to) another molecule, e.g., a target analyte, another probe molecule, etc. As used herein, the term “probe” may refer either to a chemical/physical probe molecule (e.g., a nucleic acid probe molecule) or to its representation in a computer-readable, digital format (e.g., as a string of characters representing the sequence of bases in a nucleic acid probe molecule). In embodiments, the oligonucleotide probe is a linear nucleic acid molecule. Exemplary probes or probe sets may be based on a padlock probe, a gapped padlock probe, a SNAIL (Splint Nucleotide Assisted Intramolecular Ligation) probe set, a PLAYR (Proximity Ligation Assay for RNA) probe set, a PLISH (Proximity Ligation in situ Hybridization) probe set, and RNA-templated ligation probes (e.g., including 1 to 4 ribonucleotides near the ligation junction). The specific probe or probe set design can vary. In some embodiments, the probes, such as a padlock probe or a probe set that comprises a padlock probe, contain one or more barcodes in the backbone sequence. In some embodiments, one or more barcodes are indicative of a sequence in the analyte nucleic acid, such as a single nucleotide (e.g., SNPs or point mutations), a dinucleotide sequence, a short sequence of about 5 nucleotides in length, or a sequence of any suitable length.

In embodiments, the oligonucleotide probe is about 50 to about 500 nucleotides in length. In embodiments, the oligonucleotide probe is about 50 to about 300 nucleotides in length. In embodiments, the oligonucleotide probe is about 80 to about 300 nucleotides in length. In embodiments, the oligonucleotide probe is about 50 to about 150 nucleotides in length. In embodiments, the oligonucleotide probe is about or more than about 40, 50, 75, 100, 125, 150, 175, 200, 250, 300, 350, 400, or 500 nucleotides in length. In embodiments, the oligonucleotide probe is less than about 50, 75, 100, 125, 150, 175, 200, 250, 300, 350, 400, or 500 nucleotides in length. In embodiments, each probe oligonucleotide (e.g., each probe oligonucleotide of a plurality of probe oligonucleotides) includes a primer binding sequence (i.e., a sequence complementary to a primer, such as an amplification or sequencing primer). In embodiments, the oligonucleotide probe is about 80 to about 90 nucleotides in length. In embodiments, the oligonucleotide probe is 80 to 100 nucleotides in length. In embodiments, the oligonucleotide probe is 80, 81, 82, 83, 84, 85, 86, 87, 88, 89, or 90 nucleotides. In embodiments, the oligonucleotide probe is 80 nucleotides. In embodiments, the oligonucleotide probe is 81 nucleotides. In embodiments, the oligonucleotide probe is 82 nucleotides. In embodiments, the oligonucleotide probe is 83 nucleotides. In embodiments, the oligonucleotide probe is 84 nucleotides. In embodiments, the oligonucleotide probe is 85 nucleotides. In embodiments, the oligonucleotide probe is 86 nucleotides. In embodiments, the oligonucleotide probe is 87 nucleotides. In embodiments, the oligonucleotide probe is 88 nucleotides. In embodiments, the oligonucleotide probe is 89 nucleotides. In embodiments, the oligonucleotide probe is 90 nucleotides.

In embodiments, the oligonucleotide probe includes a target hybridization sequence (i.e., a sequence complementary to a sequence of the target nucleic acid molecule). In embodiments, the target hybridization sequence includes 5 to 25 nucleotides. In embodiments, the target hybridization sequence is about 5 to about 35 nucleotides in length. In embodiments, the target hybridization sequence is about 12 to 15 nucleotides in length. In embodiments, the target hybridization sequence is about 15 to 30 nucleotides in length. In embodiments, the target hybridization sequence (e.g., the first and/or second target hybridization sequence) is greater than 30 nucleotides. In embodiments, the target hybridization sequence is about 5 to about 35 nucleotides in length. In embodiments, the target hybridization sequence is about 12 to 15 nucleotides in length. In embodiments, the target hybridization sequence is about 35 to 40 nucleotides in length to maximize specificity. In embodiments, the target hybridization sequence is greater than 12 nucleotides in length. In embodiments, the target hybridization sequence is 15 nucleotides in length. In embodiments, the target hybridization sequence is 20 nucleotides in length. In embodiments, the target hybridization sequence is 25 nucleotides in length. In embodiments, the target hybridization sequence is about 5, about 10, about 15, about 20, about 25, about 30, or about 35 nucleotides in length. In embodiments, the target hybridization sequence is about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In embodiments, the target hybridization sequence of each oligonucleotide primer is a single stranded polynucleotide that is at least 50% complementary, at least 75% complementary, at least 85% complementary, at least 90% complementary, at least 95% complementary, at least 98%, at least 99% complementary, or 100% complementary to a portion of a target polynucleotide. In embodiments, the target hybridization sequence includes about 45% to 65% GC content (i.e., the percentage of nucleobases that are either guanine or cytosine). In embodiments, the target hybridization sequence does not include 4 or more guanine or cytosine nucleobases. In embodiments, the target hybridization sequence is designed according to the methods and systems described herein.

In embodiments, the oligonucleotide does not include five consecutive weak bases. In embodiments, the oligonucleotide does not include five consecutive strong bases. In embodiments, the oligonucleotide does not include secondary structure. In embodiments, the oligonucleotide does not include 2, 3, 4, 5, 6, 7, 8, 9, or 10 consecutive weak bases. In embodiments, the oligonucleotide does not include 2, 3, 4, 5, 6, 7, 8, 9, or 10 consecutive strong bases. In embodiments, the oligonucleotide does not include 2, 3, 4, 5, 6, 7, 8, 9, or 10 consecutive weak bases or 2, 3, 4, 5, 6, 7, 8, 9, or 10 consecutive strong bases. In embodiments, the oligonucleotide does not include 2, 3, 4, 5, 6, 7, 8, 9, or 10 consecutive weak bases and does not include 2, 3, 4, 5, 6, 7, 8, 9, or 10 consecutive strong bases.

In embodiments, the plurality of probes is ordered into subsets. In embodiments, the probes in a probe subset have a similar melting temperature for binding to their target sites. For example, the Tm may range from about 45° C. to about 65° C., including any Tm within this range such as 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., or 65° C. In embodiments, the subset of oligonucleotide probes is grouped according to their sequencing primer sequence. For example, a first subset includes the sequencing primer sequence SEQ ID NO:10537 and the subset includes 663 oligonucleotide probes. The second subset includes the sequencing primer sequence SEQ ID NO:10538 and the subset includes 531 oligonucleotide probes. In embodiments, the plurality is divided into 7 subsets and grouped according to a sequencing primer sequence In embodiments, the probes of the first set are capable of binding between 75 and 150 different gene sequences. In embodiments, the probes are capable of binding between 25 and 75 different gene sequences. In embodiments, the probes are capable of binding between 30 and 100 different gene sequences. In embodiments, the probes are capable of binding between 40 and 120 different gene sequences. In embodiments, the probes are capable of binding between 50 and 150 different gene sequences. In embodiments, the probes are capable of binding between 60 and 180 different gene sequences. In embodiments, the probes are capable of binding between 70 and 200 different gene sequences. In embodiments, the probes are capable of binding between 80 and 220 different gene sequences. In embodiments, the probes are capable of binding between 90 and 250 different gene sequences. In embodiments, the probes are capable of binding between 100 and 300 different gene sequences. In embodiments, the probes are capable of binding between 150 and 400 different gene sequences.

In embodiments, the first probe set includes a plurality of oligonucleotide probes, wherein a first subset of the plurality of oligonucleotide probes includes a first sequencing primer binding sequence. A second subset includes a plurality of oligonucleotide probes, wherein each of the oligonucleotide probes includes the second sequencing primer binding sequence. A third subset includes a plurality of oligonucleotide probes, wherein each of the oligonucleotide probes includes the third sequencing primer binding sequence. A fourth subset includes a plurality of oligonucleotide probes, wherein each of the oligonucleotide probes includes the fourth sequencing primer binding sequence. A fifth subset includes a plurality of oligonucleotide probes, wherein each of the oligonucleotide probes includes the fifth sequencing primer binding sequence. A sixth subset includes a plurality of oligonucleotide probes, wherein each of the oligonucleotide probes includes the sixth sequencing primer binding sequence. A seventh subset includes a plurality of oligonucleotide probes, wherein each of the oligonucleotide probes includes the seventh sequencing primer binding sequence. An eighth subset includes a plurality of oligonucleotide probes, wherein each of the oligonucleotide probes includes the eighth sequencing primer binding sequence. A ninth subset includes a plurality of oligonucleotide probes, wherein each of the oligonucleotide probes includes the ninth sequencing primer binding sequence. A tenth subset includes a plurality of oligonucleotide probes, wherein each of the oligonucleotide probes includes the tenth sequencing primer binding sequence. An eleventh subset includes a plurality of oligonucleotide probes, wherein each of the oligonucleotide probes includes the eleventh sequencing primer binding sequence. A twelfth subset includes a plurality of oligonucleotide probes, wherein each of the oligonucleotide probes includes the twelfth sequencing primer binding sequence.

The probe sequences described herein may be screened to avoid common six-base-cutter restriction enzyme recognition sites to aid in the ease of manipulation for conventional molecular cloning techniques. Selected sequences are additionally subjected to predicted secondary structure analysis, and those with the least secondary structure are chosen for further evaluation. Any program known in the art can be used to predict secondary structure, such as the MFOLD program (Zuker, 2003, Nucleic Acids Res. 31 (13):3406-15; Mathews et al., 1999, J. Mol. Biol. 288:911-940).

In embodiments, the probe oligonucleotide includes one or more modifications, for example the oligonucleotide may include nucleotides modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids (see Hyrup et al., 1996, Bioorganic & Medicinal Chemistry 4(1):5-23). As used herein, the terms “peptide nucleic acids” (PNAs) refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength.

### III. Methods

In an aspect is provided a method of detecting, diagnosing, and/or treating Inflammatory Bowel Disease (IBD). When studying IBD, particularly conditions like ulcerative colitis (UC) and Crohn's disease, several tissue types are critical for comprehensive analysis. Colon tissue, including both inflamed and non-inflamed mucosa, is essential for understanding disease activity and treatment effectiveness, while normal adjacent tissue allows for comparisons that reveal the extent of inflammation and damage. Rectal tissue is particularly relevant in UC, as inflammation often begins in the rectum and progresses proximally. Ileal tissue is important in Crohn's disease, especially when the disease affects the terminal ileum, and small intestine tissue is also valuable for studying Crohn's disease, which can involve any part of the gastrointestinal (GI) tract. Lymphoid tissues, such as Peyer's patches in the small intestine and mesenteric lymph nodes, are crucial for exploring the immune system's role in IBD. Gastric tissue can be relevant in Crohn's disease cases where inflammation extends to the stomach, while esophageal tissue, although rarely involved, is pertinent when Crohn's disease affects the esophagus. Perianal tissue is particularly useful in studying perianal Crohn's disease, which is associated with complications like abscesses and fistulas. Fibrotic tissue, commonly found in chronic IBD cases, is essential for understanding complications such as strictures, while granulomatous tissue, specific to Crohn's disease, serves as a hallmark for differentiating it from UC. Additionally, neoplastic tissue, including dysplasia or cancer, is important for studying the increased risk of colorectal cancer in long-standing IBD. Biopsies from fistulas provide insight into disease severity and progression in Crohn's disease, and surgical resection samples offer a comprehensive view of disease extent, severity, and response to therapy.

In an aspect is provided a method of detecting a biomolecule in or on a cell or tissue. In embodiments, the method includes immobilizing a cell or tissue including a biomolecule to a solid support; contacting the biomolecule in or on the cell or tissue with an oligonucleotide as described herein, binding a detection agent (e.g., a probe) including a label to the probe oligonucleotide (or an amplification product thereof); detecting the label, thereby detecting the biomolecule. For example, the method includes delivering antibody-oligonucleotide (Ab-oligo) conjugates (e.g., a specific binding agent described herein) and subsequent amplification probes, such as padlock probes (e.g., a polynucleotide probe described herein), bound to the oligonucleotide portion of the Ab-oligo). In embodiments, the method includes imaging the tissue section. In embodiments, the detection agent is a biomolecule-specific binding agent. In embodiments, the biomolecule-specific binding agent is a protein-specific binding agent. In embodiments, the biomolecule-specific binding agent is an oligonucleotide-specific binding agent. In embodiments, the biomolecule-specific binding agent is capable of binding to a cluster of differentiation (CD) marker, integrin, selectin, cadherin, cytokine receptor, chemokine receptor, Toll-like receptor (TLR), ion channel, transmembrane protein, lipoprotein, glycoprotein, cell surface protein, transport protein, intracellular organelle, or transcription factor. In embodiments, the intracellular organelle includes actin, carbohydrate, centrosomes and centrioles, chloroplasts (in plant cells and some protists), cytoskeleton, endoplasmic reticulum, endosome, Golgi apparatus, intermediate filaments, lysosome, microfilaments, microtubules, mitochondria, nuclear envelope, nuclear pores, nucleoid, nucleolus, nucleus, peroxisome, phosphatidylserine, plasma membrane, ribosomes, rough endoplasmic reticulum, smooth endoplasmic reticulum, transferrin receptor, transport vesicles, and/or vacuoles. In embodiments, the biomolecule specific binding agent is capable of binding to a biomolecule in the mitogen-activated protein kinase (MAPK) pathway, PI3K/AKT/mTOR pathway, Wnt/β-catenin pathway, intrinsic (mitochondrial) pathway, extrinsic (death receptor) pathway, caspase cascade, Notch signaling pathway, hedgehog signaling pathway, TGF-β (transforming growth factor Beta) pathway, JAK/STAT pathway, G-protein coupled receptor (GPCR) pathway, calcium signaling pathway, glycolysis, citric acid cycle (Krebs Cycle), oxidative phosphorylation, lipid metabolism pathway, amino acid metabolism, Toll-like receptor (TLR) pathway, NF-κB signaling pathway, complement pathway, nucleotide excision repair (NER), base excision repair (BER), mismatch repair (MMR), cyclin-dependent kinase (CDK) pathway, Rb (retinoblastoma) pathway, p53 pathway, unfolded protein response (UPR), heat shock response pathway, oxidative stress pathway, BMP (bone morphogenetic protein) pathway, FGF (fibroblast growth factor) pathway, Sonic Hedgehog pathway, neurotrophin signaling pathway, synaptic transmission pathway, axon guidance pathways, insulin signaling pathway, thyroid hormone pathway, steroid hormone pathway, VEGF (vascular endothelial growth factor) pathway, DNA methylation pathway, histone modification pathway, or angiogenesis. In embodiments, the biomolecule specific binding agent is capable of binding to a biomolecule on the surface of or in a B cell, Mature B Cell, Follicular B cell, Marginal Zone B cell, Short lived plasma cell, Memory B cell, Long lived plasma cell, B1 cell, Breg, Germinal Center B cell, Macrophage, Monocyte, M1 macrophage, M2 macrophage, Dendritic Cell, Plasmacytoid dendritic cell, Monocyte-derived dendritic cell, T cell, T Follicular Helper, Th1, Th2, Th9, Th17, Th22, Treg, platelet (activated), platelet (rested), natural killer cell, neutrophil, basophil, eosinophil, mast cell, astrocyte, neuron, glial cell, lymphocyte, myeloid cell, granulocytes, neural cells, stem cells, endothelial cells, epithelial cells, mesenchymal stem cell, hematopoietic stem cell, embryonic stem, stromal cell, erythrocyte, fibroblast, or apoptotic cell.

In embodiments, the detection agent is an oligonucleotide-specific binding agent capable of hybridizing to a target oligonucleotide sequence in a tissue section. In embodiments, the detection agent is an oligonucleotide. In embodiments, the detection agent is an oligonucleotide, wherein the oligonucleotide includes: a) a first region at a 3′ end that is hybridized to a first complementary region of the polynucleotide, and b) a second region at a 5′ end that is hybridized to a second complementary region of the polynucleotide, wherein the second complementary region is 5′ with respect to the first complementary region. In embodiments, the method includes i) circularizing the oligonucleotide agent to generate a circular oligonucleotide and ligating the oligonucleotide-specific binding agent; ii) amplifying the circular oligonucleotide by extending an amplification primer hybridized to the circular oligonucleotide with a strand-displacing polymerase, wherein the amplification primer extension generates an extension product including multiple complements of the circular oligonucleotide; and iii) sequencing the extension product of step (ii). In embodiments, circularizing the oligonucleotide-specific binding agent includes extending the 3′ end of the oligonucleotide-specific binding agent (using a polymerase to incorporate one or more nucleotides) along the target nucleic acid to generate a complementary sequence and ligating the extended 3′ end of the oligonucleotide-specific binding agent to the 5′ end of the oligonucleotide-specific binding agent. In embodiments, the circular oligonucleotide includes a barcode sequence. In embodiments, circularizing in step i) further includes extending the 3′ end of the oligonucleotide primer (e.g., extending the 3′ end of the primer using a polymerase (e.g., a Thermus thermophilus (Tth) DNA polymerase) to incorporate one or more nucleotides) along the target nucleic acid to generate a complementary sequence (e.g., complementary to the target nucleic acid, for example a target RNA sequence) prior to ligating the complementary sequence to the 5′ end of the oligonucleotide primer. In embodiments, the oligonucleotide is an oligonucleotide primer.

In an aspect is provided a method of detecting nucleic acid molecules in a tissue sample from a patient having or suspected of having inflammatory bowel disease (IBD), the method including: i) contacting the tissue sample with a first polynucleotide probe and binding the first polynucleotide probe to a first nucleic acid molecule, and contacting the sample with a second polynucleotide probe and binding the second polynucleotide probe to a second nucleic acid molecule, wherein the first polynucleotide probe includes a first oligonucleotide binding sequence and a first sequence, and wherein the second polynucleotide probe includes a second oligonucleotide binding sequence and a second sequence; ii) amplifying the first and second polynucleotide probes to generate amplification products; iii) hybridizing a first oligonucleotide to the first hybridization sequence and detecting a series of fluorescent signals associated with the first sequence to determine the first nucleic acid molecule and a location of the first nucleic acid molecule, followed by hybridizing a second oligonucleotide to the second oligonucleotide binding sequence, and detecting a series of fluorescent signals associated with the second sequence to determine the second nucleic acid molecule and a location of the second nucleic acid molecule, wherein the first oligonucleotide and the second oligonucleotide include different sequences; and iv) generating an image of the tissue sample including indicia data corresponding to the locations of the first and second nucleic acid molecules.

In embodiments, the method includes identifying the patient as having inflammatory bowel disease (IBD) when the generated image of the tissue includes a spatial pattern of gene expression associated with IBD. As used herein, a “spatial pattern” refers to the arrangement and distribution of detectable indicia (e.g., fluorescent spots, dots, or features) corresponding to nucleic acid molecules that have been identified and localized within the tissue section. The spatial pattern therefore reflects not only the identity of the transcript or protein detected but also its position relative to other transcripts, cell boundaries, tissue structures, and histological features. The indicia may be overlaid on a histological image of the tissue, such as a fluorescent H&E (fH&E) image, to produce a composite representation in which each gene or protein target is visualized as a dot or feature at its physical location in the sample. For example, dots representing TNF transcripts may appear clustered within inflamed crypt regions of colonic mucosa, whereas IFNG transcripts may be visualized within granulomatous lesions characteristic of Crohn's disease. Similarly, MUC2 expression may be observed along epithelial cell layers, with loss or reduction of signal in ulcerated regions, providing a spatially resolved molecular correlate of tissue pathology. Clusters of IL1B and IL6 transcripts within the lamina propria, or co-localization of FOXP3 and IL10 transcripts within regulatory T cells, may constitute spatial signatures of ulcerative colitis. In other embodiments, the spatial pattern is characterized by distribution relative to tissue landmarks, such as the presence of pro-inflammatory transcripts at the base of crypts, around ulcerated mucosal surfaces, or within mesenteric lymphoid aggregates. In embodiments, the spatial pattern includes ratios or relative distributions of pro- versus anti-inflammatory signals. For example, an increased ratio of TNF and IFNG transcripts to IL10 transcripts, reflected as relative densities of corresponding dots in the composite image, may indicate an active IBD lesion. In further embodiments, the spatial pattern comprises the neighbor relationships between transcripts and proteins, such as IL17A transcripts detected in CD4+ T cells positioned adjacent to epithelial cells, potentially suggesting barrier disruption. Accordingly, the spatial pattern of indicia overlaid on the tissue image provides a molecularly resolved visualization of IBD pathology. The detection of such patterns enables differentiation between ulcerative colitis and Crohn's disease, assessment of disease severity, and prediction of therapeutic response, based on composite images that integrate both morphology and spatially resolved gene expression.

In embodiments, the spatial pattern includes clustering (e.g., computationally grouping) of TNF, IL1B, and IL6 transcripts within colonic mucosa. In embodiments, the spatial pattern includes identifying granulomatous localization of IFNG, CXCL9, and CD68 transcripts within the tissue sample. In embodiments, the spatial pattern includes detection of IL17A transcripts within CD4+ T cells located at crypt borders of the tissue. In embodiments, the method includes identifying the patient as having inflammatory bowel disease (IBD) when the generated image of the tissue sample includes an elevated abundance of IBD-associated genes (e.g., relative to a control).

In an aspect is provided a method of detecting a nucleic acid molecule in a tissue, the method including: immobilizing a tissue to a solid support, wherein the tissue includes a nucleic acid molecule and is selected from colon tissue, rectal tissue, ileal tissue, small intestine tissue, gastric tissue, esophageal tissue, perianal tissue, fibrotic tissue, and granulomatous tissue; contacting the tissue sample with an oligonucleotide probe, wherein the oligonucleotide probe includes a first hybridization sequence and a second hybridization sequence; hybridizing the first hybridization sequence to the nucleic acid molecule and hybridizing the second hybridization sequence to the nucleic acid molecule; ligating the first hybridization sequence to the second hybridization sequence to form a circular polynucleotide; amplifying the circular polynucleotide to form an amplification product; and detecting (e.g., sequencing) a sequence of the amplification product. In embodiments, detecting includes sequencing.

In embodiments, prior to sequencing, the method includes amplifying the oligonucleotide probe. For example, following annealing the oligonucleotide probe to the nucleic acid molecule, the first sequence and the second sequence are ligated together to form a circular polynucleotide. The circular polynucleotide may then be amplified using rolling circle amplification (RCA) to form amplification produces including copies of the first sequence and the second sequence (and/or complements thereof) which may then be sequenced. In embodiments, amplification is performed at a temperature between or between about 20° C. and about 60° C. In embodiments, amplification is performed at a temperature between or between about 30° C. and about 40° C. In some embodiments, the amplification step, such as the rolling circle amplification (RCA) is performed at a temperature between at or about 25° C. and at or about 50° C., e.g., 25° C., 27° C., 29° C., 31° C., 33° C., 35° C., 37° C., 39° C., 41° C., 43° C., 45° C., 47° C., or 49° C. In embodiments, sequencing the sequence includes sequencing the first hybridization sequence, the second hybridization sequence, or a barcode sequence. In embodiments, the method includes detecting the nucleic acid molecule by identifying the first hybridization sequence, the second hybridization sequence, or both the first and second hybridization sequences. In embodiments, the method includes detecting the nucleic acid molecule by identifying the first hybridization sequence. In embodiments, the method includes detecting the nucleic acid molecule by identifying the second hybridization sequence. In embodiments, the method includes detecting the nucleic acid molecule by identifying both the first and second hybridization sequences.

In embodiments the probe oligonucleotides include a sequence provided herein, or a sequence with 80% or more homology with a sequence described herein. In embodiments, the probe oligonucleotide includes a sequence that is 85%, 90%, 95%, 96%, 97%, 98%, 99%, or 100% identical to a sequence described herein. In embodiments, the oligonucleotide probe further includes a barcode sequence. In embodiments, the oligonucleotide probe includes DNA. In embodiments, the oligonucleotide probe consists of DNA. In embodiments, the oligonucleotide probe is a single-stranded polynucleotide comprising at least one amplification primer binding sequence, at least one sequencing primer binding sequence, or both one amplification primer binding sequence and one sequencing primer binding sequence. In embodiments, the first hybridization sequence, the second hybridization sequence, or both the first and second hybridization sequences of the polynucleotide probe comprise one or more locked nucleic acid (LNA) nucleotides. In embodiments, an amplification primer and a sequencing primer are complementary to different primer binding sequences. In embodiments, the primer binding sequence is complementary to a fluorescent in situ hybridization (FISH) probe. FISH probes may be custom designed using known techniques in the art, see for example Gelali, E., Girelli, G., Matsumoto, M. et al. Nat Commun 10, 1636 (2019).

In embodiments, the methods of detection are performed in multiplex assays, whereby a plurality of target molecules are detected in the same assay (a single reaction mixture). In embodiments, the plurality of target molecules is detected simultaneously. In embodiments, the plurality of target molecules detected in the same assay is at least 5 different target molecules, at least 10 different target molecules, at least 20 different target molecules, at least 50 different target molecules, at least 75 different target molecules, at least 100 different target molecules, at least 200 different target molecules, at least 500 different target molecules, or at least 750 different target molecules, or at least 1000 different target molecules. In embodiments, the plurality of target molecules detected in the same assay is up to 50 different target molecules, up to 100 different target molecules, up to 150 different target molecules, up to 200 different target molecules, up to 300 different target molecules, up to 500 different target molecules, up to 750 different target molecules, up to 1000 different target molecules, up to 2000 different target molecules, or up to 5000 different target molecules. In embodiments, the plurality of target molecules detected is any range in between the foregoing numbers of different target molecules, such as, but not limited to, from 20 to 50 different target molecules, from 50 to 200 different target molecules, from 100 to 1000 different target molecules, or from 500 to 5000 different target molecules.

In embodiments, the method further includes detecting a target protein molecule. In embodiments, the method includes detecting the target protein molecule in a cell or tissue. In embodiments, the method includes binding a polynucleotide probe to an oligonucleotide, wherein the oligonucleotide is covalently bound to a specific binding agent (e.g., an antibody); detecting the polynucleotide probe, thereby detecting the target molecule. In embodiments, detecting includes serially contacting the oligonucleotides with labeled probes (e.g., labeled oligonucleotides or labeled nucleotides).

In embodiments, the polynucleotide probe includes a fluorescently labeled probe. The phrase “labeled probes” refers to mixture of nucleic acids that are detectably labeled, e.g., fluorescently labeled, such that the presence of the probe, as well as, any target sequence to which the probe is bound can be detected by assessing the presence of the label. In embodiments, the probes are about 30-300 bases in length, 40-300 bases in length, or 70-300 bases in length. In some embodiments, the probes are relatively uniform in length (e.g., an average length+/−10 bases). The probes may be uniformly labeled based on position of label and/or number of labels within the probe. In some embodiments, the probes are single-stranded. In some embodiments, the probes are double-stranded. Additional detection probes and related properties may be found in, e.g., U.S. Pat. Pub. US 2011/0039735, which is incorporated herein by reference in its entirety. In embodiments, the method includes binding a polynucleotide probe including a first binding sequence and a second binding sequence to an oligonucleotide, wherein the oligonucleotide is attached to the target molecule (e.g., covalently attached to an antibody, wherein the antibody is specifically bound to the target protein molecule); amplifying the polynucleotide probe to form an amplification product comprising one or more copies of the first binding sequence and the second binding sequence; hybridizing a primer to the amplification product and incorporating a labeled nucleotide into the primer, thereby detecting the target protein molecule. In embodiments, the method includes hybridizing a labeled probe to the amplification product and detecting the labeled probe, thereby detecting the target protein molecule.

In embodiments, the method includes detecting biomolecules in a tissue sample. The biological targets or molecules to be detected can be any biological molecules including but not limited to proteins, nucleic acids, lipids, carbohydrates, ions, or multicomponent complexes containing any of the above. Examples of subcellular targets include organelles, e.g., mitochondria, Golgi apparatus, endoplasmic reticulum, chloroplasts, endocytic vesicles, exocytic vesicles, vacuoles, lysosomes, etc. Exemplary nucleic acid targets can include genomic DNA of various conformations (e.g., A-DNA, B-DNA, Z-DNA), mitochondria DNA (mtDNA), mRNA, tRNA, rRNA, hRNA, miRNA, and piRNA. For example, following immobilization on the receiving substrate, the sections may be fixed with methanol, permeabilized with 0.025% Triton in PBS solution, and stained with primary antibodies directed against vimentin (fibroblasts) and macrophages, followed by secondary antibody labeling (e.g., Alexa-594 conjugated secondary antibodies). Additional counterstaining may be performed, for example using 4,6-diamidino-2-phenylindole (DAPI) mounting media to counterstain nuclei. In embodiments, the tissue sample includes colon tissue, rectal tissue, ileal tissue, small intestine tissue, Peyer's patches, mesenteric lymph nodes, gastric tissue, esophageal tissue, perianal tissue, fibrotic tissue, granulomatous tissue, and/or fistula tissue.

In embodiments, the tissue is a tissue section. In embodiments, the tissue section includes a tissue or a cell (e.g., plurality of cells such as blood cells). In embodiments, the tissue section includes one or more cells. In embodiments, the thickness of the tissue section is about 1 μm to about 20 μm. In embodiments, the thickness of the tissue section is about 5 μm to about 12 μm. In embodiments, the thickness of the tissue section is about 8 μm to about 15 μm. In embodiments, the thickness of the tissue section is about 1 μm, about 2 μm, about 3 μm, about 4 μm, about 5 μm, about 6 μm, about 7 μm, about 8 μm, about 9 μm, about 10 μm, about 11 μm, about 12 μm, about 13 μm, about 14 μm, or about 15 μm. In embodiments, the thickness of the tissue section is about 1 μm. In embodiments, the thickness of the tissue section is about 2 μm. In embodiments, the thickness of the tissue section is about 3 μm. In embodiments, the thickness of the tissue section is about 4 μm. In embodiments, the thickness of the tissue section is about 5 μm. In embodiments, the thickness of the tissue section is about 6 μm. In embodiments, the thickness of the tissue section is about 7 μm. In embodiments, the thickness of the tissue section is about 8 μm. In embodiments, the thickness of the tissue section is about 9 μm. In embodiments, the thickness of the tissue section is about 10 μm. In embodiments, the thickness of the tissue section is about 11 μm. In embodiments, the thickness of the tissue section is about 12 μm. In embodiments, the thickness of the tissue section is about 13 μm. In embodiments, the thickness of the tissue section is about 14 μm. In embodiments, the thickness of the tissue section is about 15 μm. In embodiments, the thickness of the tissue section is less than about 10 μm. In embodiments, the thickness of the tissue section is less about 6 μm, 7 μm, 8 μm, 9 μm or 10 μm. In embodiments, the cell or tissue is obtained via Laser capture microdissection (LCM). LCM is a method for isolating in micrometer-scale tissue or even single cells while retaining spatial information to link histology with molecular measurements. In LCM, a region of interest in the tissue section is isolated through laser cutting.

Tissue sections, e.g., tumor tissue samples, may be obtained surgically or using a laparoscope. A tissue section may be a tissue sample obtained from any part of the body to examine it for disease or injury, e.g., presence of cancer tissue or cells, or the extent or characteristics thereof. In particular embodiments, the tissue section includes abdominal tissue, bone, bone marrow, breast tissue, endometrial tissue, kidney tissue, liver tissue, lung or chest tissue, lymph node, nerve tissue, skin, testicular tissue, head or neck tissue, or thyroid tissue. In certain embodiments, the tissue is obtained from brain, breast, skin, bone, joint, skeletal muscle, smooth muscle, red bone marrow, thymus, lymphatic vessel, thoracic duct, spleen, lymph node, nasal cavity, pharynx, larynx, trachea, bronchus, lung, oral cavity, esophagus, liver, stomach, small intestine, large intestine, rectum, anus, spinal cord, nerve, pineal gland, pituitary gland, thyroid gland, thymus, adrenal gland, pancreas, ovary, testis, heart, blood vessel, kidney, uterus, urinary bladder, urethra, prostate gland, penis, prostate, testis, scrotum, ductus deferens, mammary glands, ovary, uterus, vagina, or uterine tube.

In embodiments, the tissue section includes a tissue or a cell. Biological tissue samples suitable for use with the methods and systems described herein generally include any type of tissue samples collected from living or dead subjects, such as, for example, tumor tissue and autopsy samples. Tissue samples may be collected and processed using the methods and systems described herein and subjected to microscopic analysis immediately following processing, or may be preserved and subjected to microscopic analysis at a future time, e.g., after storage for an extended period of time. In some embodiments, the methods described herein may be used to preserve tissue samples in a stable, accessible and fully intact form for future analysis. For example, tissue samples, such as, e.g., human tumor tissue samples, may be processed as described herein and cleared to remove a plurality of cellular components, such as, e.g., lipids, and then stored for future analysis. In some embodiments, the methods and systems described herein may be used to analyze a fresh tissue section. In some embodiments, the methods and systems described herein may be used to analyze a previously-preserved (e.g., previously fixed) or stored tissue section (e.g., tissue sample). For example, in some embodiments a previously-preserved tissue sample that has not been subjected to a sample preparation process described herein may be processed and analyzed as described herein. In particular methods, a tissue sample is frozen prior to being processed as described herein.

In embodiments, prior to contacting the tissue with a probe oligonucleotide, the method further includes immobilizing the tissue section onto a solid support (e.g., a flow cell or well in a microplate). In embodiments, the solid support includes a glass substrate. In embodiments, the glass substrate is a borosilicate glass substrate with a composition including SiO₂, Al₂O₃, B₂O₃, Li₂O, Na₂O, K₂O, MgO, CaO, SrO, BaO, ZnO, TiO₂, ZrO₂, P₂O₅, or a combination thereof (see e.g., U.S. Pat. No. 10,974,990). In embodiments, the glass substrate is an alkaline earth boro-aluminosilicate glass substrate.

In embodiments, the solid support includes a polymer layer. In embodiments, the polymer layer includes polymerized units of alkoxysilyl methacrylate, alkoxysilyl acrylate, alkoxysilyl methylacrylamide, alkoxysilyl methylacrylamide, or a copolymer thereof. In embodiments, the polymer layer includes polymerized units of alkoxysilyl methacrylate. In embodiments, the polymer layer includes polymerized units of alkoxysilyl acrylate. In embodiments, the polymer layer includes polymerized units of alkoxysilyl methylacrylamide. In embodiments, the polymer layer includes polymerized units of alkoxysilyl methylacrylamide. In embodiments, the polymer layer includes glycidyloxypropyl-trimethyloxysilane. In embodiments, the polymer layer includes methacryloxypropyl-trimethoxysilane. In embodiments, the polymer layer includes polymerized units of

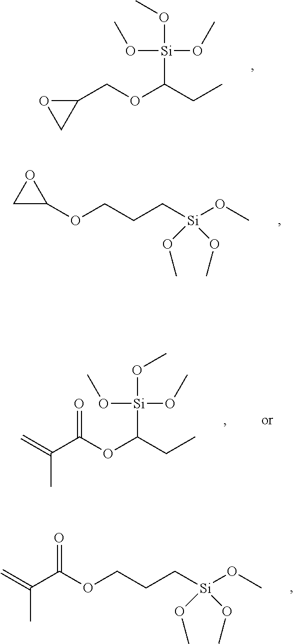

or a copolymer thereof. In embodiments, the polymer layer is an organically-modified ceramic polymer. In embodiments, the polymer includes polymerized monomers of alkoxysilyl polymers, such as

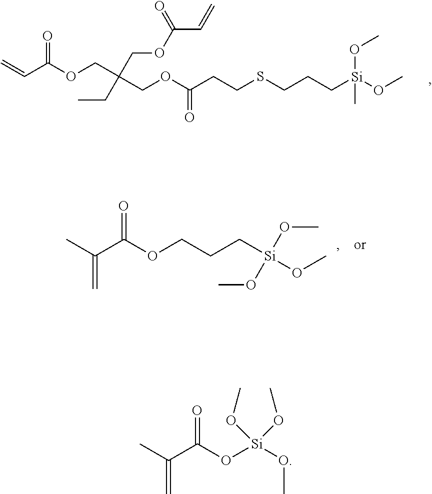

In embodiments, the solid support includes polymerized units of

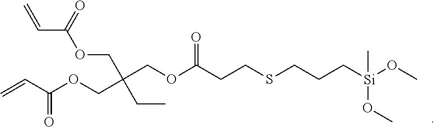 [SMILES: C=CC(=O)OCC(CC)(COC(=O)C=C)COC(=O)CCSCCC[Si](C)(OC)OC]

In embodiments, the solid support includes polymerized units of

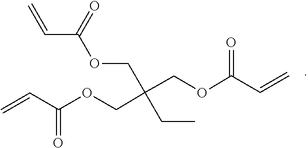 [SMILES: C=CC(=O)OCC(CC)(COC(=O)C=C)COC(=O)C=C]

In embodiments, the solid support includes polymerized unites of

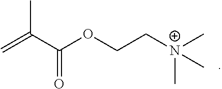

In embodiments, the polymer layer includes one or more ceramic particles, (e.g., silicates, aluminates, and titanates). In embodiments, the polymer layer includes tantalum(V) oxide, titanium dioxide, zinc oxide, and/or iron oxide.

In embodiments, the method further includes permeabilizing the tissue prior to binding the probe to the biomolecule. Methods for permeabilization are known in the art, as exemplified by Cremer et al., The Nucleus: Volume 1: Nuclei and Subnuclear Components, R. Hancock (ed.) 2008; and Larsson et al., Nat. Methods (2010) 7:395-397, the content of each of which is incorporated herein by reference in its entirety. In embodiments, the tissue and/or cell is cleared (e.g., digested) of proteins, lipids, or proteins and lipids. In embodiments, permeabilizing the tissue or cell does not release the biomolecules (e.g., the one or more biomolecules) from within the tissue or cell. For example, after a fixation process (e.g. formaldehyde cross-linking), proteins and nucleic acids are immobilized within the cells of a tissue section, and are therefore not liberated into the environment following permeabilization of the cells. In embodiments, permeabilizing includes contacting the sample (e.g., the cell or tissue) with a permeabilization agent. Suitable agents for this purpose include, but are not limited to, organic solvents (e.g., acetone, ethanol, and methanol), cross-linking agents (e.g., paraformaldehyde), detergents (e.g., saponin, Triton X100™, Tween-20™, or sodium dodecyl sulfate (SDS)), and enzymes (e.g., trypsin, proteases (e.g., proteinase K). In embodiments, the detergent is an anionic detergent (e.g., SDS or N-lauroylsarcosine sodium salt solution). In embodiments, the permeabilization agent includes polyethylene glycol (PEG). In embodiments, the PEG is from about PEG 2K to about PEG 16K. In some embodiments, the PEG is PEG 2K, 3K, 4K, 5K, 6K, 7K, 8K, 9K, 10K, 11K, 12K, 13K, 14K, or 16K. In embodiments, the PEG is present at a concentration from about 2% to 25%, from about 4% to about 23%, from about 6% to about 21%, or from about 8% to about 20% (v/v).

Imaging deep into a tissue volume is problematic due to inherently fluorescent molecules present in the tissue or introduced during processing which give rise to autofluorescence that masks fluorescently labelled structures of interest. Typically, autofluorescence decreases image quality by lowering the signal to noise ratio across multiple fluorescence channels and undermines sharp images. Autofluorescence may arise from endogenous fluorescent biomolecules (NADPH, collagen, flavins, tyrosine, and others) or be introduced by the formation of Schiffs bases during fixation with aldehydes (e.g., glutaraldehyde and paraformaldehyde). Additional light scattering is provided by various cellular components, such as ribosomes, nuclei, nucleoli, mitochondria, lipid droplets, membranes, myelin, cytoskeletal components, and extracellular matrix components such as collagen and elastin.

In embodiments, the tissue is cleared using a solvent-based clearing approach. Solvent-based clearing techniques typically includes two steps: 1) dehydration (e.g., contacting the sample with methanol with or without hexane or, tetrahydrofuran (THF) alone) and 2) clearing by refractive index matching to the remaining dehydrated tissue's index (e.g., contacting the tissue sample with methyl salicylate, benzyl alcohol, benzyl benzoate, dichloromethane, or dibenzyl ether). Alternatively, the initial dehydration may be performed using phosphate buffered saline (PBS), detergent, and dimethyl sulfoxide (DMSO). In embodiments, the tissue is cleared by contacting the tissue sample with an aqueous solution containing sucrose, fructose, 2,2′-thiodiethanol (TDE), or formamide.

In embodiments, the tissue is cleared utilizing the 3D imaging of solvent-cleared organs (3DISCO) method as described in Ertürk A et al. Nat Protoc. 2012 November; 7(11):1983-95, which is incorporated herein by reference. For example, a sample is incubated overnight in 50% v/v tetrahydrofuran/H₂O (THF), followed by incubation for at least one hour 80% THF/H₂O and followed by incubation in a 100% THF solution. This is then followed by contacting the sample with dichloromethane (DCM) and an incubation in dibenzyl ether (DBE) until clear. In embodiments, the tissue is cleared according to a known technique in the art, for example CLARITY (Chung K., et al. Nature 497, 332-337 (2013)), PACT-PARS (Yang B et al. Cell 158, 945-958 (2014)), CUBIC (Susaki E. A. et al. Cell 157, 726-739 (2014), 18), ScaleS (Hama H., et al. Nat. Neurosci. 18, 1518-1529 (2015)), OPTIClear (Lai H. M., et al. Nat. Commun. 9, 1066 (2018)), Cₑ3D (Li W., et al. Proc. Natl. Acad. Sci. U.S.A. 114, E7321-E7330 (2017)), BABB (Dodt H. U. et al. Nat. Methods 4, 331-336 (2007)), iDISCO (Renier N., et al. Cell 159, 896-910 (2014)), uDISCO (Pan C., et al. Nat. Methods 13, 859-867 (2016)), FluoClearBABB (Schwarz M. K., et al. PLOS ONE 10, e0124650 (2015)), Ethanol-ECi (Klingberg A., et al. J. Am. Soc. Nephrol. 28, 452-459 (2017)), and PEGASOS (Jing D. et al. Cell Res. 28, 803-818 (2018)).

In embodiments, the tissue is contacted with an alkaline solution containing a combination of 2,2′-thiodiethanol (TDE), DMSO, D-sorbitol, and Tris. In embodiments, the tissue section is contacted with an aqueous solution including 20% (vol/vol) DMSO, 40% (vol/vol) TDE, 20% (wt/vol) sorbitol, and 6% (wt/vol, equal to 0.5 M) Tris base. In embodiments, the tissue section is contacted with an aqueous solution including 25% (wt/wt) urea, 25% (wt/wt) N,N,N′,N′-Tetrakis (2-hydroxypropyl) ethylenediamine, and 15% (wt/wt) Triton X-100. In embodiments, the tissue section is contact with an aqueous solution including 9.1 M urea, 22.5% (wt/vol) D-sorbitol, and 5% (wt/vol) Triton X-100. In embodiments, the tissue section is contact with an aqueous solution including 30% (wt/vol) urea, 20% (wt/vol) D-sorbitol, and 5% (wt/vol) glycerol dissolved in DMSO. In embodiments, the tissue or cell is contacted with an aqueous solution according to the protocols described in Shan, Q H., Qin, X Y., Zhou, N. et al. BMC Biol 20, 77 (2022).

To microscopically visualize tissue sections prepared by the subject methods, in some embodiments the tissue section is embedded in a mounting medium. Mounting medium is typically selected based on its suitability for the reagents used to visualize the cellular biomolecules, the refractive index of the tissue section, and the microscopic analysis to be performed. For example, for phase-contrast work, the refractive index of the mounting medium should be different from the refractive index of the specimen, whereas for bright-field work the refractive indexes should be similar. As another example, for epifluorescence work, a mounting medium should be selected that reduces fading, photobleaching or quenching during microscopy or storage. In certain embodiments, a mounting medium or mounting solution may be selected to enhance or increase the optical clarity of the cleared tissue specimen. Nonlimiting examples of suitable mounting media that may be used include glycerol, CC/Mount™, Fluoromount™ Fluoroshield™, ImmunHistoMount™, Vectashield™, Permount™, Acrytol™, CureMount™ FocusClear™, or equivalents thereof.

In an aspect is provided a method of imaging a cell or tissue. In embodiments, the method includes: contacting a cell or tissue with a first probe set, wherein the first probe set includes a plurality of probes as described herein; binding each probe to a different target of the cell or tissue; imaging (e.g., obtaining an image) the cell or tissue; contacting the cell or tissue with a second probe set, wherein the second probe set includes a plurality of probes as described herein; binding each probe to a different target of the cell or tissue; and imaging (e.g., obtaining an image) the cell or tissue.

In embodiments, amplifying the circular polynucleotide generates an amplification product including multiple copies of the target sequence, or a complement thereof. In embodiments, the method includes serially cycling through detection cycles to determine the sequence (e.g., the order of the nucleotides) of the target sequence), wherein each detection cycle includes hybridizing, detecting, and removing a fluorescently labelled oligonucleotide. In embodiments, detecting includes sequencing the amplification product (e.g., using a sequencing by synthesis or sequencing by binding process).

In embodiments, forming the circular polynucleotide includes ligating a first end and a second end of the oligonucleotide probe together. In embodiments, ligating includes forming a covalent bond from the first end and the second end. As those of skill in the art appreciate, two nucleotide sequences that that are to ligated together will generally directly abut one another.

In embodiments, the method includes contacting a cell or tissue with a polynucleotide probe and hybridizing a first hybridization sequence of the polynucleotide probe to a first target sequence of the RNA or DNA molecule, and hybridizing a second hybridization sequence of the polynucleotide probe to a second target sequence of the RNA or DNA molecule, ligating the first hybridization sequence and the second hybridization sequence together thereby forming a circular oligonucleotide; extending an amplification primer hybridized to the circular oligonucleotide with a polymerase to generate an extension product comprising the target sequence; and hybridizing a sequencing primer to the extension product and sequencing the target sequence (i.e., the first and/or second hybridization sequence) in the cell or tissue, thereby detecting the RNA or DNA molecule. In embodiments, the nucleic acid molecule is cDNA. In embodiments, the nucleic acid molecule is DNA. In embodiments, the nucleic acid molecule is RNA. In embodiments, the target is an RNA nucleic acid sequence or DNA nucleic acid sequence. In embodiments, the target is an RNA nucleic acid sequence or DNA nucleic acid sequence from the same cell.

In embodiments, the method includes circularizing and ligating the complementary sequence to the 5′ end of the polynucleotide probe. In embodiments, circularizing the oligonucleotide primer to generate a circular oligonucleotide includes extending the 3′ end of the oligonucleotide primer (e.g., extending the 3′ end of the primer using a polymerase (e.g., a Thermus thermophilus (Tth) DNA polymerase) to incorporate one or more nucleotides) along the target nucleic acid to generate a complementary sequence (e.g., complementary to the target nucleic acid, for example a target RNA sequence), and ligating the complementary sequence to the 5′ end of the oligonucleotide primer. In embodiments, the ligation includes enzymatic ligation. In embodiments, ligating includes enzymatic ligation including a ligation enzyme (e.g., Circligase enzyme, Taq DNA Ligase, HiFi Taq DNA Ligase, T4 ligase, PBCV-1 DNA Ligase (also known as SplintR® ligase) or Ampligase DNA Ligase). Non-limiting examples of ligases include DNA ligases such as DNA Ligase I, DNA Ligase II, DNA Ligase III, DNA Ligase IV, T4 DNA ligase, T7 DNA ligase, T3 DNA Ligase, E. coli DNA Ligase, PBCV-1 DNA Ligase (also known as SplintR ligase) or a Taq DNA Ligase. In embodiments, the ligase enzyme includes a T4 DNA ligase, T4 RNA ligase 1, T4 RNA ligase 2, T3 DNA ligase or T7 DNA ligase. In embodiments, the enzymatic ligation is performed by a mixture of ligases. In embodiments, the ligation enzyme is selected from the group consisting of T4 DNA ligase, T4 RNA ligase 1, T4 RNA ligase 2, RtcB ligase, T3 DNA ligase, T7 DNA ligase, Taq DNA ligase, PBCV-1 DNA Ligase, a thermostable DNA ligase (e.g., 5′AppDNA/RNA ligase), an ATP dependent DNA ligase, an RNA-dependent DNA ligase (e.g., SplintR ligase), and combinations thereof.

In embodiments, amplifying the circular polynucleotide includes hybridizing a primer to the circular polynucleotide and extending the primer with a strand-displacing polymerase. In embodiments, the method further includes amplifying the circular oligonucleotide by extending an amplification primer with a polymerase (e.g., a strand-displacing polymerase), wherein the primer extension generates an extension product including multiple complements of the circular oligonucleotide, referred to as an amplicon. An amplicon typically contains multiple, tandem copies of the circularized nucleic acid molecule of the corresponding sample nucleic acid. The number of copies can be varied by appropriate modification of the reaction conditions, such as varying the number of amplification cycles, using polymerases of varying processivity in the amplification reaction, or varying the length of time that the amplification reaction is run. In embodiments, the extension product includes three or more copies of the circular oligonucleotide. In embodiments, the circular oligonucleotide is copied about 3-50 times (i.e., the extension product includes about 3 to 50 complements of the circular oligonucleotide). In embodiments, the circular oligonucleotide is copied about 50-100 times (i.e., the extension product includes about 50 to 100 complements of the circular oligonucleotide). In embodiments, the circular oligonucleotide is copied about 100-300 times (i.e., the extension product includes about 100 to 300 complements of the circular oligonucleotide). In embodiments, the method includes hybridizing an amplification primer and oligonucleotide primer in the same reaction (e.g., simultaneously). In embodiments, the oligonucleotide is extended as an amplification primer after generating the circular oligonucleotide (e.g., the 3′ end of the oligonucleotide hybridized to the circular oligonucleotide is extended with a polymerase). In embodiments, the method includes contacting the target with an amplification primer and oligonucleotide primer in the same reaction (e.g., simultaneously). In embodiments, the method includes fixing the amplification products (e.g., contacting the amplification product with formalin). In embodiments, amplifying includes incubating the circular polynucleotide with a strand-displacing polymerase for about 15 minutes to about 2 hours. In embodiments, amplifying includes incubating the circular polynucleotide with a strand-displacing polymerase for about 30 minutes to about 60 minutes. In embodiments, amplifying includes binding an amplification primer to the primer binding sequence and extending the amplification primer with a strand-displacing polymerase.

In embodiments, sequencing includes hybridizing a sequencing primer to the amplification product and incorporating one or more labeled nucleotides, and detecting the incorporated one or more labeled nucleotides so as to identify the sequence.

In embodiments, the method includes sequencing the extension products, which includes the target nucleic acid sequence. A variety of sequencing methodologies can be used such as sequencing-by synthesis (SBS), pyrosequencing, sequencing by ligation (SBL), or sequencing by hybridization (SBH). Pyrosequencing detects the release of inorganic pyrophosphate (PPi) as particular nucleotides are incorporated into a nascent nucleic acid strand (Ronaghi, et al., Analytical Biochemistry 242(1), 84-9 (1996); Ronaghi, Genome Res. 11(1), 3-11 (2001); Ronaghi et al. Science 281(5375), 363 (1998); U.S. Pat. Nos. 6,210,891; 6,258,568; and. 6,274,320, each of which is incorporated herein by reference in its entirety). In pyrosequencing, released PPi can be detected by being converted to adenosine triphosphate (ATP) by ATP sulfurylase, and the level of ATP generated can be detected via light produced by luciferase. In this manner, the sequencing reaction can be monitored via a luminescence detection system. In both SBL and SBH methods, target nucleic acids, and amplicons thereof, are subjected to repeated cycles of oligonucleotide delivery and detection. SBL methods, include those described in Shendure et al. Science 309:1728-1732 (2005); U.S. Pat. Nos. 5,599,675; and 5,750,341, each of which is incorporated herein by reference in its entirety; and the SBH methodologies are as described in Bains et al., Journal of Theoretical Biology 135(3), 303-7 (1988); Drmanac et al., Nature Biotechnology 16, 54-58 (1998); Fodor et al., Science 251(4995), 767-773 (1995); and WO 1989/10977, each of which is incorporated herein by reference in its entirety.

In SBS, extension of a nucleic acid primer along a nucleic acid template is monitored to determine the sequence of nucleotides in the template. The underlying chemical process can be catalyzed by a polymerase, wherein fluorescently labeled nucleotides are added to a primer (thereby extending the primer) in a template dependent fashion such that detection of the order and type of nucleotides added to the primer can be used to determine the sequence of the template. In embodiments, sequencing includes annealing and extending a sequencing primer to incorporate a detectable label that indicates the identity of a nucleotide in the target polynucleotide, detecting the detectable label, and repeating the extending and detecting of steps. In embodiments, the methods include sequencing one or more bases of a target nucleic acid by extending a sequencing primer hybridized to a target nucleic acid (e.g., an amplification product produced by the amplification methods described herein). In embodiments, sequencing may be accomplished by a sequencing-by-synthesis (SBS) process. In embodiments, sequencing includes a sequencing by synthesis process, where individual nucleotides are identified iteratively, as they are polymerized to form a growing complementary strand. In embodiments, nucleotides added to a growing complementary strand include both a label and a reversible chain terminator that prevents further extension, such that the nucleotide may be identified by the label before removing the terminator to add and identify a further nucleotide. Such reversible chain terminators include removable 3′ blocking groups, for example as described in U.S. Pat. Nos. 7,541,444 and 7,057,026. Once such a modified nucleotide has been incorporated into the growing polynucleotide chain complementary to the region of the template being sequenced, there is no free 3′-OH group available to direct further sequence extension and therefore the polymerase cannot add further nucleotides. Once the identity of the base incorporated into the growing chain has been determined, the 3′ reversible terminator may be removed to allow addition of the next successive nucleotide. By ordering the products derived using these modified nucleotides it is possible to deduce the DNA sequence of the oligonucleotide target nucleic acid sequence.

In embodiments, sequencing includes sequentially extending a plurality of sequencing primers (e.g., sequencing a first region of a target nucleic acid followed by sequencing a second region of a target nucleic acid, followed by sequencing N regions, where N is the number of sequencing primers in the known sequencing primer set). In embodiments, sequencing includes generating a plurality of sequencing reads.

In embodiments, the method includes sequencing the amplification products (e.g., a plurality of different amplification products). In embodiments, sequencing includes a plurality of sequencing cycles. In embodiments, sequencing includes a plurality of rounds of sequencing cycles (e.g., a first round of 10 sequencing cycles; followed by a second round of 10 sequencing cycles). In embodiments, sequencing includes a plurality of rounds of sequencing cycles (e.g., a first round of 1 sequencing cycle; followed by a second round of 1 sequencing cycle). In embodiments, sequencing includes 20 to 100 sequencing cycles. In embodiments, sequencing includes 50 to 100 sequencing cycles. In embodiments, sequencing includes 50 to 300 sequencing cycles. In embodiments, sequencing includes 50 to 150 sequencing cycles. In embodiments, sequencing includes at least 10, 20, 30 40, or 50 sequencing cycles. In embodiments, sequencing includes at least 10 sequencing cycles. In embodiments, sequencing includes 10 to 20 sequencing cycles. In embodiments, sequencing includes 10, 11, 12, 13, 14, or 15 sequencing cycles. In embodiments, sequencing includes (a) extending a sequencing primer by incorporating a labeled nucleotide, or labeled nucleotide analogue and (b) detecting the label to generate a signal for each incorporated nucleotide or nucleotide analogue. In embodiments, prior to initiating a next round of sequencing cycles, the first sequencing primer is terminated or removed. For example, termination may occur via incorporating a non-extendable nucleotide (e.g., a ddNTP or acyclic nucleotide) into the first sequencing primer.

In embodiments, sequencing includes sequentially sequencing a plurality of different targets by initiating sequencing with different sequencing primers. For example, a first circularizable probe includes a first primer binding site (a nucleic acid sequence complementary to a first sequencing primer) and optionally a first barcode sequence or barcode nucleotide. In a similar manner, a second and third padlock probe include a second primer binding site (a nucleic acid sequence complementary to a second, different, sequencing primer) and a third primer binding site (a nucleic acid sequence complementary to a third, different from both Primer 1 and Primer 2, sequencing primer), respectively. During the first round of sequencing (following probe circularization and amplification according to the methods described herein), using primer 1, the probe hybridized to the first nucleic acid molecule is detected. In the second round of sequencing, primer 2 can hybridize and sequence an identifying sequence of the probe (e.g., a barcode sequence or nucleotide) hybridized to a second nucleic acid molecule. Similarly, in the third round of sequencing, primer 3 can hybridize and sequence the probe hybridized to the third nucleic acid molecule. In embodiments, each oligonucleotide probe further includes a barcode sequence. In embodiments, the barcode (i.e., the barcode sequence) is at least 5, 6, 7, 8, 9, 10, 11, 12, 13, 14, or 15 nucleotides in length. In embodiments, the barcode is 5, 6, 7, 8, 9, 10, 11, 12, 13, 14, or 15 nucleotides in length. In embodiments, the barcode is 10 to 15 nucleotides in length. In embodiments, the barcode is at least about 1, 2, 3, 4, 5, 6, 7, 8, 9, 10, 15, 20, 25, 30, 35, 40, 45, 50 or more nucleotides in length. In embodiments, the barcode can be at most about 300, 200, 100, 90, 80, 70, 60, 50, 40, 30, 20, 15, 12, 10, 9, 8, 7, 6, 5, 4 or fewer or more nucleotides in length. In embodiments, the barcode includes between about 5 to about 8, about 5 to about 10, about 5 to about 15, about 5 to about 20, about 10 to about 150 nucleotides. In embodiments, the barcode includes between 5 to 8, 5 to 10, 5 to 15, 5 to 20, 10 to 150 nucleotides. In embodiments, the barcode is 10 nucleotides. In embodiments, the barcode may include a unique sequence (e.g., a barcode sequence) that gives the barcode its identifying functionality. The unique sequence may be random or non-random. The random sequence can be of any suitable length, and there may be one or more than one present. As non-limiting examples, the random sequence may have a length of 10 to 40, 10 to 30, 10 to 20, 25 to 50, 15 to 40, 15 to 30, 20 to 50, 20 to 40, or 20 to 30 nucleotides.

In embodiments, sequencing includes encoding the sequencing read into a codeword. Useful encoding schemes include those developed for telecommunications, coding theory and information theory such as those set forth in Hamming, Coding and Information Theory, 2^{nd} Ed. Prentice Hall, Englewood Cliffs, N.J. (1986) and Moon T K. Error Correction Coding: Mathematical Methods and Algorithms. ed. 1st Wiley: 2005, each of which are incorporated herein by reference. A useful encoding scheme uses a Hamming code. A Hamming code can provide for signal (and therefore sequencing and barcode) distinction. In this scheme, signal states detected from a series of nucleotide incorporation and detection events (i.e., while sequencing the oligonucleotide barcode) can be represented as a series of the digits to form a codeword, the codeword having a length equivalent to the number incorporation/detection events. The digits can be binary (e.g. having a value of 1 for presence of signal and a value of 0 for absence of the signal) or digits can have a higher radix (e.g., a ternary digit having a value of 1 for fluorescence at a first wavelength, a value of 2 for fluorescence at a second wavelength, and a value of 0 for no fluorescence at those wavelengths, etc.). Barcode discrimination capabilities are provided when codewords can be quantified via Hamming distances between two codewords (i.e., barcode 1 having codeword 1, and barcode 2 having codeword 2, etc.).

In embodiments, generating a sequencing read includes determining the identity of the nucleotides in the template polynucleotide (or complement thereof). In embodiments, a sequencing read, e.g., a first sequencing read or a second sequencing read, includes determining the identity of a portion (e.g., 1, 2, 5, 10, 20, 50 nucleotides) of the total template polynucleotide. In embodiments the first sequencing read determines the identity of 5-10 nucleotides and the second sequencing read determines the identity of more than 5-10 nucleotides (e.g., 11 to 200 nucleotides). In embodiments the first sequencing read determines the identity of more than 5-10 nucleotides (e.g., 11 to 200 nucleotides) and the second sequencing read determines the identity of 5-10 nucleotides. In embodiments, following the generation of a sequencing read, subsequent extension is performed using a plurality of standard (e.g., non-modified) dNTPs until the complementary strand is copied. In other embodiments, following the generation of a sequencing read, subsequent extension is performed using a plurality of dideoxy nucleotide triphosphates (ddNTPs) or acyclic nucleotide triphosphates to prevent further extension of the first sequencing read product during a second sequencing read. In embodiments, following the identification of at least 5-10 (e.g., 11 to 200 nucleotides, or up to 1000 nucleotides), subsequent extension is performed using a plurality of standard (e.g., non-modified) dNTPs until the complementary strand is copied. In embodiments, following the identification of at least 5-10 (e.g., 11 to 200 nucleotides, or up to 1000 nucleotides), subsequent extension is performed using a plurality of dideoxy nucleotide triphosphates (ddNTPs) or acyclic nucleotide triphosphates to prevent further extension of the sequencing read product.

In embodiments, sequencing includes sequencing by synthesis, sequencing by binding, or sequencing by ligation. In embodiments, sequencing includes extending a sequencing primer by incorporating a labeled nucleotide or labeled nucleotide analogue, and detecting the label to generate a signal for each incorporated nucleotide or nucleotide analogue, wherein the sequencing primer is hybridized to the amplification product. In embodiments, the sequencing primer includes the sequence: SEQ ID NO:10531, SEQ ID NO:10532, SEQ ID NO:10533, SEQ ID NO:10534, SEQ ID NO:10535, SEQ ID NO:10536, SEQ ID NO:10537, SEQ ID NO:10538, SEQ ID NO:10539, SEQ ID NO:10540, SEQ ID NO:10541, SEQ ID NO:10542, SEQ ID NO:10543, or SEQ ID NO:10544.

In embodiments, the method includes binding a specific binding reagent including an oligonucleotide to a protein in the tissue, binding a circularizable oligonucleotide to the oligonucleotide, circularizing the circularizable oligonucleotide to form a second circular polynucleotide, amplifying the second circular polynucleotide to form a second amplification product; and detecting the amplification product. In embodiments, the specific binding reagent is selected from an antibody, single-chain Fv fragment (scFv), antibody fragment antigen-binding (Fab), or an aptamer.

In embodiments, the method includes identifying and analyzing the unique characteristics of T-cell receptors (TCRs) in a given population of T-cells. T-cells, a type of white blood cell, play a crucial role in the immune response. Each T-cell has a unique TCR that enables it to recognize specific antigens. Clonality, in this context, pertains to the presence of T-cells with identical TCRs, indicating that they have originated from the same initial T-cell. The process of identifying clonality data typically involves sequencing, which identifies the unique genetic sequences of the TCRs. This data provides insights into the diversity and specificity of the T-cell response. In medical research and diagnostics, analyzing T-cell clonality is important for understanding immune responses in diseases such as cancer, autoimmune disorders, and infections.

In an aspect is provided a method of detecting a plurality of targets. In embodiments, the method includes sequencing a plurality of target nucleic acids of a cell in situ. In embodiments, the method includes the following steps in situ for each of the plurality of target nucleic acids: i) hybridizing an oligonucleotide primer to the target nucleic acid, wherein the oligonucleotide primer includes a first region at a 3′ end that hybridizes to a first complementary region of the target nucleic acid, and a second region at a 5′ end that hybridizes to a second complementary region of the target nucleic acid, wherein the second complementary region is 5′ with respect to the first complementary region; ii) circularizing the oligonucleotide primer to generate a circular oligonucleotide, wherein circularizing includes extending the 3′ end of the oligonucleotide primer (e.g., extending the 3′ end of the primer using a polymerase (e.g., a Thermus thermophilus (Tth) DNA polymerase) to incorporate one or more nucleotides) along the target nucleic acid to generate a complementary sequence (e.g., complementary to the target nucleic acid, for example a target RNA sequence), and ligating the complementary sequence to the 5′ end of the oligonucleotide primer; iii) amplifying the circular oligonucleotide by extending an amplification primer hybridized to the circular oligonucleotide with a strand-displacing polymerase, wherein the amplification primer extension generates an extension product including multiple complements of the circular oligonucleotide; and iv) sequencing the extension product of step (iii).

In an aspect is provided a method of detecting a plurality of different nucleic acid sequences within an optically resolved volume of a cell in situ, wherein the method includes i) associating a different oligonucleotide barcode from a known set of barcodes with each of the plurality of targets, wherein associating an oligonucleotide barcode with each of the plurality of targets includes hybridizing a padlock probe to two adjacent nucleic acid sequences of the target, wherein the padlock probe is a single-stranded polynucleotide having a 5′ and a 3′ end, and wherein the padlock probe includes a primer binding sequence from a known set of primer binding sequences; ii) sequencing each barcode to obtain a multiplexed signal in the cell in situ; iii) demultiplexing the multiplexed signal by comparison with the known set of barcodes; and iv) detecting the plurality of targets by identifying the associated barcodes detected in the cell.

In another aspect is provided a method of detecting a plurality of proteins (e.g., different proteins) within an optically resolved volume of a cell in situ, wherein the method includes i) associating a different oligonucleotide barcode from a known set of barcodes with each of the plurality of targets, wherein associating an oligonucleotide barcode with each of the plurality of targets includes contacting each of the targets with a specific binding reagent, wherein the specific binding reagent includes an oligonucleotide barcode; ii) hybridizing a padlock probe to two adjacent nucleic acid sequences of the barcode, wherein the padlock probe is a single-stranded polynucleotide having a 5′ and a 3′ end, and wherein the padlock probe includes a primer binding sequence from a known set of primer binding sequences; iii) detecting (e.g., sequencing) each barcode to obtain a multiplexed signal in the cell in situ; iv) demultiplexing the multiplexed signal by comparison with the known set of barcodes; and v) detecting the plurality of targets by identifying the associated barcodes detected in the cell. In another aspect is provided a method of detecting a plurality of proteins (e.g., different proteins) within an optically resolved volume of a cell in situ, wherein the method includes i) associating a different oligonucleotide barcode from a known set of barcodes with each of the plurality of targets, wherein associating an oligonucleotide barcode with each of the plurality of targets includes contacting each of the targets with a specific binding reagent, wherein the specific binding reagent includes an oligonucleotide barcode; ii) detecting (e.g., sequencing) each barcode to obtain a multiplexed signal in the cell in situ; iii) demultiplexing the multiplexed signal by comparison with the known set of barcodes; and iv) detecting the plurality of targets by identifying the associated barcodes detected in the cell.

In embodiments, the targets are proteins or carbohydrates. In embodiments, the targets are proteins. In embodiments, the targets are carbohydrates. In embodiments when the target are proteins and/or carbohydrates, the method includes contacting the proteins with a specific binding reagent, wherein the specific binding reagent comprises an oligonucleotide barcode. In embodiments, the specific binding reagent comprises an antibody, single-chain Fv fragment (scFv), antibody fragment-antigen binding (Fab), or an aptamer. In embodiments, the specific binding reagent is a peptide, a cell penetrating peptide, an aptamer, a DNA aptamer, an RNA aptamer, an antibody, an antibody fragment, a light chain antibody fragment, a single-chain variable fragment (scFv), a lipid, a lipid derivative, a phospholipid, a fatty acid, a triglyceride, a glycerolipid, a glycerophospholipid, a sphingolipid, a saccharolipid, a polyketide, a polylysine, polyethyleneimine, diethylaminoethyl (DEAE)-dextran, cholesterol, or a sterol moiety. In embodiments, the specific binding reagent interacts (e.g., contacts, or binds) with one or more specific binding reagents on the cell surface. Carbohydrate-specific antibodies are known in the art, see for example Kappler, K., Hennet, T. Genes Immun 21, 224-239 (2020).

In embodiments, associating an oligonucleotide barcode with each of the plurality of targets includes contacting each of the targets with a specific binding reagent, wherein the specific binding reagent includes a circular polynucleotide which includes the oligonucleotide barcode. For example, in embodiments the specific binding reagent is bound (e.g., covalently linked via a tethered capture oligonucleotide, wherein the capture oligonucleotide is hybridized to the circular polynucleotide) to a circular polynucleotide before contacting the target. In embodiments, the specific binding reagent includes an antibody, single-chain Fv fragment (scFv), antibody fragment-antigen binding (Fab), or an aptamer. In embodiments, the specific binding reagent is an antibody, single-chain Fv fragment (scFv), antibody fragment-antigen binding (Fab), or an aptamer. In embodiments, the circular polynucleotide includes a primer binding sequence from a known set of primer binding sequences.

In embodiments, the known set of primer binding sequences includes at least 2 different primer binding sequences. In embodiments, the known set of primer binding sequences includes two or more different primer binding sequences. In embodiments, the known set of primer binding sequences includes at least 3 different primer binding sequences. In embodiments, the known set of primer binding sequences includes three or more different primer binding sequences. In embodiments, the known set of primer binding sequences includes at least 2 different sequencing primer binding sequences. In embodiments, the known set of primer binding sequences includes two or more different sequencing primer binding sequences. In embodiments, the known set of primer binding sequences includes 2 to 10 different sequencing primer binding sequences. In embodiments, the known set of primer binding sequences includes 2 to 6 different sequencing primer binding sequences. In embodiments, the known set of primer binding sequences includes 3 to 8 different sequencing primer binding sequences.

In embodiments the target is an RNA transcript. In embodiments the target is a single stranded RNA nucleic acid sequence. In embodiments, the target is an RNA nucleic acid sequence or a DNA nucleic acid sequence (e.g., cDNA). In embodiments, the target is a cDNA target nucleic acid sequence and before step i), the RNA nucleic acid sequence is reverse transcribed to generate the cDNA target nucleic acid sequence. In embodiments, reverse transcription of the RNA nucleic acid is performed with a reverse transcriptase, for example, Tth DNA polymerase or mutants thereof. In embodiments, the target is genomic DNA (gDNA), mitochondrial DNA, chloroplast DNA, episomal DNA, viral DNA, or copy DNA (cDNA). In embodiments, the target is coding RNA such as messenger RNA (mRNA), and non-coding RNA (ncRNA) such as transfer RNA (tRNA), microRNA (miRNA), small nuclear RNA (snRNA), or ribosomal RNA (rRNA). In embodiments, the target is a cancer-associated gene. In embodiments, to minimize amplification errors or bias, the target is not reverse transcribed to generate cDNA.

In embodiments, the target is an RNA nucleic acid sequence or DNA nucleic acid sequence. In embodiments, the target is an RNA nucleic acid sequence or DNA nucleic acid sequence from the same cell. In embodiments, the target is an RNA nucleic acid sequence. In embodiments, the RNA nucleic acid sequence is stabilized using known techniques in the art. For example, RNA degradation by RNase should be minimized using commercially available solutions, e.g., RNA Later®, RNA Lysis Buffer, or Keratinocyte serum-free medium). In embodiments, the target is messenger RNA (mRNA), transfer RNA (tRNA), micro RNA (miRNA), small interfering RNA (siRNA), small nucleolar RNA (snoRNA), small nuclear RNA (snRNA), Piwi-interacting RNA (piRNA), enhancer RNA (eRNA), or ribosomal RNA (rRNA). In embodiments, the target is pre-mRNA. In embodiments, the target is heterogeneous nuclear RNA (hnRNA). In embodiments, the target is mRNA, tRNA (transfer RNA), rRNA (ribosomal RNA), or noncoding RNA (such as lncRNA (long noncoding RNA)). In embodiments, the targets are on different regions of the same RNA nucleic acid sequence. In embodiments, the targets are cDNA target nucleic acid sequences and before step i), the RNA nucleic acid sequences are reverse transcribed to generate the cDNA target nucleic acid sequences. In embodiments, reverse transcription of the RNA nucleic acid is performed with a reverse transcriptase, for example, Tth DNA polymerase or mutants thereof. In embodiments, the targets are not reverse transcribed to cDNA, i.e., the oligonucleotide primer is hybridized directly to the target nucleic acid.

In embodiments, the methods and compositions described herein are utilized to analyze the various sequences of TCRs and BCRs from immune cells, for example various clonotypes. In embodiments, the target nucleic acid includes a nucleic acid sequence encoding a TCR alpha chain, a TCR beta chain, a TCR delta chain, a TCR gamma chain, or any fragment thereof (e.g., variable regions including VDJ or VJ regions, constant regions, transmembrane regions, fragments thereof, combinations thereof, and combinations of fragments thereof). In embodiments, the target nucleic acid includes a nucleic acid sequence encoding a B cell receptor heavy chain, B cell receptor light chain, or any fragment thereof (e.g., variable regions including VDJ or VJ regions, constant regions, transmembrane regions, fragments thereof, combinations thereof, and combinations of fragments thereof). In embodiments, the target nucleic acid includes a CDR3 nucleic acid sequence. In embodiments, the target nucleic acid includes a TCRA gene sequence or a TCRB gene sequence. In embodiments, the target nucleic acid includes a TCRA gene sequence and a TCRB gene sequence. In embodiments, the target nucleic acid includes sequences of various T cell receptor alpha variable genes (TRAV genes), T cell receptor alpha joining genes (TRAJ genes), T cell receptor alpha constant genes (TRAC genes), T cell receptor beta variable genes (TRBV genes), T cell receptor beta diversity genes (TRBD genes), T cell receptor beta joining genes (TRBJ genes), T cell receptor beta constant genes (TRBC genes), T cell receptor gamma variable genes (TRGV genes), T cell receptor gamma joining genes (TRGJ genes), T cell receptor gamma constant genes (TRGC genes), T cell receptor delta variable genes (TRDV genes), T cell receptor delta diversity genes (TRDD genes), T cell receptor delta joining genes (TRDJ genes), or T cell receptor delta constant genes (TRDC genes).

In embodiments, the target nucleic acid molecule includes a gene sequence, or a fragment thereof. In embodiments, a portion of the entire gene sequence is targeted with one or more of the probes described herein. In embodiments, the target nucleic acid molecule includes a sequence capable of encoding a protein (e.g., a protein imbued with biological function after transcribing and/or translating the nucleic acid sequence).

In embodiments, the target nucleic acid molecule includes an NEUROG3, PYY, MUC2, BEST4, INS, RBP2, MLN, FABP1, SST, OLFM4, FGB, GIP, CCL20, SH2D6, DEFA5, POU2F3, LGR5, CLU, NEUROD1, PHGR1, CCK, BEST2, GRHL1, CKB, NTS, CPB1, CEACAM1, SYTL2, ITLN1, RUNX1, GREM1, WNT5B, CDX2, TPH1, MUC1, MKI67, CDC25C, GREM2, SLC7A5, THBS1, KLF1, REG4, NOVA1, IL18R1, WNT2B, GPX2, CENPK, PAX2, POU2AF1, KRT20, CYP2A6, ACSS3, CNTNAP2, CTNNA2, CTNNB1, CHRM2, RGMB, SMOC2, TICRR, GUCA2A, SCGN, CLCA4, HNF4A, ZNF800, APC, CDH19, CLCA1, JAK3, CLDN1, CDH1, CHGA, CA7, SLC3A2, EPCAM, ANO1, HOTAIR, CLDN3, SLC6A19, CLDN4, IER3, REG1A, TOP2A, ST14, CA2, FAM210B, PLAT, ELF3, SLC16A1, POLD2, CEACAM5, EPHB3, S100P, DSP, LYZ, MUC12, GPRC5A, TFF3, MUC6, TSPAN8, AQP3, RRM2, PYGB, MUC5B, CAMK2N1, DDIT4, TCF7, ODC1, PON2, SPINK4, PROM1, ANXA1, ANXA13, BRAF, FZD7, IGFBP7, MYC, NONO, PLXND1, PROX1, ROBO1, ROBO2, SETD5, SKA3, or TP53 gene sequence.

In embodiments, the nucleic acid molecule includes a gene sequence selected from the group consisting of ACKR1, ACKR4, ACSS3, ACTA2, ADIPOQ, ANGPT2, ANKRD29, ANO1, ANXA1, ANXA13, APC, AQP1, AQP3, BEST2, BEST4, BRAF, CA2, CA7, CAMK2N1, CAVIN2, CCK, CCL2, CCL20, CCL3, CCL4, CCL5, CCR2, CCR5, CCR7, CD14, CD163, CD19, CD1C, CD2, CD247, CD27, CD274, CD276, CD28, CD33, CD34, CD36, CD38, CD3D, CD3E, CD4, CD40, CD40LG, CD44, CD47, CD68, CD70, CD74, CD79A, CD80, CD86, CD8A, CDC25C, CDH1, CDH19, CDX2, CEACAM1, CEACAM5, CEACAM8, CENPK, CHGA, CHRM2, CKB, CLCA1, CLCA4, CLDN1, CLDN3, CLDN4, CLEC9A, CLU, CNTNAP2, COL1A1, CPB1, CSF1R, CSPG4, CTLA4, CTNNA2, CTNNB1, CTSS, CX3CL1, CX3CR1, CXCL10, CXCL11, CXCL13, CXCL9, CXCR3, CXCR4, CXCR5, CXCR6, CYP1A1, CYP2A6, DDIT4, DEFA5, DES, DGKG, DPT, DSP, EGFR, ELF3, EOMES, EPCAM, EPHB3, ERBB2, ESM1, FABP1, FAM210B, FAP, FAS, FASN, FBLN1, FBN1, FCAR, FCGR1A, FCGR3A, FGB, FGFR4, FN1, FOXP3, FSCN1, FZD7, GATA3, GATM, GIP, GNLY, GPC1, GPR183, GPRC5A, GPX2, GREM1, GREM2, GRHL1, GUCA2A, GZMA, GZMB, GZMH, GZMK, HAVCR2, HLA-A, HLA-DRA, HNF4A, HOXD8, ICOSLG, ID2, IDO1, IER3, IFNG, IGFBP7, IGHA1, IGHD, IGHG1, IGHM, IL10, IL10RA, IL17A, IL18R1, IL1B, IL2RA, IL2RB, IL6, IL7R, INS, IRF1, ITGAM, ITGAX, ITGB2, ITLN1, JAK3, JCHAIN, KDR, KIT, KLF1, KLRB1, KLRD1, KLRF1, KLRK1, KRAS, KRT20, LAG3, LAMC3, LARS1, LGR5, LPL, LTBP2, LUM, LYVE1, LYZ, MADCAM1, MAPK1, MET, MKI67, MLN, MMP1, MMRN1, MMRN2, MRC1, MS4A1, MUC1, MUC12, MUC2, MUC5B, MUC6, MYC, MYH11, NCAM1, NCR1, NEUROD1, NEUROG3, NKG7, NONO, NOVA1, NRXN1, NTS, ODC1, OLFM4, PAX2, PDCD1, PDCD1LG2, PDE4A, PDGFRA, PDGFRB, PDK4, PECAMI, PHGR1, PIM1, PLAT, PLIN1, PLXND1, POLD2, POLR2A, PON2, POSTN, POU2AF1, POU2F3, PPARG, PRDM1, PRF1, PROM1, PROX1, PTGS2, PYGB, PYY, RARRES1, RBP2, REG1A, REG4, RGMB, RGS5, ROBO1, ROBO2, RRM2, RSPO3, RUNX1, S100A9, S100P, SCGN, SDC1, SELE, SELL, SETD5, SH2D6, SKA3, SLC16A1, SLC2A1, SLC3A2, SLC6A19, SLC7A5, SMOC2, SNCA, SNCG, SOX10, SPINK4, SST, ST14, STAT1, STAT3, STAT4, STC1, SYTL2, TAGLN, TAP1, TAP2, TBX21, TCF7, TCL1A, TFF3, TFP1, TGFB1, THBS1, THY1, TICRR, TIGIT, TLR2, TLR4, TLR9, TNF, TNFRSF17, TNFRSF4, TNFRSF9, TNFSF13B, TNFSF9, TOP2A, TP53, TPH1, TSPAN8, VCAM1, VCAN, VEGFA, VIM, VWF, WARS1, WNT2B, WNT5B, and ZNF800. In embodiments, the nucleic acid molecule includes a gene sequence selected from the group consisting of TNF, IL1B, IL6, IL17A, IL10, IL10RA, IL23R, IFNG, CXCL9, CXCL10, CXCL11, CXCR3, CCR7, CD3E, CD4, CD8A, FOXP3, CD68, MS4A1, ICOSLG, MUC2, CLDN1, EPCAM, CEACAM5, KRT20, COL1A1, TGFB1, and VCAM1.

RNA, including mRNA, is highly susceptible to degradation upon exposure to one or more RNAses. RNAses are present in a wide range of locations, including water, many reagents, laboratory equipment and surfaces, skin, and mucous membranes. Working with RNA often requires preparing an RNAse-free environment and materials, as well as taking precautions to avoid introducing RNAses into an RNAse-free environment. These precautions include, but are not limited to, cleaning surfaces with an RNAse cleaning product (e.g., RNASEZAP™ and other commercially available products or 0.5% sodium dodecyl sulfate [SDS] followed by 3% H₂O₂); using a designated workspace, materials, and equipment (e.g., pipets, pipet tips); using barrier tips; baking designated glassware (e.g., 300° C. for 2 hours) prior to use; treating enzymes, reagents, and other solutions (e.g., with diethyl pyrocarbonate [DEPC] or dimethyl pyrocarbonate [DMPC]) or using commercially available, certified RNAse-free water or solutions, or ultrafiltered water (e.g., for Tris-based solutions); including an RNAse inhibitor while avoiding temperatures or denaturing conditions that could deactivate the inhibitor); and wearing clean gloves (while avoiding contaminated surfaces) and a clean lab coat.

In embodiments, the cell is an immune cell. In embodiments, the immune cell is a granulocyte, a mast cell, a monocyte, a neutrophil, a dendritic cell, or a natural killer (NK) cell. In embodiments, the immune cell is an adaptive cell, such as a T cell, NK cell, or a B cell. In embodiments, the cell includes a T cell receptor gene sequence, a B cell receptor gene sequence, or an immunoglobulin gene sequence. In embodiments, the immune cell is a granulocyte. In embodiments, the immune cell is a mast cell. In embodiments, the immune cell is a monocyte. In embodiments, the immune cell is a neutrophil. In embodiments, the immune cell is a dendritic cell. In embodiments, the immune cell is a natural killer (NK) cell. In embodiments, the immune cell is a T cell. In embodiments, the immune cell is a B cell. In embodiments, the cell includes a T cell receptor gene sequence. In embodiments, the cell includes a B cell receptor gene sequence. In embodiments, the cell includes an immunoglobulin gene sequence. In embodiments, the plurality of target nucleic acids includes non-contiguous regions of a nucleic acid molecule. In embodiments, the non-contiguous regions include regions of a VDJ recombination of a B cell or T cell. In embodiments, the cell is an adherent cell (e.g., epithelial cell, endothelial cell, or neural cell). Adherent cells are usually derived from tissues of organs and attach to a substrate (e.g., epithelial cells adhere to an extracellular matrix coated substrate via transmembrane adhesion protein complexes). Adherent cells typically require a substrate, e.g., tissue culture plastic, which may be coated with extracellular matrix (e.g., collagen and laminin) components to increase adhesion properties and provide other signals needed for growth and differentiation. In embodiments, the cell is a neuronal cell, an endothelial cell, epithelial cell, germ cell, plasma cell, a muscle cell, peripheral blood mononuclear cell (PBMC), a myocardial cell, or a retina cell. In embodiments, the cell is a suspension cell (e.g., a cell free-floating in the culture medium, such a lymphoblast or hepatocyte). In embodiments, the cell is a glial cell (e.g., astrocyte, radial glia), pericyte, or stem cell (e.g., a neural stem cell). In embodiments, the cell is a neuronal cell. In embodiments, the cell is an endothelial cell. In embodiments, the cell is an epithelial cell. In embodiments, the cell is a germ cell. In embodiments, the cell is a plasma cell. In embodiments, the cell is a muscle cell. In embodiments, the cell is a peripheral blood mononuclear cell (PBMC). In embodiments, the cell is a myocardial cell. In embodiments, the cell is a retina cell. In embodiments, the cell is a lymphoblast. In embodiments, the cell is a hepatocyte. In embodiments, the cell is a glial cell. In embodiments, the cell is an astrocyte. In embodiments, the cell is a radial glia. In embodiments, the cell is a pericyte. In embodiments, the cell is a stem cell. In embodiments, the cell is a neural stem cell. In embodiments, the cell is a cancer cell.

In embodiments, the cancer cell includes a cancer-associated gene (e.g., an oncogene associated with kinases and genes involved in DNA repair) or a cancer-associated biomarker. A “biomarker” is a substance that is associated with a particular characteristic, such as a disease or condition. A change in the levels of a biomarker may correlate with the risk or progression of a disease or with the susceptibility of the disease to a given treatment. In embodiments, the cancer is Acute Myeloid Leukemia, Adrenocortical Carcinoma, Bladder Urothelial Carcinoma, Breast Ductal Carcinoma, Breast Lobular Carcinoma, Cervical Carcinoma, Cholangiocarcinoma, Colorectal Adenocarcinoma, Esophageal Carcinoma, Gastric Adenocarcinoma, Glioblastoma Multiforme, Head and Neck Squamous Cell Carcinoma, Hepatocellular Carcinoma, Kidney Chromophobe Carcinoma, Kidney Clear Cell Carcinoma, Kidney Papillary Cell Carcinoma, Lower Grade Glioma, Lung Adenocarcinoma, Lung Squamous Cell Carcinoma, Mesothelioma, Ovarian Serous Adenocarcinoma, Pancreatic Ductal Adenocarcinoma, Paraganglioma & Pheochromocytoma, Prostate Adenocarcinoma, Sarcoma, Skin Cutaneous Melanoma, Testicular Germ Cell Cancer, Thymoma, Thyroid Papillary Carcinoma, Uterine Carcinosarcoma, Uterine Corpus Endometrioid Carcinoma, or Uveal Melanoma. In embodiments, the cancer-associated gene is a nucleic acid sequence identified within The Cancer Genome Atlas Program.

In embodiments, the cell is a cell (e.g., a T cell) within a tumor. In embodiments, the cell is a non-allogenic cell (i.e., native cell to the subject) within a tumor. In embodiments, the cell is a tumor infiltrating lymphocyte (TIL). In embodiments, the cell is an allogenic cell. In embodiments, the cell is a circulating tumor cell.

In embodiments, the cell in situ is obtained from a subject (e.g., human or animal tissue). Once obtained, the cell is placed in an artificial environment in plastic or glass containers supported with specialized medium containing essential nutrients and growth factors to support proliferation. In embodiments, the cell is permeabilized and immobilized to a solid support surface. In embodiments, the cell is permeabilized and immobilized to an array (i.e., to discrete locations arranged in an array). In embodiments, the cell is immobilized to a solid support surface. In embodiments, the surface includes a patterned surface (e.g., suitable for immobilization of a plurality of cells in an ordered pattern. The discrete regions of the ordered pattern may have defined locations in a regular array, which may correspond to a rectilinear pattern, circular pattern, hexagonal pattern, or the like. These discrete regions are separated by interstitial regions. As used herein, the term “interstitial region” refers to an area in a substrate or on a surface that separates other areas of the substrate or surface. In embodiments, a plurality of cells are immobilized on a patterned surface that have a mean or median separation from one another of about 10-20 μm. In embodiments, a plurality of cells are immobilized on a patterned surface that have a mean or median separation from one another of about 10-20; 10-50; or 100 μm. In embodiments, a plurality of cells are arrayed on a substrate. In embodiments, a plurality of cells are immobilized in a 96-well microplate having a mean or median well-to-well spacing of about 8 mm to about 12 mm (e.g., about 9 mm). In embodiments, a plurality of cells are immobilized in a 384-well microplate having a mean or median well-to-well spacing of about 3 mm to about 6 mm (e.g., about 4.5 mm).

In embodiments, the method includes immobilizing a plurality of tissue sections to the solid support (e.g., a flow cell), wherein a tissue in a plurality of tissue sections includes the biomolecule to be detected. In embodiments, the method includes immobilizing 24 tissue sections (10 mm×17 mm sections). In embodiments, the method includes immobilizing 40 tissue sections (10 mm×10 mm sections). In embodiments, the method includes immobilizing 128 tissue sections (4 mm×4 mm sections).

The cell or tissue may be manipulated prior to immobilizing the cell or tissue onto a solid support using known techniques in the art (see, e.g., PCT Publication WO2023076832A1). In embodiments, the method further includes cutting a sample portion from the biological sample (e.g., including cells or tissues) using a punch device such that the punch device contains the sample portion; mounting the punch device containing the sample portion onto the first solid support as described herein (e.g., inverting the punch device); pushing the sample portion out of the punch device using a piston, so that all or a portion thereof of the sample portion is positioned on the first solid support as described herein. In embodiments, the method further includes cutting a sample portion from the biological sample using two or more punch devices such that each punch device contains a different the sample portion; mounting each punch device containing the sample portion onto the first solid support as described herein; pushing the sample portions out of the punch devices using one or more pistons so that the sample portions are positioned onto the solid support as described herein (e.g., a flow cell described herein).

In embodiments, the cell is attached to the substrate via a bioconjugate reactive linker. In embodiments, the cell is attached to the substrate via a specific binding reagent. In embodiments, the specific binding reagent includes an antibody, single-chain Fv fragment (scFv), antibody fragment-antigen binding (Fab), or an aptamer. In embodiments, the specific binding reagent includes an antibody, or antigen binding fragment, an aptamer, affimer, or non-immunoglobulin scaffold. In embodiments, the specific binding reagent is a peptide, a cell penetrating peptide, an aptamer, a DNA aptamer, an RNA aptamer, an antibody, an antibody fragment, a light chain antibody fragment, a single-chain variable fragment (scFv), a lipid, a lipid derivative, a phospholipid, a fatty acid, a triglyceride, a glycerolipid, a glycerophospholipid, a sphingolipid, a saccharolipid, a polyketide, a polylysine, polyethyleneimine, diethylaminoethyl (DEAE)-dextran, cholesterol, or a sterol moiety. Substrates may be prepared for selective capture of particular cells. For example, a substrate containing a plurality of bioconjugate reactive moieties or a plurality of specific binding reagents, optionally in an ordered pattern, contacts a plurality of cells. Only cells containing complementary bioconjugate reactive moieties or complementary specific binding reagents are capable of reacting, and thus adhering, to the substrate.

In embodiments, the methods are performed in situ on isolated cells or in tissue sections that have been prepared according to methodologies known in the art. Methods for permeabilization and fixation of cells and tissue samples are known in the art, as exemplified by Cremer et al., The Nucleus: Volume 1: Nuclei and Subnuclear Components, R. Hancock (ed.) 2008; and Larsson et al., Nat. Methods (2010) 7:395-397, the content of each of which is incorporated herein by reference in its entirety. In embodiments, the cell is cleared (e.g., digested) of proteins, lipids, or proteins and lipids.

In embodiments, the cell is immobilized to a substrate. The cell may have been cultured on the surface, or the cell may have been initially cultured in suspension and then fixed to the surface. Substrates can be two- or three-dimensional and can include a planar surface (e.g., a glass slide). A substrate can include glass (e.g., controlled pore glass (CPG)), quartz, plastic (such as polystyrene (low cross-linked and high cross-linked polystyrene), polycarbonate, polypropylene and poly(methymethacrylate)), acrylic copolymer, polyamide, silicon, metal (e.g., alkanethiolate-derivatized gold), cellulose, nylon, latex, dextran, gel matrix (e.g., silica gel), polyacrolein, or composites. In embodiments, the substrate includes a polymeric coating, optionally containing bioconjugate reactive moieties capable of affixing the sample. Suitable three-dimensional substrates include, for example, spheres, microparticles, beads, membranes, slides, plates, micromachined chips, tubes (e.g., capillary tubes), microwells, microfluidic devices, channels, filters, or any other structure suitable for anchoring a sample. In embodiments, the substrate is not a flow cell. In embodiments, the substrate includes a polymer matrix material (e.g., polyacrylamide, cellulose, alginate, polyamide, cross-linked agarose, cross-linked dextran or cross-linked polyethylene glycol), which may be referred to herein as a “matrix”, “synthetic matrix”, “exogenous polymer” or “exogenous hydrogel”. In embodiments, a matrix may refer to the various components and organelles of a cell, for example, the cytoskeleton (e.g., actin and tubulin), endoplasmic reticulum, Golgi apparatus, vesicles, etc. In embodiments, the matrix is endogenous to a cell. In embodiments, the matrix is exogenous to a cell. In embodiments, the matrix includes both the intracellular and extracellular components of a cell. In embodiments, polynucleotide primers may be immobilized on a matrix including the various components and organelles of a cell. Immobilization of polynucleotide primers on a matrix of cellular components and organelles of a cell is accomplished as described herein, for example, through the interaction/reaction of complementary bioconjugate reactive moieties. In embodiments, the exogenous polymer may be a matrix or a network of extracellular components that act as a point of attachment (e.g., act as an anchor) for the cell to a substrate.”

In embodiments, the cell is exposed to paraformaldehyde (i.e., by contacting the cell with paraformaldehyde). Any suitable permeabilization and fixation technologies can be used for making the cell available for the detection methods provided herein. In embodiments the method includes affixing single cells or tissues to a transparent substrate. Exemplary tissue include those from skin tissue, muscle tissue, bone tissue, organ tissue and the like. In embodiments, the method includes immobilizing the cell in situ to a substrate and permeabilized for delivering probes, enzymes, nucleotides and other components required in the reactions. In embodiments, the cell includes many cells from a tissue section in which the original spatial relationships of the cells are retained. In embodiments, the cell in situ is within a Formalin-Fixed Paraffin-Embedded (FFPE) sample. In embodiments, the cell is subjected to paraffin removal methods, such as methods involving incubation with a hydrocarbon solvent, such as xylene or hexane, followed by two or more washes with decreasing concentrations of an alcohol, such as ethanol. The cell may be rehydrated in a buffer, such as PBS, TBS or MOPs. In embodiments, the FFPE sample is incubated with xylene and washed using ethanol to remove the embedding wax, followed by treatment with Proteinase K to permeabilized the tissue. In embodiments, the cell is fixed with a chemical fixing agent. In embodiments, the chemical fixing agent is formaldehyde or glutaraldehyde. In embodiments, the chemical fixing agent is glyoxal or dioxolane. In embodiments, the chemical fixing agent includes one or more of ethanol, methanol, 2-propanol, acetone, and glyoxal. In embodiments, the chemical fixing agent includes formalin, Greenfix®, Greenfix® Plus, UPM, CyMol®, HOPE®, CytoSkelFix™, F-Solv®, FineFIX®, RCL2/KINFix, UMFIX, Glyo-Fixx®, Histochoice®, or PAXgene®. In embodiments, the cell is fixed within a synthetic three-dimensional matrix (e.g., polymeric material). In embodiments, the synthetic matrix includes polymeric-crosslinking material. In embodiments, the material includes polyacrylamide, poly-ethylene glycol (PEG), poly(acrylate-co-acrylic acid) (PAA), or Poly(N-isopropylacrylamide) (NIPAM).

In embodiments, the oligonucleotide primer (alternatively referred to as a polynucleotide probe, or padlock probe) is a single-stranded polynucleotide having at least one primer binding sequence. In embodiments, the oligonucleotide primer includes at least one amplification primer binding sequence and at least one sequencing primer binding sequence. In embodiments, the oligonucleotide primer includes at least two primer binding sequences. In embodiments, the oligonucleotide primer includes an amplification primer binding sequence. In embodiments, the oligonucleotide primer includes a sequencing primer binding sequence. The amplification primer binding sequence refers to a nucleotide sequence that is complementary to a primer useful in initiating amplification (i.e., an amplification primer). Likewise, a sequencing primer binding sequence is a nucleotide sequence that is complementary to a primer useful in initiating sequencing (i.e., a sequencing primer). Primer binding sequences usually have a length in the range of between 3 to 36 nucleotides, also 5 to 24 nucleotides, also from 14 to 36 nucleotides. In embodiments, the oligonucleotide primer does not include a barcode. In embodiments, an amplification primer and a sequencing primer are complementary to the same primer binding sequence, or overlapping primer binding sequences. In embodiments, an amplification primer and a sequencing primer are complementary to different primer binding sequences. In embodiments, the primer binding sequence is complementary to a fluorescent in situ hybridization (FISH) probe. FISH probes may be custom designed using known techniques in the art, see for example Gelali, E., Girelli, G., Matsumoto, M. et al. Nat Commun 10, 1636 (2019).

In embodiments, the oligonucleotide primer (alternatively referred to herein as a polynucleotide probe) is approximately 50 to 200 nucleotides. In embodiments, the oligonucleotide primer has a first domain that is capable of hybridizing to a first target sequence domain, and a second ligation domain, capable of hybridizing to a target nucleic acid sequence-adjacent second sequence domain. In embodiments, following hybridization there is a gap between the first target sequence domain, and the second ligation domain, wherein the gap spans the length of the target nucleic acid sequence. In embodiments, the oligonucleotide primer has a first domain that is capable of hybridizing to a first target sequence domain, and a second domain capable of hybridizing to a second target sequence domain. In embodiments, the length of the first domain and second domain are the same length (e.g., both the first and the second domains are about 15 nucleotides). In embodiments, the length of the first domain and second domain are different lengths (e.g., the first domain is about 10 nucleotides and the second domain is about 20 nucleotides). In embodiments, an asymmetric oligonucleotide primer (i.e., an oligonucleotide primer having a first domain and second domain that are different sequence lengths) may be advantageous in preventing non-specific hybridization. In embodiments, the total length of the first domain and second domain is about 25, 30, 35, or 40 nucleotides. In embodiments, the total length of the first domain and second domain is about 30 nucleotides. In embodiments, the total length of the first domain and second domain is about 15 to 25 nucleotides. In embodiments, the total length of the first domain is about 15 to 25 nucleotides and the total length of the second domain is about 20 to 25 nucleotides. In embodiments, the oligonucleotide primer includes at least one target-specific region. In embodiments, the oligonucleotide primer includes two target-specific regions. In embodiments, the oligonucleotide primer includes at least one flanking-target region (i.e., an oligonucleotide sequence that flanks the region of interest). In embodiments, the oligonucleotide primer includes two flanking-target regions. A target-specific region is a single stranded polynucleotide that is at least 50% complementary, at least 75% complementary, at least 85% complementary, at least 90% complementary, at least 95% complementary, at least 98%, at least 99% complementary, or 100% complementary to a portion of a nucleic acid molecule that includes a target sequence (e.g., a gene of interest). In embodiments, the target-specific region is capable of hybridizing to at least a portion of the target sequence. In embodiments, the target-specific region is substantially non-complementary to other target sequences present in the sample.

In embodiments, each oligonucleotide (e.g., one or more oligonucleotides of a plurality of oligonucleotides) includes locked nucleic acids (LNAs), Bis-locked nucleic acids (bisLNAs), twisted intercalating nucleic acids (TINAs), bridged nucleic acids (BNAs), 2′-O-methyl RNA:DNA chimeric nucleic acids, minor groove binder (MGB) nucleic acids, morpholino nucleic acids, C5-modified pyrimidine nucleic acids, peptide nucleic acids (PNAs), or combinations thereof. In embodiments, the first oligonucleotide, the second oligonucleotide, and/or the circularizable oligonucleotide includes locked nucleic acids (LNAs), Bis-locked nucleic acids (bisLNAs), twisted intercalating nucleic acids (TINAs), bridged nucleic acids (BNAs), 2′-O-methyl RNA:DNA chimeric nucleic acids, minor groove binder (MGB) nucleic acids, morpholino nucleic acids, C5-modified pyrimidine nucleic acids, peptide nucleic acids (PNAs), or combinations thereof.

In embodiments, each oligonucleotide includes one or more locked nucleic acid (LNA) nucleotides. In embodiments, the target hybridization sequence of each oligonucleotide includes one or more LNA nucleotides. In embodiments, the first hybridization sequence and/or the second hybridization sequence of each oligonucleotide includes one or more LNA nucleotides. In embodiments, the first target hybridization sequence of the first oligonucleotide and/or the second target hybridization sequence of the second oligonucleotide include one or more LNA nucleotides. In embodiments, the sequence complementary to the first hybridization sequence and/or the second sequence complementary to the second hybridization sequence of the circularizable oligonucleotide includes one or more LNA nucleotides. In embodiments, the oligonucleotide includes 1, 2, 3, 4, 5, 6, 7, 8, 9, or 10 LNAs.

In embodiments, the target hybridization sequence of the oligonucleotide includes a plurality of LNAs interspersed throughout the target hybridization sequence. In embodiments, the hybridization sequence (or complement thereof) of the oligonucleotide and/or circularizable oligonucleotide includes a plurality of LNAs interspersed throughout the hybridization sequence, or complement thereof.

In embodiments, the target hybridization sequence and/or hybridization sequence (e.g., the first hybridization sequence, the second hybridization sequence, and/or the third hybridization sequence) includes Bis-locked nucleic acids (bisLNAs). In embodiments, the target hybridization sequence and/or hybridization sequence includes twisted intercalating nucleic acids (TINAs). In embodiments, the target hybridization sequence and/or hybridization sequence includes bridged nucleic acids (BNAs). In embodiments, the target hybridization sequence and/or hybridization sequence includes 2′-O-methyl RNA:DNA chimeric nucleic acids. In embodiments, the target hybridization sequence and/or hybridization sequence includes minor groove binder (MGB) nucleic acids. In embodiments, the target hybridization sequence and/or hybridization sequence includes morpholino nucleic acids. Morpholino nucleic acids are synthetic nucleotides that have standard nucleic acid bases (e.g., adenine, guanine, cytosine, and thymine) wherein those bases are bound to methylenemorpholine rings linked through phosphorodiamidate groups instead of phosphates. Morpholino nucleic acids may be referred to as phosphorodiamidate morpholino oligomers (PMOs). In embodiments, the target hybridization sequence and/or hybridization sequence includes C5-modified pyrimidine nucleic acids. In embodiments, the target hybridization sequence and/or hybridization sequence includes peptide nucleic acids (PNAs). In embodiments, the target hybridization sequence and/or hybridization sequence includes from 5′ to 3′ a plurality of synthetic nucleotides (e.g., LNAs) followed by a plurality (e.g., 2 to 5) canonical or native nucleotides (e.g., dNTPs). In embodiments, the target hybridization sequence and/or hybridization sequence includes one or more (e.g., 2 to 5) deoxyuracil nucleobases (dU). In embodiments, the one or more dU nucleobases are at or near the 3′ end of the target hybridization sequence and/or hybridization sequence (e.g., within 5 nucleotides of the 3′ end). In embodiments, the target hybridization sequence and/or hybridization sequence includes from 5′ to 3′ a plurality (e.g., 2 to 5) of phosphorothioate nucleic acids, followed by a plurality of synthetic nucleotides (e.g., LNAs), and subsequently followed by a plurality (e.g., 2 to 5) of canonical nucleobases. In some embodiments, the target hybridization sequence and/or hybridization sequence includes a plurality of canonical nucleobases, wherein the canonical nucleobases terminate (i.e., at the 3′ end) with a deoxyuracil nucleobase (dU).

In embodiments, the target hybridization sequence and/or hybridization sequence (e.g., the first hybridization sequence, the second hybridization sequence, and/or the third hybridization sequence) includes a plurality of LNAs interspersed throughout the polynucleotide. In embodiments, the target hybridization sequence and/or hybridization sequence includes a plurality of consecutive LNAs (e.g., 2 to 5 LNAs, 5 to 7 LNAs, or 7 to 10 LNAs) throughout the target hybridization sequence and/or hybridization sequence. In embodiments, the entire composition of the target hybridization sequence and/or hybridization sequence includes less than 70%, less than 60%, less than 50%, less than 40%, less than 30%, less than 20%, less than 10%, or less than 5% of LNAs. In embodiments, the entire composition of the target hybridization sequence and/or hybridization sequence includes up to about 70%, up to about 60%, up to about 50%, up to about 40%, up to about 30%, up to about 20%, up to about 10%, or up to about 5% of LNAs. In embodiments, the entire composition of the target hybridization sequence and/or hybridization sequence includes more than 60%, more than 50%, more than 40%, more than 30%, more than 20%, more than 10%, or more than 5% of LNAs. In embodiments, the entire composition of the target hybridization sequence and/or hybridization sequence includes about 5% to about 10%, about 10% to about 20%, about 20% to about 30%, about 30% to about 40%, about 40% to about 50%, about 50% to about 60%, or about 60% to about 70% of LNAs. In embodiments, the entire composition of the target hybridization sequence and/or hybridization sequence includes about 5%, about 10%, about 15%, about 20%, about 25%, about 30%, about 35%, about 40%, about 45%, about 50%, about 55%, about 60%, about 65%, or about 70% of LNAs. In embodiments, the entire composition of the target hybridization sequence and/or hybridization sequence includes about 30%, about 35%, about 40%, about 45%, about 50%, about 55%, about 60%, about 65%, about 70%, about 75%, about 80%, about 85%, about 90%, or about 95% of canonical dNTPs. In embodiments, the entire composition of the target hybridization sequence and/or hybridization sequence includes less than 95%, less than 90%, less than 80%, less than 70%, less than 60%, less than 50%, less than 40%, or less than 30% of canonical dNTPs. In embodiments, the entire composition of the target hybridization sequence and/or hybridization sequence includes up to about 95%, up to about 90%, up to about 80%, up to about 70%, up to about 60%, up to about 50%, up to about 40%, or up to about 30% of canonical dNTPs. In embodiments, the entire composition of the target hybridization sequence and/or hybridization sequence includes more than 90%, more than 80%, more than 70%, more than 60%, more than 50%, more than 40%, or more than 30% of canonical dNTPs.

Padlock probes are specialized ligation probes, examples of which are known in the art, see for example Nilsson M, et al. Science. 1994; 265(5181):2085-2088), and has been applied to detect transcribed RNA in cells, see for example Christian A T, et al. Proc Natl Acad Sci USA. 2001; 98(25):14238-14243, both of which are incorporated herein by reference in their entireties. In embodiments, the padlock probe is approximately 50 to 200 nucleotides. In embodiments, a padlock probe has a first domain that is capable of hybridizing to a first target sequence domain, and a second ligation domain, capable of hybridizing to an adjacent second sequence domain. The configuration of the padlock probe is such that upon ligation of the first and second ligation domains of the padlock probe, the probe forms a circular polynucleotide, and forms a complex with the sequence (i.e., the sequence it hybridized to, the target sequence) wherein the target sequence is “inserted” into the loop of the circle. Padlock probes are useful for the methods provided herein and include, for example, padlock probes for genomic analyses, as exemplified by Gore, A. et al. Nature 471, 63-67 (2011); Porreca, G. J. et al. Nat Methods 4, 931-936 (2007); Li, J. B. et al. Genome Res 19, 1606-1615 (2009), Zhang, K. et al. Nat Methods 6, 613-618 (2009); Noggle, S. et al. Nature 478, 70-75 (2011); and Li, J. B. et al. Science 324, 1210-1213 (2009), the content of each of which is incorporated by reference in its entirety.

In embodiments, the padlock probe includes at least one target-specific region. A target-specific region is a single stranded polynucleotide that is at least 50% complementary, at least 75% complementary, at least 85% complementary, at least 90% complementary, at least 95% complementary, at least 98%, at least 99% complementary, or 100% complementary to a portion of a nucleic acid molecule that includes a target sequence (e.g., a gene of interest). In embodiments, the target-specific region is capable of hybridizing to at least a portion of the target sequence. In embodiments, the target-specific region is substantially non-complementary to other target sequences present in the sample. In embodiments, the padlock probe includes at two target-specific regions.

In embodiments, the padlock probe has a first domain that is capable of hybridizing to a first target sequence domain, and a second domain capable of hybridizing to an adjacent second target sequence domain. In embodiments, the length of the first domain and second domain are the same length (e.g., both the first and the second domains are about 15 nucleotides). In embodiments, the length of the first domain and second domain are different lengths (e.g., the first domain is about 10 nucleotides and the second domain is about 20 nucleotides). In embodiments, an asymmetric padlock probe (i.e., a padlock probe having a first domain and second domain that are different lengths) may be advantageous in preventing non-specific hybridization. In embodiments, the total length of the first domain and second domain is about 25, 30, 35, or 40 nucleotides. In embodiments, the total length of the first domain and second domain is about 30 nucleotides.

In embodiments, the method includes hybridizing a padlock probe to two adjacent nucleic acid sequences (i.e., no gap) of the target nucleic acid molecule, wherein the padlock probe is a single-stranded polynucleotide having a 5′ and a 3′ end. In embodiments, the target nucleic acid serves as a splint for the padlock probe. In embodiments, the method includes hybridizing a padlock probe to two nucleic acid sequences of the target nucleic acid molecule separated by a target sequence, wherein the padlock probe is a single-stranded polynucleotide having a 5′ and a 3′ end. In embodiments, the target nucleic acid serves as a splint for the padlock probe.

In embodiments, circularizing the oligonucleotide primer to generate a circular oligonucleotide includes extending the 3′ end of the oligonucleotide primer (e.g., extending the 3′ end of the primer using a polymerase (e.g., a Thermus thermophilus (Tth) DNA polymerase) to incorporate one or more nucleotides) along the target nucleic acid to generate a complementary sequence (e.g., complementary to the target nucleic acid, for example a target RNA sequence), and ligating the complementary sequence to the 5′ end of the oligonucleotide primer. In embodiments, the ligation includes enzymatic ligation. In embodiments, ligating includes enzymatic ligation including a ligation enzyme (e.g., Circligase enzyme, Taq DNA Ligase, HiFi Taq DNA Ligase, T4 ligase, PBCV-1 DNA Ligase (also known as SplintR ligase) or Ampligase DNA Ligase). Non-limiting examples of ligases include DNA ligases such as DNA Ligase I, DNA Ligase II, DNA Ligase III, DNA Ligase IV, T4 DNA ligase, T7 DNA ligase, T3 DNA Ligase, E. coli DNA Ligase, PBCV-1 DNA Ligase (also known as SplintR ligase) or a Taq DNA Ligase. In embodiments, the ligase enzyme includes a T4 DNA ligase, T4 RNA ligase 1, T4 RNA ligase 2, T3 DNA ligase or T7 DNA ligase. In embodiments, the enzymatic ligation is performed by a mixture of ligases. In embodiments, the ligation enzyme is selected from the group consisting of T4 DNA ligase, T4 RNA ligase 1, T4 RNA ligase 2, RtcB ligase, T3 DNA ligase, T7 DNA ligase, Taq DNA ligase, PBCV-1 DNA Ligase, a thermostable DNA ligase (e.g., 5′AppDNA/RNA ligase), an ATP dependent DNA ligase, an RNA-dependent DNA ligase (e.g., SplintR ligase), and combinations thereof.

In embodiments, the method further includes an amplification method for amplifying the circular polynucleotide. In embodiments, the method further includes amplifying the circular polynucleotide by extending an amplification primer with a polymerase (e.g., a strand-displacing polymerase), wherein the primer extension generates an extension product including multiple complements of the circular polynucleotide, referred to as an amplicon. An amplicon typically contains multiple, tandem copies of the circularized nucleic acid molecule of the corresponding sample nucleic acid. The number of copies can be varied by appropriate modification of the reaction conditions, such as varying the number of amplification cycles, using polymerases of varying processivity in the amplification reaction, or varying the length of time that the amplification reaction is run. In embodiments, the circular polynucleotide is copied about 5-50 times (i.e., the extension product includes about 5 to 50 complements of the circular polynucleotide). In embodiments, the circular polynucleotide is copied about 100-300 times (i.e., the extension product includes about 100 to 300 complements of the circular polynucleotide). In embodiments, the method includes hybridizing an amplification primer and oligonucleotide primer in the same reaction (e.g., simultaneously). In embodiments, the method includes contacting the target with an amplification primer and oligonucleotide primer in the same reaction (e.g., simultaneously).

In embodiments, the amplifying includes rolling circle amplification (RCA) or rolling circle transcription (RCT) (see, e.g., Lizardi et al., Nat. Genet. 19:225-232 (1998), which is incorporated herein by reference in its entirety). Several suitable rolling circle amplification methods are known in the art. For example, RCA amplifies a circular polynucleotide (e.g., DNA) by polymerase extension of an amplification primer complementary to a portion of the template polynucleotide. This process generates copies of the circular polynucleotide template such that multiple complements of the template sequence arranged end to end in tandem are generated (i.e., a concatemer) locally preserved at the site of the circle formation. In embodiments, the amplifying occurs at isothermal conditions. In embodiments, the amplifying includes hybridization chain reaction (HCR). HCR uses a pair of complementary, kinetically trapped hairpin oligomers to propagate a chain reaction of hybridization events, as described in Dirks, R. M., & Pierce, N. A. (2004) PNAS USA, 101(43), 15275-15278, which is incorporated herein by reference for all purposes. In embodiments, the amplifying includes branched rolling circle amplification (BRCA); e.g., as described in Fan T, Mao Y, Sun Q, et al. Cancer Sci. 2018; 109:2897-2906, which is incorporated herein by reference in its entirety. In embodiments, the amplifying includes hyberbranched rolling circle amplification (HRCA). Hyperbranched RCA uses a second primer complementary to the first amplification product. This allows products to be replicated by a strand-displacement mechanism, which yields drastic amplification within an isothermal reaction (Lage et al., Genome Research 13:294-307 (2003), which is incorporated herein by reference in its entirety). In embodiments, amplifying includes polymerase extension of an amplification primer. In embodiments, the polymerase is T4, T7, Sequenase, Taq, Klenow, and Pol I DNA polymerases. SD polymerase, Bst large fragment polymerase, or a phi29 polymerase or mutant thereof. In embodiments, the strand-displacing enzyme is an SD polymerase, Bst large fragment polymerase, or a phi29 polymerase or mutant thereof. In embodiments, the strand-displacing polymerase is phi29 polymerase, phi29 mutant polymerase or a thermostable phi29 mutant polymerase. A “phi polymerase” (or “Φ29 polymerase”) is a DNA polymerase from the Φ29 phage or from one of the related phages that, like Φ29, contain a terminal protein used in the initiation of DNA replication. For example, phi29 polymerases include the B103, GA-1, PZA, Φ15, BS32, M2Y (also known as M2), Nf, G1, Cp-1, PRD1, PZE, SFS, Cp-5, Cp-7, PR4, PR5, PR722, L17, Φ21, and AV-1 DNA polymerases, as well as chimeras thereof. A phi29 mutant DNA polymerase includes one or more mutations relative to naturally-occurring wild-type phi29 DNA polymerases, for example, one or more mutations that alter interaction with and/or incorporation of nucleotide analogs, increase stability, increase read length, enhance accuracy, increase phototolerance, and/or alter another polymerase property, and can include additional alterations or modifications over the wild-type phi29 DNA polymerase, such as one or more deletions, insertions, and/or fusions of additional peptide or protein sequences.

In embodiments, amplifying the circular oligonucleotide includes incubating the circular oligonucleotide with the strand-displacing polymerase for about 1 hour to about 12 hours. In embodiments, amplifying includes incubation with the strand-displacing polymerase for about 60 seconds to about 60 minutes. In embodiments, amplifying includes incubation with the strand-displacing polymerase for about 10 minutes to about 60 minutes. In embodiments, amplifying includes incubation with the strand-displacing polymerase for about 10 minutes to about 30 minutes. In embodiments, amplifying the circular oligonucleotide includes incubating the circular oligonucleotide with the strand-displacing polymerase for about 1, about 2, about 3, about 4, about 5, about 6, about 7, about 8, about 9, about 10, about 11, or about 12 hours. In embodiments, amplifying the circular oligonucleotide includes incubating the circular oligonucleotide with the strand-displacing polymerase for more than 12 hours.

In embodiments, the extension product includes three or more copies of the target nucleic acid. In embodiments, the extension product includes at least three or more copies of the target nucleic acid. In embodiments, the extension product includes at least five or more copies of the target nucleic acid. In embodiments, the extension product includes at 5 to 10 copies of the target nucleic acid. In embodiments, the extension product includes 10 to 20 copies of the target nucleic acid. In embodiments, the extension product includes 20 to 50 copies of the target nucleic acid.

In embodiments, the method includes sequencing the extension products, which includes the target nucleic acid sequence. A variety of sequencing methodologies can be used such as sequencing-by synthesis (SBS), pyrosequencing, sequencing by ligation (SBL), or sequencing by hybridization (SBH). Pyrosequencing detects the release of inorganic pyrophosphate (PPi) as particular nucleotides are incorporated into a nascent nucleic acid strand (Ronaghi, et al., Analytical Biochemistry 242(1), 84-9 (1996); Ronaghi, Genome Res. 11(1), 3-11 (2001); Ronaghi et al. Science 281(5375), 363 (1998); U.S. Pat. Nos. 6,210,891; 6,258,568; and. 6,274,320, each of which is incorporated herein by reference in its entirety). In pyrosequencing, released PPi can be detected by being converted to adenosine triphosphate (ATP) by ATP sulfurylase, and the level of ATP generated can be detected via light produced by luciferase. In this manner, the sequencing reaction can be monitored via a luminescence detection system. In both SBL and SBH methods, target nucleic acids, and amplicons thereof, are subjected to repeated cycles of oligonucleotide delivery and detection. SBL methods, include those described in Shendure et al. Science 309:1728-1732 (2005); U.S. Pat. Nos. 5,599,675; and 5,750,341, each of which is incorporated herein by reference in its entirety; and the SBH methodologies are as described in Bains et al., Journal of Theoretical Biology 135(3), 303-7 (1988); Drmanac et al., Nature Biotechnology 16, 54-58 (1998); Fodor et al., Science 251(4995), 767-773 (1995); and WO 1989/10977, each of which is incorporated herein by reference in its entirety.

In SBS, extension of a nucleic acid primer along a nucleic acid template is monitored to determine the sequence of nucleotides in the template. The underlying chemical process can be catalyzed by a polymerase, wherein fluorescently labeled nucleotides are added to a primer (thereby extending the primer) in a template dependent fashion such that detection of the order and type of nucleotides added to the primer can be used to determine the sequence of the template. In embodiments, sequencing includes annealing and extending a sequencing primer to incorporate a detectable label that indicates the identity of a nucleotide in the target polynucleotide, detecting the detectable label, and repeating the extending and detecting of steps. In embodiments, the methods include sequencing one or more bases of a target nucleic acid by extending a sequencing primer hybridized to a target nucleic acid (e.g., an amplification product produced by the amplification methods described herein). In embodiments, sequencing may be accomplished by a sequencing-by-synthesis (SBS) process. In embodiments, sequencing includes a sequencing by synthesis process, where individual nucleotides are identified iteratively, as they are polymerized to form a growing complementary strand. In embodiments, nucleotides added to a growing complementary strand include both a label and a reversible chain terminator that prevents further extension, such that the nucleotide may be identified by the label before removing the terminator to add and identify a further nucleotide. Such reversible chain terminators include removable 3′ blocking groups, for example as described in U.S. Pat. Nos. 7,541,444 and 7,057,026. Once such a modified nucleotide has been incorporated into the growing polynucleotide chain complementary to the region of the template being sequenced, there is no free 3′-OH group available to direct further sequence extension and therefore the polymerase cannot add further nucleotides. Once the identity of the base incorporated into the growing chain has been determined, the 3′ reversible terminator may be removed to allow addition of the next successive nucleotide. By ordering the products derived using these modified nucleotides it is possible to deduce the DNA sequence of the oligonucleotide target nucleic acid sequence.

In embodiments, the methods of sequencing a nucleic acid include a extending a polynucleotide by using a polymerase. In embodiments, the polymerase is a DNA polymerase.

In embodiments, sequencing includes a plurality of sequencing cycles. In embodiments, sequencing includes 20 to 100 sequencing cycles. In embodiments, sequencing includes 50 to 100 sequencing cycles. In embodiments, sequencing includes 50 to 300 sequencing cycles. In embodiments, sequencing includes 50 to 150 sequencing cycles. In embodiments, sequencing includes at least 10, 20, 30 40, or 50 sequencing cycles. In embodiments, sequencing includes at least 10 sequencing cycles. In embodiments, sequencing includes 10 to 20 sequencing cycles. In embodiments, sequencing includes 10, 11, 12, 13, 14, or 15 sequencing cycles. In embodiments, sequencing includes (a) extending a sequencing primer by incorporating a labeled nucleotide, or labeled nucleotide analogue and (b) detecting the label to generate a signal for each incorporated nucleotide or nucleotide analogue.

In embodiments, sequencing includes sequentially extending a plurality of sequencing primers (e.g., sequencing a first region of a target nucleic acid followed by sequencing a second region of a target nucleic acid, followed by sequencing N regions, where N is the number of sequencing primers in the known sequencing primer set). In embodiments, sequencing includes generating a plurality of sequencing reads.

In embodiments, sequencing includes extending a sequencing primer to generate a sequencing read. In embodiments, sequencing includes extending a sequencing primer by incorporating a labeled nucleotide, or labeled nucleotide analogue and detecting the label to generate a signal for each incorporated nucleotide or nucleotide analogue. In embodiments, the labeled nucleotide or labeled nucleotide analogue further includes a reversible terminator moiety.

In embodiments, sequencing includes encoding the sequencing read into a codeword. Useful encoding schemes include those developed for telecommunications, coding theory and information theory such as those set forth in Hamming, Coding and Information Theory, 2^{nd} Ed. Prentice Hall, Englewood Cliffs, N.J. (1986) and Moon T K. Error Correction Coding: Mathematical Methods and Algorithms. ed. 1st Wiley: 2005, each of which are incorporated herein by reference. A useful encoding scheme uses a Hamming code. A Hamming code can provide for signal (and therefore sequencing and barcode) distinction. In this scheme, signal states detected from a series of nucleotide incorporation and detection events (i.e., while sequencing the oligonucleotide barcode) can be represented as a series of the digits to form a codeword, the codeword having a length equivalent to the number incorporation/detection events. The digits can be binary (e.g. having a value of 1 for presence of signal and a value of 0 for absence of the signal) or digits can have a higher radix (e.g., a ternary digit having a value of 1 for fluorescence at a first wavelength, a value of 2 for fluorescence at a second wavelength, and a value of 0 for no fluorescence at those wavelengths, etc.). Barcode discrimination capabilities are provided when codewords can be quantified via Hamming distances between two codewords (i.e., barcode 1 having codeword 1, and barcode 2 having codeword 2, etc.).

In embodiments, the barcode is at least 5, 6, 7, 8, 9, 10, 11, 12, 13, 14, or 15 nucleotides in length. In embodiments, the barcode is 5, 6, 7, 8, 9, 10, 11, 12, 13, 14, or 15 nucleotides in length. In embodiments, the barcode is 10 to 15 nucleotides in length. An oligonucleotide barcode is at least about 1, 2, 3, 4, 5, 6, 7, 8, 9, 10, 15, 20, 25, 30, 35, 40, 45, 50 or more nucleotides in length. An oligonucleotide barcode can be at most about 300, 200, 100, 90, 80, 70, 60, 50, 40, 30, 20, 15, 12, 10, 9, 8, 7, 6, 5, 4 or fewer or more nucleotides in length. In embodiments, an oligonucleotide barcode includes between about 5 to about 8, about 5 to about 10, about 5 to about 15, about 5 to about 20, about 10 to about 150 nucleotides. In embodiments, an oligonucleotide barcode includes between 5 to 8, 5 to 10, 5 to 15, 5 to 20, 10 to 150 nucleotides. In embodiments, an oligonucleotide barcode is less than 10 nucleotides. In embodiments, an oligonucleotide barcode is about 10 nucleotides. In embodiments, an oligonucleotide barcode is 10 nucleotides. An oligonucleotide barcode may include a unique sequence (e.g., a barcode sequence) that gives the oligonucleotide barcode its identifying functionality. The unique sequence may be random or non-random. Attachment of the barcode sequence to a nucleic acid of interest (i.e., the target) may associate the barcode sequence with the nucleic acid of interest. The barcode may then be used to identify the nucleic acid of interest during sequencing, even when other nucleic acids of interest (e.g., comprising different oligonucleotide barcodes) are present. In embodiments, the oligonucleotide barcode consists only of a unique barcode sequence. In embodiments, the 5′ end of a barcoded oligonucleotide is phosphorylated. In embodiments, the oligonucleotide barcode is known (i.e., the nucleic sequence is known before sequencing) and is sorted into a basis-set according to their Hamming distance. Oligonucleotide barcodes can be associated with a target of interest by knowing, a priori, the target of interest, such as a gene or protein. In embodiments, the oligonucleotide barcodes further include one or more sequences capable of specifically binding a gene or nucleic acid sequence of interest. For example, in embodiments, the oligonucleotide barcode include a sequence capable of hybridizing to mRNA, e.g., one containing a poly-T sequence (e.g., having several T's in a row, e.g., 4, 5, 6, 7, 8, or more T's). In embodiments, the padlock probe is at least about 50, 60, 70, 80, 90, 100, 110, 120, 130 or more nucleotides in length. In embodiments, the padlock probe is at most about 300, 200, 100, 90, 80, or fewer or more nucleotides in length. In embodiments, the total length of the padlock probe is about 80, 90, 100, 110, 120, 130, or 140 nucleotides in length.

In embodiments, the oligonucleotide barcode is included as part of an oligonucleotide of longer sequence length, such as a primer or a random sequence (e.g., a random N-mer). In embodiments, the oligonucleotide barcode contains random sequences to increase the mass or size of the oligonucleotide tag. The random sequence can be of any suitable length, and there may be one or more than one present. As non-limiting examples, the random sequence may have a length of 10 to 40, 10 to 30, 10 to 20, 25 to 50, 15 to 40, 15 to 30, 20 to 50, 20 to 40, or 20 to 30 nucleotides.

In embodiments, demultiplexing the multiplexed signal includes a linear decomposition of the multiplexed signal. Any of a variety of techniques may be employed for decomposition of the multiplexed signal. Examples include, but are not limited to, Zimmerman et al. Chapter 5: Clearing Up the Signal: Spectral Imaging and Linear Unmixing in Fluorescence Microscopy; Confocal Microscopy: Methods and Protocols, Methods in Molecular Biology, vol. 1075 (2014); Shirawaka H. et al.; Biophysical Journal Volume 86, Issue 3, March 2004, Pages 1739-1752; and S. Schlachter, et al, Opt. Express 17, 22747-22760 (2009); the content of each of which is incorporated herein by reference in its entirety. In embodiments, multiplexed signal includes overlap of a first signal and a second signal and is computationally resolved, for example, by imaging software.

In embodiments, the method further includes measuring an amount of one or more of the targets by counting the one or more associated barcodes. In embodiments, the method further includes counting the one or more associated barcodes in an optically resolved volume.

In embodiments, the method further includes an additional imaging modality, immunofluorescence (IF), or immunohistochemistry modality (e.g., immunostaining). In embodiments, the method includes ER staining (e.g., contacting the cell with a cell-permeable dye which localizes to the endoplasmic reticula), Golgi staining (e.g., contacting the cell with a cell-permeable dye which localizes to the Golgi), F-actin staining (e.g., contacting the cell with a phalloidin-conjugated dye that binds to actin filaments), lysosomal staining (e.g., contacting the cell with a cell-permeable dye that accumulates in the lysosome via the lysosome pH gradient), mitochondrial staining (e.g., contacting the cell with a cell-permeable dye which localizes to the mitochondria), nucleolar staining, or plasma membrane staining. For example, the method includes live cell imaging (e.g., obtaining images of the cell) prior to or during fixing, immobilizing, and permeabilizing the cell. Immunohistochemistry (IHC) is a powerful technique that exploits the specific binding between an antibody and antigen to detect and localize specific antigens in cells and tissue, commonly detected and examined with the light microscope. Known IHC modalities may be used, such as the protocols described in Magaki, S., Hojat, S. A., Wei, B., So, A., & Yong, W. H. (2019). Methods in molecular biology (Clifton, N.J.), 1897, 289-298, which is incorporated herein by reference. In embodiments, the additional imaging modality includes bright field microscopy, phase contrast microscopy, Nomarski differential-interference-contrast microscopy, or dark field microscopy. In embodiments, the method further includes determining the cell morphology (e.g., the cell boundary or cell shape) using known methods in the art. For example, to determining the cell boundary includes comparing the pixel values of an image to a single intensity threshold, which may be determined quickly using histogram-based approaches as described in Carpenter, A. et al Genome Biology 7, R100 (2006) and Arce, S., Sci Rep 3, 2266 (2013)).

In embodiments, the cell or tissue is stained (e.g., contacted and/or incubated with a stain agent). Non-limiting examples of stains include histological stains (e.g., hematoxylin and/or eosin) and immunological stains (e.g., fluorescent stains). A stain is a chemical agent used to selectively color components of biological tissues or cells to enhance their visibility under a microscope. Stains typically bind to specific cellular structures or organelles, such as proteins, nucleic acids, lipids, or carbohydrates, allowing for the differentiation and identification of these structures. In embodiments, the stain is a fluorescent stain (e.g., an intrinsic stain). Intrinsic or fluorescent stains are chemical compounds that possess the inherent ability to emit fluorescence when exposed to specific wavelengths of light, thereby enabling the visualization of biological structures without the need for additional staining agents; examples include eosin, which absorbs light in the blue-green part of the spectrum (around 490-520 nm) and emits light in the green-yellow part of the spectrum (around 520-550 nm), and Hoechst stains, which bind to DNA and emit blue fluorescence around 461 nm. In embodiments, detecting includes directing an excitation light to the cell or tissue and detecting an emission light from the first fluorescent dye. In embodiments, detecting includes directing an excitation light to the cell or tissue and detecting an emission light from the second fluorescent dye. In embodiments, detecting includes directing an excitation light to the cell or tissue and detecting an emission light from the stain. In embodiments, detecting includes directing an excitation light to the cell or tissue and detecting an emission light from the first fluorescent dye, the second fluorescent dye, and the stain. In embodiments, a cell or tissue can be imaged. In embodiments, the method includes detecting an organelle.

In aspects and embodiments described herein, the methods are useful in the field of predictive medicine in which diagnostic assays, prognostic assays, pharmacogenomics, and monitoring clinical trials are used for prognostic (i.e., predictive) purposes to thereby treat an individual prophylactically. Accordingly, in embodiments the methods of diagnosing and/or prognosing one or more diseases and/or disorders using one or more of expression profiling methods described herein are provided.

In an aspect is provided a method of detecting a disorder (e.g., cancer) or a disease-causing mutation or allele in a cell. In embodiments, the cell includes an oncogene (e.g., HER2, BRAF, EGFR, KRAS) and utilizing the methods described herein the oncogene is identified, thereby detecting a disorder when the presence of the oncogene is identified. In embodiments, the sample includes a nucleic acid molecule which includes a disease-causing mutation or allele. In embodiments, the method includes hybridizing an oligonucleotide primer which is correlated with the disease-causing mutation or allele. In embodiments, the method includes ligating a mutation-specific oligonucleotide primer only when the disease-causing mutation or allele is present in the nucleic acid target. In embodiments, the disease-causing mutation or allele is a base substitution, an insertion mutation, a deletion mutation, a gene amplification, a gene deletion, a gene fusion event, or a gene inversion event.

In embodiments, the mutation or allele is associated with an increased predisposition for one or more diseases, disorders, or other phenotypes. In embodiments, the mutation or allele is associated with a decreased predisposition for one or more diseases, disorders, or other phenotypes. For example, some mutations or alleles are associated with a cancer phenotype, such as decreased growth inhibition, evasion of immune detection, or dedifferentiation. Mutations that can be detected using the method provided herein include for example, mutations to BRAF, EGFR, Her2/ERBB2, and other somatic mutations as exemplified by Greenman et al., Nature (2007) 446:153-158, hereby incorporated by reference in its entirety.

In embodiments, the method further includes contacting the cell or tissue with a stain, wherein the stain specifically binds to a biomolecule. A stain is a chemical agent used to selectively color components of biological tissues or cells to enhance their visibility under a microscope. Stains typically bind to specific cellular structures or organelles, such as proteins, nucleic acids, lipids, or carbohydrates, allowing for the differentiation and identification of these structures. In embodiments, the stain is a fluorescent stain (e.g., an intrinsic stain). Intrinsic or fluorescent stains are chemical compounds that possess the inherent ability to emit fluorescence when exposed to specific wavelengths of light, thereby enabling the visualization of biological structures without the need for additional staining agents; examples include eosin, which absorbs light in the blue-green part of the spectrum (around 490-520 nm) and emits light in the green-yellow part of the spectrum (around 520-550 nm), and Hoechst stains, which bind to DNA and emit blue fluorescence around 461 nm. In embodiments, detecting includes directing an excitation light to the cell or tissue and detecting an emission light from the stain.

One aspect of the present disclosure provides a method for identifying a morphological pattern in a biological sample (e.g., tissue). The method includes, at a computing device including one or more processing cores, a memory, and a display, obtaining a discrete attribute value dataset associated with a plurality of indicia corresponding to nucleic acid molecules detected in situ within a tissue section. Each indicium in the plurality of indicia represents a nucleic acid molecule hybridized and amplified within its native location in the tissue, and the plurality of indicia may number at least 25, at least 50, at least 100, at least 150, at least 300, at least 400, or at least 1000. The discrete attribute value dataset includes one or more spatial projections of the tissue sample and further includes one or more two-dimensional images of a first tissue section obtained from the biological sample. Each two-dimensional image may include at least 100,000 pixel values and may optionally include a histological image of the tissue, such as an H&E (H&E) image or suitable alternative (e.g., bright-field microscopy image of the tissue). The discrete attribute value dataset further includes a corresponding plurality of discrete attribute values for each indicium, the values being derived from in situ sequencing or fluorescent detection of the nucleic acid molecules at distinct loci. Each corresponding plurality of discrete attribute values may include at least 500 discrete attribute values per tissue section. The method further includes obtaining a corresponding cluster assignment in a plurality of clusters for each respective indicium in the plurality of indicia of the discrete attribute value dataset. The corresponding cluster assignment is based, at least in part, on the plurality of discrete attribute values of the respective indicium, or on a corresponding plurality of dimension reduction components derived, at least in part, from those values. The method further includes displaying, in a first window on the display, pixel values of all or a portion of a first two-dimensional image of the tissue section. The method further includes overlaying on the first two-dimensional image, and co-aligned with the first two-dimensional image, (i) first indicia for each nucleic acid molecule assigned to a first cluster in the plurality of clusters and (ii) second indicia for each nucleic acid molecule assigned to a second cluster in the plurality of clusters, thereby identifying a morphological pattern of gene expression within the tissue.

### EXAMPLES

### Example 1. Spatial Biology and Inflammatory Bowel Disease

Characterization of the architectural arrangement of cells and structures in tissues is among the most historically important tools in pathology. Over the last two decades, a bevy of molecular methods have become increasingly important for precision diagnostics, yet until recently, it has been necessary to accept the tradeoff that one can either view the structures of samples or query their molecular details, but not both at the same time. This has left a sizable gap in our understanding of many complex diseases, including autoimmunity and cancer, that exist at the interface of these features. A new class of spatial biology technologies that merge morphology and molecular biology are now changing this paradigm. Although promising, the first generation of these tools have limitations that have relegated much of their impact to small studies at the level of anecdotes, rather than large scale clinical research to advance new therapies. Technical constraints on the number of data types that can be interrogated at once, the level of cellular resolution, and sample throughput represent the biggest barriers. To overcome these limitations, we leveraged the unique foundational technology of a multi-omics platform, the G4X, which enables simultaneous transcript counting, protein quantification and the complete sequencing of variable regions of the genome directly in the undisrupted morphological context of a single 5 micron section of standard formalin-fixed tissue. The G4X can process 10 times the tissue area in half the time of existing platforms, entailing a 20× greater overall throughput.

Inflammatory bowel disease (IBD) is a chronic autoimmune disorder, with ulcerative colitis (UC) and Crohn's disease (CD) representing the main IBD subtypes. Each is characterized by distinct patterns of chronic GI tract inflammation, with UC being primarily restricted to the colon and conferring an increased lifetime risk of colorectal cancer and Crohn's manifesting throughout the gut and frequently complicated by fistulas. Both diseases markedly impact quality of life. Disease flairs and resulting hospitalizations for complications, be they infectious or requiring of surgery, are frustratingly unpredictable. IBD represents a major global burden of disease that is rapidly increasing for reasons that are uncertain but likely relate to changes in diet and lifestyle. Since the turn of the century, in industrialized nations the prevalence has nearly doubled. The US has one of the highest rates in the world: more than 0.7% of the population, representing nearly 3 million Americans, are living with the disease. The economic impact is similarly extensive. The annualized cost of IBD in the US now exceeds $8 billion. Disclosed herein are methods for designing and building the transcript and protein portion of an IBD-focused prototype panel, and using this new panel to process a cohort of 160 real-world IBD clinical samples in an effort to predict treatment response to two modern classes of IBD therapies.

During the last decade, as the mainstay of treatment has transitioned from steroids and cytotoxic agents to targeted biologics, there have been improvements in IBD outcomes overall, but at the level of individual patients these remain inconsistent. Agents targeting TNF (i.e. Infliximab), integrin α4β1 (i.e. Vedolizumab) and IL12/23 (i.e. Ustekinumab) bring remarkable relief to some, but for others the responses are poor or only transient. This high degree of clinical variability reflects the substantial heterogeneity of the underlying molecular pathogenesis of what we currently categorize into two syndromically-defined diseases. While there have been some successes developing predictive blood and tissue-based biomarkers, their performance is modest and clinical adoption has been limited.

Part of the challenge is that IBD is not the result of a single molecule or cell type. The disease results from the interplay between multiple cellular and cytokine aspects of the immune system in the context of higher order immunological and barrier structures of the gut and its associated microbiota. Conventional measurement techniques are inherently blind to many key features of IBD pathogenesis insofar as they measure bulk-extracted populations of molecules (traditional molecular biology) or a handful of targets in the context of morphology (traditional immunohistochemistry). Emerging spatial biology tools that can assess highly multiplexed molecular signals in the context of single cells or native architecture have shown promise for disentangling this complexity but remain exploratory and have important limitations.

IBD is not static over time and varies through the GI tract. Although extensive endoscopic sampling (both spatially and across time) is part of routine clinical care, the low throughput (thus high cost) nature of existing platforms has made them simply impractical for use in large, well powered IBD clinical studies. This challenge has been compounded by the fact that most existing platforms track only one type of analyte (i.e. protein or transcript, thus increasing cost and sample requirements for being able to investigate both) and are not easily adapted to modest plexicity disease-focused applications. Users are often stuck using large, costly panels where most targets are irrelevant to the use-case. The high cost, low throughput, inflexible and awkward multi-platform nature of current spatial multi-omic technologies represent major barriers to the large scale translational studies needed to advance therapy development and patient care in IBD.

The G4X platform is a spatial multi-omics device. The rapid 4-color dye chemistry, fast scanning optics, powerful image processing hardware and multi-flow cell format underlies the exceptionally large high-resolution spatial scanning field leveraged by the G4X. With a 2× faster run time and a 10× greater sample area, the G4X enables a 20× greater throughput than the current spatial platform market leader. Furthermore, this high run capacity is paired with an innovative front-end sample preparation protocol, facilitating a streamlined end-to-end workflow. A unique hydrogel intermediate transfer process allows 5 μm sections of routine formalin-fixed paraffin embedded (FFPE) samples to be easily picked up, then regions of interest punched out into tiles, prior to transfer to an arrayed template, and then onto the flow cell surface followed by lid assembly. Together with the flexibility to use different sized pieces of tissues on each of the four independent flow cells of a G4X run, as part of a 3 day workflow, a single operator can comfortably prepare up to 128 samples per 24 hour run to achieve clinical study-level sample throughput.

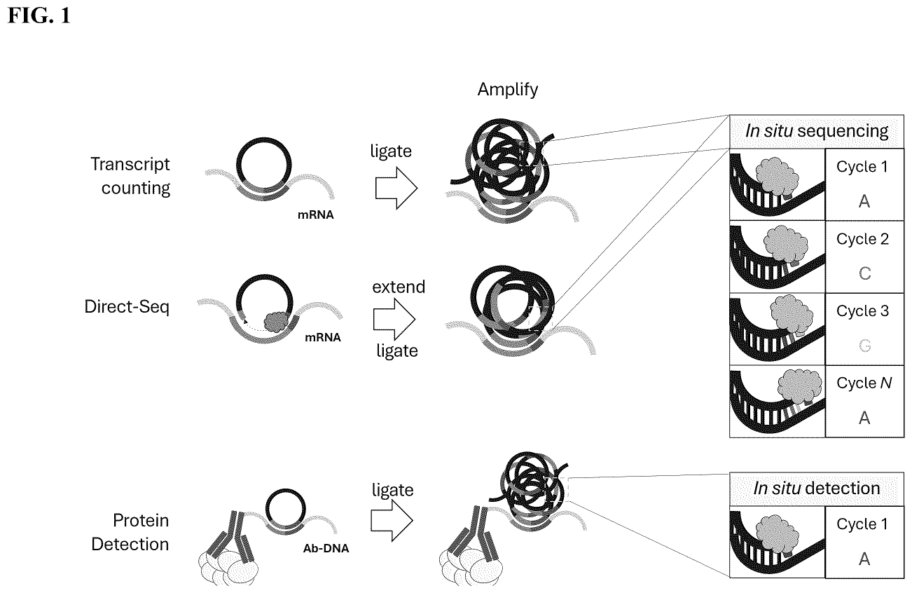

The unique multiomic readout is achieved with fluorescent detection, for example commonly used in sequencing-by-synthesis (SBS) chemistry. Prior to a high-resolution fluorescent nuclear stain (e.g., hematoxylin equivalent) and a cytoplasm stain (eosin), collectively referred to as a fluorescent HE, or fH&E scan, molecular counting of RNA transcripts and proteins begins using a targeted panel of padlock probes against the transcripts or oligonucleotide-linked antibodies. After binding-mediated ligation and rolling circle amplification, SBS readout is completed (see FIG. 1) The high affinity, short binding arm design of the probes allows high efficiency capture of fragmented transcripts often encountered with archival FFPE clinical samples. Nucleotide-level genotyping via Direct-Seq is achieved similarly to transcript counting, but with a gap-fill step prior to probe circulation to incorporate the target sequence into the probe, which may then be detected.

An advantage of G4X is that it is a true multi-omic platform that produces four independent data streams from the same exact tissue section: fluorescent H&E, transcript count, protein expression, and direct sequencing. Not only does this translate to a marked reduction in cost and labor compared with use of adjacent FFPE sections on multiple different platforms, it conserves precious clinical research samples and makes it possible to fully relate together independent types of data types down to the level of individual cells within their native architecture. This degree of resolution is an indispensable feature for characterizing the exceptionally complex host-immune cell and molecular interactions within IBD. We have selected inflammatory bowel disease, a high-prevalence, high-morbidity and high-cost clinical condition to develop and deploy a disease-focused spatial multi-omic panel.

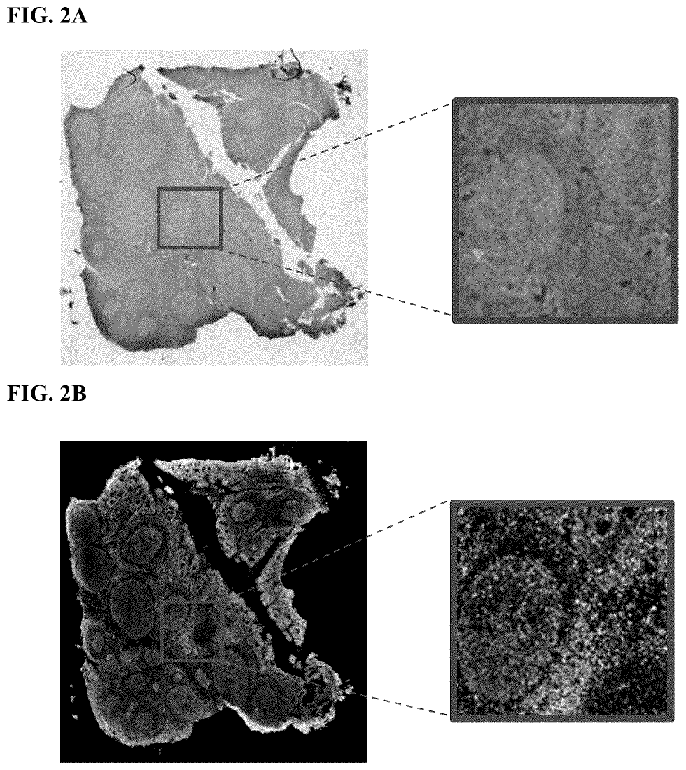

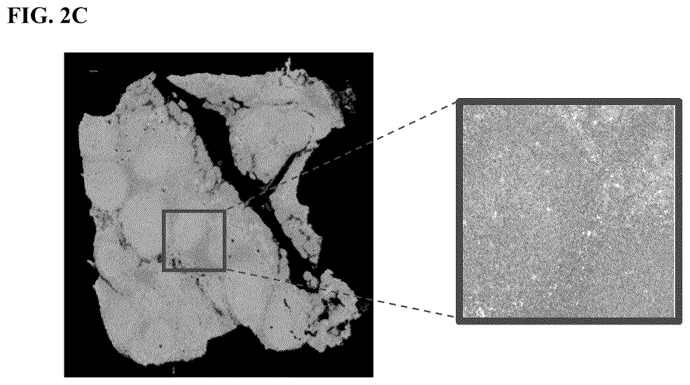

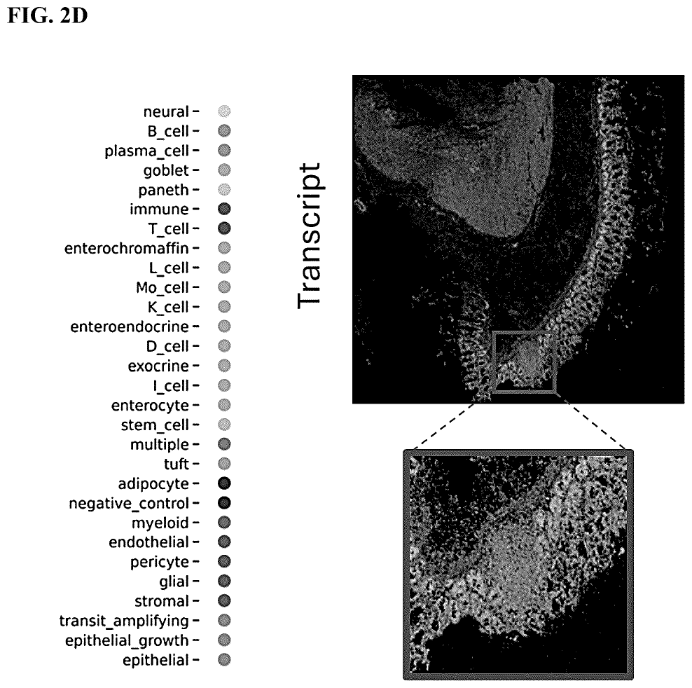

To demonstrate the multi-modal G4X data output, we initially used an immune-focused panel targeting 153 genes and 10 protein targets to highlight intricate lymphoid structures in normal human tonsil (FIG. 2A-C). High resolution UMAPs were assembled from transcript only, protein only and a combined readouts which identified clusters that were distinct from either modality alone. To demonstrate compatibility of the G4X with colon, we used a 155-gene transcript panel on a normal human colon sample (FIG. 2D). As shown at two magnifications, single-cell level resolution from nearly three dozen cell-types was easily appreciable. The data indicates that the G4X spatial biology platform produces single-cell resolution transcript data from colon.

Building a Panel that is Tailored to the Pathophysiology and Therapeutically-Relevant Pathways of IBD.

In one prophetic example, ten banked FFPE (formalin-fixed, paraffin-embedded) ulcerative colitis (UC) samples are selected. These samples are collected endoscopically as part of routine care and processed using standard clinical pathology procedures. The samples are randomly selected, ensuring a fair representation of an expected range of UC disease activity, with the goal that half of the samples are more than five years old. These samples were prepared and analyzed on the G4X system, employing an immuno-oncology (IO) panel comprising approximately 200 genes and 8 proteins. The data generated was processed using the onboard image processing workflow, which includes image registration, cluster detection/base-calling, image stitching, nuclear-based cell segmentation, and output file generation.

For each sample, we can assess the data quality and yield from the each of the 3 information streams for comparison to those from the same panel previously run on normal colon and other tissues. Success of this aim will entail seeing similar average performance (and no drastic increase in outliers) between UC and our historical controls. We can quantitatively assess the following metrics and expect roughly the performance listed per data modality. For the RNA channel, we can enumerate mapped transcripts per cell. We expect ˜50 transcripts per cell, ˜25 unique genes per cell, and a false discovery rate of less than 2% (as determined by both negative control probes and negative control “codewords”). For the protein channel, we can quantify signal-to-noise ratio (protein intensity in positive cells vs negative cells) and expect this to average of ≥5 and with a correlation between protein intensity and paired transcript target expression level of r2≥0.6 per protein, as was done with transcript-based hierarchical cell-type specific clustering in the colon data in FIGS. 4A-C.

For the fluorescent H&E (fH&E™) modality, feedback is solicited from a trained pathologist at the collaborating academic site providing the samples. The pathologist compares the G4X-generated fH&E with a traditional H&E section from each block. Moreover, Hover-Net is employed to classify cell types from the fH&E alone, and these classifications are compared to cell types determined by the RNA modality, with an expected agreement of ≥90% between the two classifications. If more than 20% of the samples fall below the quantitative or qualitative target quality threshold for any given modality, an investigation is conducted to determine if the issue can be attributed to the age of the block, block-to-block variability, or variability in the assay/platform. Additional sections or samples are run as needed to address these issues.

Adding and/or removing target genes or proteins to the panel may be required. This will allow for the rapid identification of any key IBD targets that may present challenges, such as poorly specific or incompatible antibodies, or low transcript copy numbers. The panel is designed to broadly address the following areas: 1) normal structures and cell types of the colon and adjacent tissues; 2) key cell subsets of the adaptive and innate immune systems; 3) components of activity-reflecting signaling pathways implicated in ulcerative colitis (UC) and Crohn's disease; 4) therapy-relevant pathways, including the targets and downstream elements of major and emerging classes of biologic agents; and 5) key transcripts or proteins associated with IBD-related complications, such as neoplastic transformation, fistula formation, and infection. The panel builds upon the contents of prior exploratory immuno-oncology and gastrointestinal (GI) system panels, supplemented with an extensive literature search, preliminary IBD-specific transcriptional profiling data (bulk, single-cell, and lower-resolution spatial technologies), a review by clinical collaborators of ongoing UC and Crohn's disease studies in ClinicalTrials.gov, and interviews with at least five key-opinion-leader experts.

For the protein aspect of the panel, 2-4 additional IBD-relevant proteins are incorporated, focusing on those with expected unique clinical relevance related to targets of IBD therapies and/or markers commonly used in immunohistochemistry (IHC) in GI pathology (e.g., PanCK, CD31, aSMA, KI67). Serotype controls are included as quality control (QC) elements. Commercially-available antibodies are ordered, oligo-conjugated, and tested on ten IBD clinical samples (both active and quiescent). These tests are conducted in groups of four, with basic titrations of antigen retrieval conditions, antibody concentration, and amplification conditions.

For the transcript portion of the panel, approximately 6-12 probes per gene target are designed for each gene, with careful consideration of sequence context, GC content, and splice variants during probe footprint selection. The panel is tested on the same ten samples mentioned above, with 1-2 rounds of relative concentration adjustment or probe dropout conducted to achieve maximally uniform capture without the need for redesign.

Additionally, probes enabling gap-filling (i.e., Direct-Seq™) targeting of variable regions of TCRs, HLA or SNP loci sites associated with disease risk/response to therapy, or hotspots in cancer driver genes are contemplated herein.

In another prophetic example, the IBD panel is used to generate data from 160 UC samples, collected from 80 patients at two time points-before and 14 weeks after the initiation of one of two novel therapeutic agents. This cohort is selected to represent a range of clinical responses to these treatments. The primary objective is to assess the performance of the prototype panel across a broad diversity of real-world samples, gaining insights into the uniformity of target performance and the relevance of each element in the panel. The sample set includes variables that provide hypothesis-generating observations, which inform future panel refinements and support the design of larger clinical utility studies.

The 80 UC cases are selected from a cohort of previously banked, endoscopically-collected FFPE samples, with each case including samples taken before treatment and 14 weeks after treatment initiation. For 40 patients, the treatment involves an integrin α4β1 inhibitor, while the remaining 40 receive an IL12/23 blocker. Within each therapeutic group, 20 patients are identified as good responders (as defined clinically and endoscopically at 14 weeks) and 20 as primary poor responders. This study therefore encompasses 160 real-world IBD samples from patients undergoing modern therapies with clinically-relevant outcomes.

To ensure proper sample preparation and adherence to clinical sample use restrictions, tissue blocks are sectioned and mounted on G4X flow cells at the hospital site of a clinical collaborator. The 160 samples are de-identified and blinded regarding clinical status. A 32-sample/4×4 mm section flow cell format, is utilized with extra sections included as backups. The samples are processed using the IBD G4X panel, as developed previously, with all samples run and data processed uniformly.

Secondary and tertiary analyses include applying a custom multi-modal graph-based clustering method (from the Leiden and igraph libraries) to identify cell types, and a multi-modal dimensional reduction method (from the RAPIDS library) to process the data. These analyses are used to confirm the recovery of expected cell types and expression profiles, and to serve as a basis for cellular neighborhood analysis, aiming to identify therapeutically-relevant cell populations and interactions.

Performance is measured by the quality of the data and the ability to process it in 2-3 runs over the course of 1-2 weeks. The generated data is analyzed for spatial multi-omic correlations regarding: 1) pre- vs post-treatment time points, 2) class of drug, 3) responder vs non-responder status within each modality, 4) relationships between molecular and histological features such as ulceration, 5) cell-cell neighbor interactions predictive of any of the above, and 6) other patient demographics such as age, age-of-onset, prior treatment history, or longer-term outcomes, where available.

Sample availability, human subjects and diversity. In terms of diversity of clinical demographics, the gender, ethnic, and age breakdown of the study population is expected to approximate the broader IBD population for which the future clinical research product will be used. It is well established that the incidence of IBD is as much as 3× higher in the US and Western Europe than elsewhere in the world. The prevalence of UC is 1.5 higher in males than females and has an age peak of 40-50 years old.

The aforementioned products will be important for researchers studying tumor heterogeneity to obtain a more comprehensive view of the tumor microenvironment in the bone marrow by obtaining direct data on tissue morphology, gene expression, protein and specific TCR sequences all within the same tissue section. Furthermore, the ability to sequence critical portions of T-cell receptors in a spatial context to study cell-to-cell interactions and modulations of cellular function will be valuable in directly understanding immune response.

Uncovering clonality data on T-cells will allow better understanding of the phenotypic and function characteristics of the expanded clones to illuminate potential targets for therapies, such as immune checkpoints or other molecular targets to enhance T cell killing of cancer cells. Naturally, one of the interesting aspects of spatial analysis is the ability to also look at a variety of cell types. So, having identified T cell clonotypes in situ, neighborhood analysis could also be evaluated for evidence of cell-cell interaction with not only other T cells, but also other immune cell types such as myeloid and stromal cells. Finally, the same approaches we will utilize for TCR detection could be adapted to BCR detection as well, so that studies of B cell clonality could be undertaken as well. The methods, compositions, and devices described herein can detect gene expression and proteins together with fluorescent H&E all from the same FFPE tissue section. Additionally, the TCR immune profiling panel will enable detection of the CDR3 region of TCR in healthy and diseased FFPE tissue samples on the same platform. As a high throughput assay that can return data on the location of different biomolecules and their proximity to neighboring molecules, these methods will also enable the building of “cellular atlases”. These spatial maps can describe the what, where, and when of different biological processes to drive our understanding of how cells in the human body work together in health, cancer, and treatment.

### Example 2. Profiling of TCRs In Situ

Biology is fundamentally rooted in the cellular structure of tissues. Many current diagnostic and research techniques for examining molecular biology inherently disrupt much of this important architecture. Single-cell sequencing has revealed intricate cellular molecular states, a significant advance over bulk sequencing methods that only offer aggregated information. This technology has dramatically improved our understanding of cancer biology, from elucidating cell-to-cell differences inter- and intratumorally, to enabling tracking of tumor evolution and responses to treatment. The recent emergence of Spatial Multi-Omics technologies promises to provide spatial context to tissue analysis. Currently available spatial platforms fall into two categories: digital spatial profiling (e.g. Visium and GeoMx), which lack single cell resolution, and in situ methods (e.g. Xenium, CosMx and MerScope) which lack the ability to read out unknown sequences. In addition to these limitations, the analysis throughput is limited to a handful of small samples for all these platforms.

We have recently developed methods to perform Next Generation Sequencing (NGS) inside cells and tissues, providing a universal in situ readout for gene transcription, protein expression and direct sequencing of variable regions. As described supra, the G4X spatial sequencer enables unparalleled sample throughput (20-fold greater than any existing platforms) and combined readout of transcriptomics, proteomics and direct sequencing of target sequences (Direct-Seq) in the same FFPE section. Indeed, direct T-cell receptor sequencing as an additional readout will enable the spatial tracking of T-cell clonotypes with paired transcript and protein profiling for clinical-scale studies, which has thus far been unattainable.

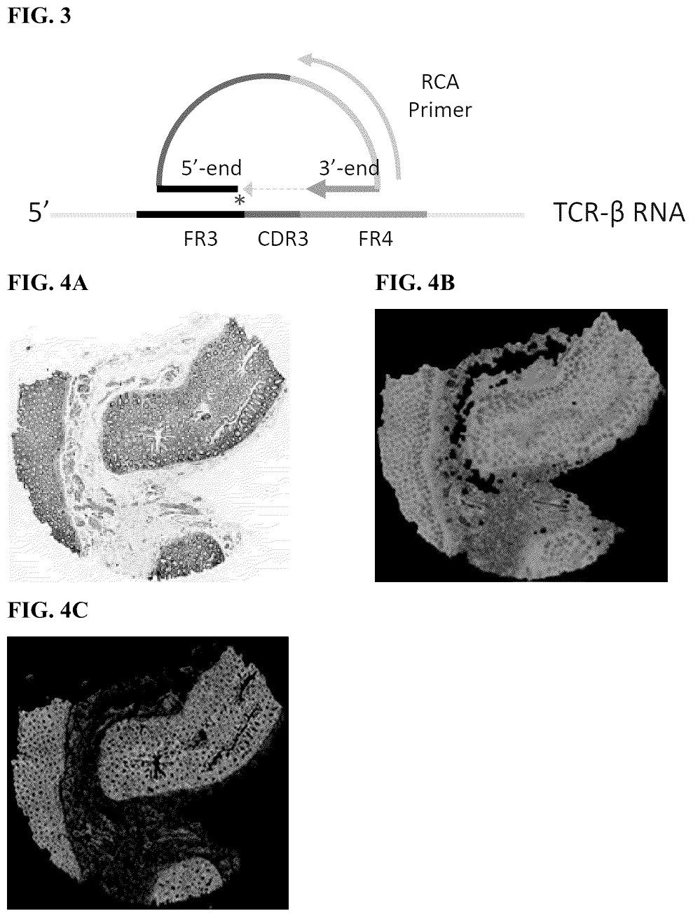

Unified readout for multi-omic profiling in the same section. The unified sequencing readout of all three modalities illustrated in FIG. 1 allows for fast cycle times. In addition, we use fluorescent dyes for the cytoplasm and nucleus to generate fluorescent H&E images (fH&E). We have profiled 105 transcript targets, 3 protein targets and generated a fluorescent H&E image from the same kidney section (see FIG. 3). We chose kidney as an example because of its clear spatial structures easily distinguished by transcript profiling, protein profiling and fH&E, thus allowing easy visual inspection of correspondence of targets and specificity of targeting. The transcript and protein maps have been coded such that targets for the same tissue structure are colored similarly. Example structures that are easy to pick out in multiple modalities are the glomeruli (magenta for transcripts and protein, balls of nuclei in the H&E) and blood vessels (purple in the transcript map and morphologically distinct in the H+E). This data shows that we can profile transcripts, protein, and fH+E in the same tissue section.

Low quality samples: To get sufficient signal for sequencing requires amplification of our targets. As outlined above and illustrated in FIG. 1, for all 3 modalities readout by SBS, one amplification protocol envisioned is rolling circle amplification (RCA). A single probe is sufficient to generate a detectable sequencing result. In the case of damaged samples, this means we only need short intact regions of RNA molecules for detection. The kidney data in FIG. 3 demonstrates that the approach is compatible with FFPE tissue samples and allows working with even more highly degraded samples.

We developed and implemented a custom image processing and bioinformatics pipeline that can be run on sequencing instruments to process the large-scale multi-omic data produced from each in situ sequencing run. First, all acquired images are registered using the x-y locations of fiducial beads as stationary, ground-truth anchors. Then, for transcriptomic and direct-sequencing modalities, spots are detected by fitting a range of Laplacian of Gaussian filters to each fluorescence channel individually, identifying the locations of local peaks, and extracting intensities across all channels and sequencing cycles using a kernel centered on the identified peak. In this way, each detected spot is reduced to a channel & cycle intensity vector, which is then input to a base-caller to produce sequences for each spot. For the transcriptomic panels, these sequences are matched to the expected sequences for each target with a maximum allowed number of errors based on the panel design, while for direct-sequencing applications these sequences are used as-is for downstream analyses. For the protein readout modality, registered images are assembled into tissue section-level montages for each protein; image transformations are applied to correct for the known spectral cross-talk of the fluorescent dyes used, as well as to remove autofluorescence and fiducial beads. Fluorescent H&E conversion is also applied to registered, section-level montages of the corresponding fluorophore channels. Finally, segmentation is performed using a custom-trained cytoplasmic model implemented with Cellpose, which takes as input the protein-based segmentation markers in one channel and a nuclear fluorescent marker in a second channel. Single-cell profiles are constructed by intersecting the transcript/direct-sequencing locations with the segmentation mask and calculating the intensity of each protein within the nuclear and membrane regions of the segmentation mask. The final outputs of the pipeline consist of tissue section-level fluorescent H&E, protein, and segmentation mask images (in OME-Zarr format), transcript/direct-sequencing tables (in csv format), cell by transcript/direct-sequencing/protein tables (in csv format), and a compiled SpatialData object where the coordinates for all outputs are defined by a tissue section-level registered framework.

We have additionally developed a padlock probe designer for our targeted panels that can generate probe sets for any combination of human gene targets with a pre-specified hamming distance criterion and uses the estimated melting temperatures of off- and on-target binding events to maximize specificity.

Optimization of Direct-seq for TCR sequencing in cells. The T-cell receptor (TCR), found on the surface of T cells, plays a crucial role in recognizing antigens and triggering an immune response. Profiling the TCR repertoire reveals the T-cell clonal dynamics that underlie an immune response and may shed light on the antigenic stimuli for such responses. However, analyzing the diverse TCR repertoire is challenging. The small set of genes that encode the T-cell receptor can create between 10¹⁵ and 10²⁰ TCR clonotypes (a clonotype is a population of T cells that carry an identical TCR, see Nikolich-Zugich et al., 2004. The many important facets of T-cell repertoire diversity. Nat. Rev. Immunol. 4, 123-132; and Miles et al., Bias in the [α][β]T-cell repertoire: implications for disease pathogenesis and vaccination. Immunol. Cell Biol. 89, 375-387). Although the most general approach is to sequence the hypervariable complementarity determining region 3 (CDR3) within the TCRβ chain only, we expand this to its paired strand, TCRα, as well.

Taking TCRβ as an example, our implementation of inverted molecular probes (padlock probes) for Direct-Seq to characterize TCR sequences will utilize work from Montagne et al (TCRβ, from ref Montagne et al., 2020. Ultra-efficient sequencing of T Cell receptor repertoires reveals shared responses in muscle from patients with Myositis. EBioMedicine 59, 102972, which is incorporated herein in its entirety by reference for all purposes). Probes will be designed such that CDR3-targeting primer pairs form the 3′- and 5′-end of the probes, which also contain a common primer binding site for RCA amplification (FIG. 5). These synthetic probes will be pooled, mixed with the corresponding probes for TCRα, and tested in pure and mixed populations of cell lines, as described above. Once probes are annealed to TCRαβ RNAs in fixed cells, the 3′-ends are extended by reverse transcription, generating a copy of the highly variable CDR3 region. By using a Reverse Transcriptase (RT) with limited strand displacement activity (e.g., an ‘RT’ as described herein), we have demonstrated that extension can proceed until the extension product encounters the 5′-end of the padlock probe, effectively halting extension. The elongated 3′-end of the probe can then be enzymatically ligated to its own 5′-end further upstream to form a ssDNA circle, which serves as a template for Rolling Circle Amplification (RCA). Post amplification, RCA products are sequenced in-situ. In parallel, we will harvest RNA from each cell line, perform TCR targeted RT, PCR and Sanger sequencing of the CDR3 regions. This will provide a ground truth for our readout sequences obtained in situ. In addition, including transcript and protein detection will allow us to confirm the cell type associated with each CDR3 sequence, also in situ.

We have established and optimized antigen retrieval conditions without compromising RNA detection efficiency and are compatible with a wide variety of antibodies. For each new protein target we identify a prioritized list of antibody clones with well-characterized specificity from literature and clinical diagnostics, and screen for desired staining morphology using standard immunofluorescence readouts in standard assay conditions. To highlight the success of our approach, we recently identified antibodies for 14 new protein targets by screening 47 unique antibody clones and identifying a top candidate to conjugate for each target. Once antibodies are conjugated to a unique DNA oligo (antibody-oligonucleotide (Ab-O) conjugates), we confirm staining morphology of conjugated antibodies matches that of unconjugated antibodies using fluorescent secondary antibodies and sequencing output.

In-situ measurements of RNA, proteins, and TCR sequencing. We will apply the methods and reagents described herein to profile at least 200 gene transcripts and 20 or more proteins. Additionally, we will also use the in-situ TCR sequencing methods described herein to analyze the highly variable CDR3 regions in the Alpha and Beta receptors.

Once the 5-micron thick tissue sections have been deposited on the functionalized slides (e.g., a solid support as described herein), the tissue sections undergo removal of paraffin and heat-induced breaking of fixation bonds. Then, DNA-labeled antibodies will be incubated with the samples, and padlock probes will be hybridized to the RNA targets. After washing steps, the padlock probes will be ligated to form circular products. For TCR sequencing, an additional step is introduced where the targeted RNA sequence is copied into the padlock probe, and then ligated to the 5′ end of the probe. Based on published articles on B & T cell receptor profiling, it is expected the number of probes to multiplex for Direct-Seq of TCR profiling will be in the order of 400-600 probes. From there, subsequent steps will be performed including amplification (e.g., rolling circle amplification (RCA)), binding of sequencing primers, and 4-color sequencing. Multiple cycles of priming & sequencing will be used to enable higher density readout, by separating different groups of genes & proteins into different sequencing cycles. Each sequencing cycle ends with a permanent termination step (e.g., using irreversibly terminated nucleotides, such as ddNTPs), so that those targets no longer generate any sequencing reads.

The images acquired at each sequencing cycle will be automatically registered (cycle to cycle, and among the 4 color channels) using fluorescent particles as fiducials. The signals from each amplification cluster will be located and extracted to determine the sequence of the RNA targets. The in situ sequence data will be used to create a map of gene expression, with sub-micron positional accuracy. Protein images will be processed and stored in a near-lossless compressed format for further analysis.

Following cell segmentation, based on a combination of nuclear staining and membrane staining, single cells will be identified, and expression of transcripts will be used for clustering and annotation. We also take advantage of the protein measurements to assist with classification of cell type and state. Then, we assess the abundance of each cell type at each time point and treatment condition. We will then leverage the spatial information to computationally deconvolute cell type mixtures in the spatial transcriptomics data to identify niches of different leukemia and/or immune cell composition. We will fully characterize the quantity and composition of leukemia-immune niches in each disease and treatment setting to better understand what cell types interact together in coordinated networks to mediate response or resistance. Selected cell-cell interactions identified may be further validated with proximity ligation assays in additional studies.

TCRα and TCRβ CDR3 sequences will be assigned to segmented CD4+ and CD8⁺ T-cells, and clonotypes will be defined by T-cells that share identical TCRα and TCRβ CDR3 sequences. We will utilize these data to address two major questions: is there evidence of antigen-specific (whether shared across patients or patient-specific) expansion prior to therapy and how does therapy affect clonal evolution both in terms of expansion rates as well as activation status? The first question will be answered by assessing clonality as defined by strict nucleotide sequence similarity as well as by functional convergence, i.e. using the amino acid sequence, secondary structure, and binding characteristics to identify functionally similar CDR3s as described in recent methodologies, and determining the extent of clonotype overlap across patients. (Zhang, Z., Xiong, D., Wang, X. et al. Mapping the functional landscape of T cell receptor repertoires by single-T cell transcriptomics. Nat Methods 18, 92-99 (2021) and Huang, H., Wang, C., Rubelt, F. et al. Analyzing the Mycobacterium tuberculosis immune response by T-cell receptor clustering with GLIPH2 and genome-wide antigen screening. Nat Biotechnol 38, 1194-1202 (2020)) The second question will be answered by tracking clonotype fate for each patient across the therapeutic time course and utilizing the paired transcriptional profiles to determine activation state at each time point. We envision that these data will serve as the basis for follow-up studies where the identity of antigens targeted by expanded and/or shared clonotypes can be elucidated. Additionally, the methodology may be extended to similar in-situ profiling of B-cell receptor sequences, as we have demonstrated in proof-of-principle studies.

In humans, the adaptive immune system's complexity is underscored by the unique mechanism of T-cell receptor (TCR) generation, a process essential for the specific recognition of antigens by T lymphocytes. This process involves the random rearrangement of genomic V(D)J-variable (diversity) joining-segments, mirroring the generation of antibody diversity in B lymphocytes through somatic VDJ recombination of the B-cell receptor locus. The result is a heterodimeric TCR, composed of αβ or γδ chains, each a product of V(D)J recombination. This sophisticated genetic recombination endows T lymphocytes with the capacity to recognize a vast array of antigens, central to the body's adaptive immunity.

The focal point of TCR diversity, the complementarity-determining region 3 (CDR3), encompasses the VDJ recombination junctions and encodes the TCR segment that directly interfaces with peptide-bound major histocompatibility complex molecules. Given its pivotal role in antigen recognition and the consequent immune response, the CDR3 sequence and the identity of the flanking V and J gene segments become invaluable markers for classifying TCR variants. This intricate diversity of TCRs, generated exclusively within T cells, presents a unique opportunity to employ TCR profiling as a precise tool for enumerating and quantifying T-cell clonality, a technique that holds significant implications for the diagnosis and treatment of hematologic malignancies.

The evolution of methodologies for characterizing T-cell proliferation and diversity reflects scientists' growing understanding of the immune system's complexity. Initial techniques, such as Southern blot hybridization, polymerase chain reaction (PCR), and flow cytometry, laid the groundwork for exploring T-cell behavior and clonality. However, the advent of next-generation sequencing (NGS) technologies marked a pivotal shift, enabling an unprecedented depth and breadth of analysis. NGS facilitates extensive, high-throughput measurement and analysis of both TCRs and B-cell receptors (BCRs), offering a window into the immune system's functionality in both health and disease. This technological leap has led to the estimation that a single human individual harbors approximately 106 unique TCR sequences, a testament to the adaptive immune system's versatility.

### Example 3. Code-Probe Colon Panel

The human cell contains about 20,000 to 30,000 genes coding for functional proteins, yet only a small fraction is expressed in any cell at any time. The specific phenotype of a cell is thus derived from the expression of the subset of genes, and studies of cell phenotypes can be performed by gene expression analysis. Messenger RNA (mRNA) is the molecular carrier transporting genetic information from nuclear DNA to the cytoplasmic machinery of protein synthesis. Consequently, mRNA provides an ideal point of investigation in understanding gene functions and correlating their expression to the physiological and pathological phenotype of cells and tissues. For example, analyzing gene expression levels and the biological pathways associated with the genes involved in a cancer, enables one can study the difference between normal cell and cancerous cell pathways to determine the genetic origin of the faulty pathway, thereby identifying potential targets for treating cancer. Furthermore, gene expression analysis facilitates the discovery of biomarkers and gene signatures, which are crucial for diagnosing diseases, monitoring their progression, and predicting responses to therapeutic treatments. In cancer, gene expression profiling aids in developing gene biomarkers and signatures that enhance diagnosis, track disease progression, and predict treatment outcomes, ultimately contributing to personalized medicine approaches.

Traditional methods for analyzing gene expression are essential tools in molecular biology, involving the extraction and quantification of mRNA from cells or tissues. One foundational technique is Northern blotting, where specific RNA molecules are detected within a sample by separating RNA by gel electrophoresis, transferring it to a membrane, and hybridizing it with a labeled complementary probe, with the signal intensity reflecting the presence and quantity of the target RNA. A brute-force approach of capturing gene expression information is simply by isolating regions of interest from within a sample. For example, these regions can then be individually placed in test tubes for RNA extraction and subsequent gene expression profiling. For comprehensive data on the entire transcriptome, RNA sequencing (RNA-Seq) is often employed. In RNA-Seq, RNA is converted to cDNA and used to make a library of nucleic acid molecules, which are then sequenced on next generation sequencing (NGS) platforms like the G4™ or NextSeq 2000™.

Instead of extracting RNA molecules from individual parts (or cells) within a tissue, one can visualize them directly in their original environment. Detecting RNA in situ with spatial surroundings provides critical insights into the spatial organization of gene expression, enhancing our understanding of tissue architecture, cellular interactions, developmental processes, disease mechanisms, and functional genomics. Detecting nucleic acid molecules in cells or tissues enables the observation of spatial heterogeneity within tissues, revealing how gene expression varies across different regions and cell types, and illuminates how local interactions regulate gene expression patterns. Technologically, in situ RNA detection preserves spatial information that is often lost in traditional bulk RNA sequencing, providing a more accurate representation of gene expression patterns. With single-cell resolution, it enables detailed analysis of cellular diversity and heterogeneity, and when combined with other imaging techniques, it offers a multi-dimensional view of cellular function.

An early variant of in situ hybridization (ISH) detection was published in the late 1960s (J. G. Gall, M. L. Pardue, Proc. Natl. Acad. Sci. USA 1969, 63, 378) and has since evolved for visualizing gene expression. One widely used in situ technique is Fluorescence In Situ Hybridization (FISH). FISH utilizes fluorescently labeled probes that hybridize to specific RNA sequences within the tissue. This method allows for the visualization and localization of target RNA molecules with high spatial resolution. FISH is particularly useful for detecting specific gene transcripts and chromosomal abnormalities. However, the number of distinct fluorophores that can be used simultaneously is limited due to spectral overlap. The number of RNA species that can be simultaneously imaged by FISH has been limited. Most experiments stain and image only one RNA species at a time, and even the most advanced multiplexing efforts have only extended this approach to the simultaneous measurement of approximately 10-30 RNA species (Lubeck E, Coskun A F, Zhiyentayev T, Ahmad M, Cai L. Single-cell in situ RNA profiling by sequential hybridization. Nat. Methods. 2014; 11:360-361 and Martin K C, Ephrussi A. mRNA localization: gene expression in the spatial dimension. Cell. 2009; 136:719-730). However, many interesting questions require much higher levels of multiplexing, such as transcriptional definitions of cell phenotyping. Additionally, the resolution of FISH detection, while sufficient for many applications, may not be adequate for detecting very small or low-abundance transcripts, potentially leading to incomplete or biased gene expression data.

Multiplexed, Error-Robust, Fluorescence In Situ Hybridization (MERFISH) (Chen et al. Science. 2015; 348:aaa6090) enables multiplexing by assigning barcodes to different RNA species and then reading out these barcodes through successive rounds of hybridization and imaging on the same sample. NanoString Technologies (now Bruker Spatial Biology) provides a platform for in situ gene expression, the CosMX Spatial Molecular Imager, which utilizes barcoded probes to target thousands of RNA sequences simultaneously, greatly improving multiplexing. 10× Genomics also offers a platform to in situ analysis relying on barcoded probes. In these approaches, each RNA species is often identified not by a single color of fluorescently labeled FISH probe but rather with a unique combination or “barcode” of colors. For example, one RNA molecule might be identified by red-labeled probes alone while another might be identified by the combination of red-, green-, and yellow-labeled probes. The barcoded probes extend the concept used by FISH probes, by replacing the single fluorescent label with a barcode domain. The barcode domain contains consecutive regions that hybridize to unique reporters that are read out over sequential imaging rounds. In practice, a substantial limitation to multiplexing arises from the readout errors that are inherent to FISH. For example, occasionally an RNA molecule that should fluoresce in one imaging round does not accumulate enough fluorescently labeled probe to produce a bright enough signal to be detected. Additionally, stray probes or a bright autofluorescent spots in the cell can sometimes produce a spot bright enough to be called an RNA when it is not actually present.

Barcoded probes typically include both a target sequence binding domain (e.g., 60-80 nucleotides) and a unique barcode domain (e.g., 40-50 nucleotides). The necessity of unique barcodes for each target limits the number of RNA species that can be analyzed simultaneously, constraining multiplexing capacity. Managing these barcodes introduces additional complexity and potential sources of error, as there is a risk of barcode cross-reactivity and misassignment, which can lead to erroneous data. Furthermore, the added barcode sequence may interfere with the binding efficiency or stability of the probe, potentially compromising sensitivity and specificity. For example, if a barcoded probe binds to an off-target sequence due to a mismatch in the target domain or off-target hybridization of the barcode domain, the detection of the barcode could lead to a false positive, mistakenly identifying the target RNA as present. Barcodes can also increase the molecular weight and steric hindrance of probes, potentially affecting their performance in dense or complex tissue environments.

In the context of in situ RNA detection, relying on barcodes for target identification presents several challenges. One significant issue is the limited spatial resolution that can result from insufficient barcode sequence length. If the barcode is too short, it may not generate enough unique sequences to accurately identify and distinguish between different RNA molecules within the complex tissue environment, leading to overestimation of molecular counts due to amplification artifacts. On the other hand, an overly long barcode sequence or an inadequate number of barcode sequences can result in under-sampling, where not all unique RNA molecules are captured, causing underestimation of molecular counts. Additionally, errors during barcode sequence synthesis or sequencing can introduce noise and cause misassignment of barcodes, complicating the spatial mapping of RNA molecules and leading to inaccuracies in gene expression data. This misidentification can be particularly problematic in in situ hybridization, where precise spatial information is crucial for understanding tissue architecture and cellular interactions. Moreover, the additional steps required for decoding and mapping barcode sequences increase the complexity and computational burden of the analysis, making the process more time-consuming and prone to errors.

In contrast, the “barcode-free” probes described herein includes the target sequence binding domain and utilizes sequencing that domain for target identification, offer significant advantages. These probes simplify design and synthesis, reduce production costs, and enhance scalability. Without barcodes, there is less risk of cross-reactivity, and the binding efficiency and stability of the probes are potentially improved, leading to higher sensitivity and specificity. Additionally, without the need for barcode decoding, data processing is streamlined, reducing computational demands. The streamlined data processing reduces computational demands, making “barcode-free” probes a superior choice for comprehensive and efficient in situ gene expression analysis. The uniqueness of these probes enables the detection of a vast array of genes, (e.g., greater than 250 different target sequences) within the same sample without unintended non-specific interactions with other cellular components, particularly genomic DNA which exhibits a natural non-specific affinity for single stranded DNA. Careful design of the sequences is thus required to ensure target specificity and orthogonality to other probes when detecting multiple targets.

The approach described herein significantly enhances specificity and robustness by requiring two hybridization events for detection of a target sequence. Each oligonucleotide probe includes two target hybridization sequences (code-foot 1 and code-foot 2). These sequences bind to adjacent regions of the target RNA. Following successful binding of both code-feet, a ligation step forms a circular polynucleotide, which may be amplified using rolling circle amplification (RCA), and subsequently detected. This dual-binding requirement ensures that if only one code-foot binds to an off-target sequence, the second code-foot will likely fail to bind, preventing the formation of an amplifiable circular probe and thus avoiding false positives. This mechanism significantly enhances the accuracy and specificity of target detection compared to single hybridization events employed in common barcoded probe techniques.

In our current implementation of Code-Probe detection, each oligonucleotide probe includes three basic components. A code probe is a single-stranded oligonucleotide that includes a first code-foot (e.g., 3′ code-foot) and a second code-foot (e.g., 5′ code-foot) on either end and is linked together via a linking oligonucleotide sequence (referred to as a backbone). The linking oligonucleotide sequence may include one or more primer binding sequences (e.g., an amplification and/or sequencing primer binding sequence) and/or an error-correcting barcode which may be sequenced alone and/or in combination with the code-feet.

In designing our panel of probes for gene expression analysis, we imposed several stringent constraints to ensure specificity and efficiency. Each probe consists of two hybridization sequences, referred to herein as code-feet, that are typically less than 20 bp each. Additionally, each code-foot was constrained to a GC content of 45-65% and prohibited from containing four consecutive guanines (Gs) or cytosines (Cs) to minimize the risk of secondary structures and non-specific binding. The binding of the probe to the target sequence should be tight and highly specific in order to avoid any interference, for example having less than or equal to 8 bp overlap with any backbone. The thermodynamics of base pairing, in fact, determine the strength of binding while the nucleotide combination ensures its specificity as a consequence of the uniqueness of base succession. However, unique sequences endowed with high binding energy are not necessarily good probes if the target sequence is inaccessible due to secondary structures.

Both the sensitivity and specificity of a particular probe depends upon the binding energies, AG, of polynucleotide molecules to the probe. In particular, the hybridization intensity traces a sigmoidal curve which follows the melting curve of the probe, decreasing as the binding energy increases. Specificity, however, is maximum at or slightly above the melting temperature of the probe (i.e., at a binding energy that is equal to or slightly greater than zero). Thus, in embodiments, the sensitivity and/or specificity are determined or predicted from the binding energies, provided additional criteria described herein are met. It is noted that the skilled artisan readily appreciates that the term “binding energy,” as used herein, refers to the difference of the energy of polynucleotide molecules (e.g., a target polynucleotide and a polynucleotide probe) when they are in a bound state (i.e., when they are bound or hybridized to each other) from when they are in an unbound state. This definition is readily expressed mathematically by the formula ΔG=G_{bound}−G_{unbound}. Thus, a polynucleotide that has the “largest” binding energy to a particular probe is one for which the difference between the energies of the bound and unbound polynucleotide is greatest. In particular and as the skilled artisan also readily appreciates, because the energy of polynucleotides in a bound state is ordinarily lower than the energy of the unbound polynucleotides, the binding energy (i.e., AG) will ordinarily be a negative number. Thus, as used herein, the polynucleotide having the “largest” binding energy to a particular probe will, in fact, be the polynucleotide for which AG is the most negative.

Reliable oligonucleotide design is crucial for successful binding and detection, and given the diversity of targets and multiplexing, the design of oligonucleotides requires flexibility in the approach. Thus, a number of oligonucleotide design tools exist, for example PrimerSelect (Plasterer T N., Methods Mol. Biol., 1997, vol. 70 (pg. 291-302)), Primer Express (Applied BiosystemsPrimer Express® Software Version 3.0 Getting Started Guide, 2004), OLIGO 7 (Rychlik W., Methods Mol. Biol., 2007, vol. 402 (pg. 35-60)) and Primer3 (Untergasser, A. et al., Nucleic Acids Res 40, e115 (2012)). Additional online tools, for example the OligoAnalyzer™ Tool provided by Integrated DNA Technologies (accessible at www.sg.idtdna.com/pages/tools/oligoanalyzer) or PrimerROC (Johnston, A. D., Lu, J., Ru, Kl. et al. Sci Rep 9, 209 (2019)) sheds additional insight into the secondary structure of oligonucleotides and the resulting amplification products, as well as the self- and heterodimerization tendencies of each primer set. Primer-BLAST is another web service that supports the selection of effective sequences by considering opportunities for mispriming across an entire genome or transcriptome (Sayers E W et al., Nucleic Acids Res., 2012, vol. 40 (pg. D13-D25)). Additionally, RNAFold, provided with ViennaRNA Package 2.0 (Lorenz R, et al. Algorithms Mol Biol. 2011 Nov. 24; 6:26) utilizes thermodynamic principles to determine the most stable structure based on the sequence provided, offering insights into the functional properties and cross-interactions. These tools are useful starting points for evaluating oligonucleotide sequences.

A goal of effective oligonucleotide design is to maximize detection and minimize off-target hybridization, without introducing any biases (e.g., skewing the amplification products to over- or under-represent targets). Primer3 allows for the selection of the binding sequence on the basis of melting temperature (Tm), primer length, and 3′-end stability, which was considered when designing each primer set. Calculating the melting temperature and performing thermodynamic modelling for estimating the propensity of oligonucleotides to hybridize with other oligonucleotides or to hybridize at unintended sites in the genome offer an accurate approach for predicting the energetic stability of DNA structures. For example, because of electronic effects of nucleobase stacking, the stability of 5′-CT-3′ hybridized to 3′-GA-5′ is different from that of 5′-CA-3′ hybridized to 3′-GT-5′, despite the base pairings C:G and T:A are the same. It is recommended to perform oligonucleotides analysis with sophisticated modelling capabilities to capture such electronic effects. Additionally, in silico validation of oligonucleotides may be useful. The online software OligoAnalyzer™ Tool provides information on secondary structure and the possibility of self- or heterodimer formation by the oligonucleotides sequence itself by calculating the Gibbs free energy (AG).

The design further stipulated that the feet of each probe (i.e., the hybridization sequences) must have a maximum overlap of 8 bp with each other and with the probe backbone, ensuring structural stability and minimizing non-specific interactions. Probes were specifically targeted to exonic regions, excluding any intronic sequences to focus on mature mRNA transcripts and avoid potential complications from unspliced pre-mRNA. This approach differs from alternative probes described in the literature and commercially available, which includes intronic sequences in their target binding regions.

Spacing between probe binding sequences is another complicated design consideration. Regular spacing (tiling) is the most common approach because it is easy to implement, but it does not ensure optimal positioning of probes. The geometry of folded conformations of a given RNA around the target sequence can impair the binding of a probe. The folding of RNA is a unimolecular process occurring spontaneously in situ, dependent upon the pairing of self-complementary stretches of different regions of an RNA molecule which produces a number of different secondary structures. Consequently, predictions and experimental validation of secondary structures of RNA are useful for selecting target elements.

To ensure high specificity, we required that the probes exhibit zero off-target hits. Off-target hits were defined using a refined approach: instead of merely counting base pairs matched in each foot, we used the predicted melting temperature (Tm) of the off-target alignment for each foot and enforced a minimum difference between the maximum off-target Tm and the maximum on-target Tm across the two feet for each probe. The true on-target Tm may be higher, but we set a maximum off-target Tm of 40° C. and an on-target Tm of 50° C. to ensure specificity. Using BLAST against the RefSeq RNA database with an E-value cutoff of 100, we refined our off-target definition to include a 3 bp cross-junction requirement, which potentially leads to false-negatives but ensures rigorous specificity. Additional cross checks against GENCODE, RefSeq, or CHESS databases can also be performed. With these parameters, we can generate at least one probe for all genes in our colon panel, optimizing both the specificity and efficiency of our probe design. In embodiments, we generate at least 3 probes per gene. In embodiments, we generate 3, 4, 5, 6, 7, 8, 9, 10, 11, or 12 probes per gene.

Each respective code-foot serves two purposes, binding to a specific target sequence, and following circularization, each code-foot may be detected to provide an identifier for the target sequence. The oligonucleotide probes used herein must satisfy two purposes: (1) specifically bind to a target nucleic acid molecule including a target sequence and (2) maintain dissimilarity between all other binding sequences to uniquely identify the target nucleic acid molecule. Regarding the first goal, designing target binding regions with a high melting temperature (Tm) alone is not sufficient. The geometry of folded RNA conformations around the target sequence can hinder probe binding. RNA folding is a spontaneous, unimolecular process, involving the pairing of self-complementary regions of the RNA molecule, which produces various secondary structures. Therefore, accurate predictions of RNA secondary structures are essential for selecting target sequences that are suitable for probe design. Further, to enable identification of unique targets the resulting sequences should be designed to include orthogonality (e.g., ensuring a particular Hamming distance) to increase the accuracy of target identification. The Hamming Distance between two sequences (i.e., the number of nucleotides that must be switched to convert one barcode sequence into another) is a useful concept in understanding the behaviors of these encoding schemes. Increasing the minimum Hamming Distance between used sequences by leaving more possible sequences unassigned will produce even larger reductions in a misidentification rate

To demonstrate colon specific multi-modal G4X data output, we used a colon-focused panel comprising 155 genes and 8 protein targets to highlight intricate crypt structures in normal human colon (FIGS. 4A-B). Even with this small panel we observed a mean of 99 transcripts and 29 unique genes per cell. We applied supervised hierarchical clustering of transcript-only data to patterns from known cell types. When overlaid as cell-type specific pseudocolors on the fH&E morphology channel, cell type locations relative to crypt structures were as expected (FIG. 4C). When visualized as individual cell type channels, these closely approximated the distribution of several cell-type specific protein channels. Taken together, the data indicates: 1) existing spatial platforms (G4X) can identify regional differences in expression patterns of normal colon vs. UC, 2) the G4X produces single-cell resolution transcript data from colon and, 3) the G4X delivers robust multi-modal data where transcript-based cell typing corresponds as expected to that of morphology and proteins.

We further developed the colon probe panel, designed with stringent constraints to ensure high specificity and efficiency. Each probe consists of two code-feet having a GC content of 45-65% and do not contain four consecutive guanines (Gs) or cytosines (Cs), minimizing secondary structures and non-specific binding. The probes target exonic regions, excluding intronic sequences to focus on mature mRNA transcripts. We ensured zero off-target hits by defining off-targets based on predicted melting temperatures (Tm) and requiring a minimum difference between the maximum off-target Tm and the maximum on-target Tm across the two feet of each probe. See FIG. 3 for an example of the workflow. Using these stringent criteria, we achieved a maximum off-target Tm of 40° C. and an on-target Tm of 50° C., allowing us to generate at least one probe for all genes in our panel. This approach ensures reliable and accurate detection of target gene sequences, crucial for advancing cancer research and therapy. For some genes we purposely designed fewer than 24 probes, this was based on empirical data showing that these genes are extremely abundant in some cell types, leading to optical density issues. For other genes, the natural sequence diversity did not allow us to design 24 probes within our design constraints (e.g., GC content, Hamming Distance). The probes provided herein, for example in the sequence listing and incorporated herein, discloses the 5′ code-foot and the 3′ code-foot useful for targeting particular gene targets: ACKR1, ACKR4, ACSS3, ACTA2, ADIPOQ, ANGPT2, ANKRD29, ANO1, ANXA1, ANXA13, APC, AQP1, AQP3, BEST2, BEST4, BRAF, CA2, CA7, CAMK2N1, CAVIN2, CCK, CCL2, CCL20, CCL3, CCL4, CCL5, CCR2, CCR5, CCR7, CD14, CD163, CD19, CD1C, CD2, CD247, CD27, CD274, CD276, CD28, CD33, CD34, CD36, CD38, CD3D, CD3E, CD4, CD40, CD40LG, CD44, CD47, CD68, CD70, CD74, CD79A, CD80, CD86, CD8A, CDC25C, CDH1, CDH19, CDX2, CEACAM1, CEACAM5, CEACAM8, CENPK, CHGA, CHRM2, CKB, CLCA1, CLCA4, CLDN1, CLDN3, CLDN4, CLEC9A, CLU, CNTNAP2, COL1A1, CPB1, CSF1R, CSPG4, CTLA4, CTNNA2, CTNNB1, CTSS, CX3CL1, CX3CR1, CXCL10, CXCL11, CXCL13, CXCL9, CXCR3, CXCR4, CXCR5, CXCR6, CYP1A1, CYP2A6, DDIT4, DEFA5, DES, DGKG, DPT, DSP, EGFR, ELF3, EOMES, EPCAM, EPHB3, ERBB2, ESM1, FABP1, FAM210B, FAP, FAS, FASN, FBLN1, FBN1, FCAR, FCGR1A, FCGR3A, FGB, FGFR4, FN1, FOXP3, FSCN1, FZD7, GATA3, GATM, GIP, GNLY, GPC1, GPR183, GPRC5A, GPX2, GREM1, GREM2, GRHL1, GUCA2A, GZMA, GZMB, GZMH, GZMK, HAVCR2, HLA-A, HLA-DRA, HNF4A, HOXD8, ICOSLG, ID2, IDO1, IER3, IFNG, IGFBP7, IGHA1, IGHD, IGHG1, IGHM, IL10, IL10RA, IL17A, IL18R1, IL1B, IL2RA, IL2RB, IL6, IL7R, INS, IRF1, ITGAM, ITGAX, ITGB2, ITLN1, JAK3, JCHAIN, KDR, KIT, KLF1, KLRB1, KLRD1, KLRF1, KLRK1, KRAS, KRT20, LAG3, LAMC3, LARS1, LGR5, LPL, LTBP2, LUM, LYVE1, LYZ, MADCAM1, MAPK1, MET, MKI67, MLN, MMP1, MMRN1, MMRN2, MRC1, MS4A1, MUC1, MUC12, MUC2, MUC5B, MUC6, MYC, MYH11, NCAM1, NCR1, NEUROD1, NEUROG3, NKG7, NONO, NOVA1, NRXN1, NTS, ODC1, OLFM4, PAX2, PDCD1, PDCD1LG2, PDE4A, PDGFRA, PDGFRB, PDK4, PECAMI, PHGR1, PIM1, PLAT, PLIN1, PLXND1, POLD2, POLR2A, PON2, POSTN, POU2AF1, POU2F3, PPARG, PRDM1, PRF1, PROM1, PROX1, PTGS2, PYGB, PYY, RARRES1, RBP2, REG1A, REG4, RGMB, RGS5, ROBO1, ROBO2, RRM2, RSPO3, RUNX1, S100A9, S100P, SCGN, SDC1, SELE, SELL, SETD5, SH2D6, SKA3, SLC16A1, SLC2A1, SLC3A2, SLC6A19, SLC7A5, SMOC2, SNCA, SNCG, SOX10, SPINK4, SST, ST14, STAT1, STAT3, STAT4, STC1, SYTL2, TAGLN, TAP1, TAP2, TBX21, TCF7, TCL1A, TFF3, TFP1, TGFB1, THBS1, THY1, TICRR, TIGIT, TLR2, TLR4, TLR9, TNF, TNFRSF17, TNFRSF4, TNFRSF9, TNFSF13B, TNFSF9, TOP2A, TP53, TPH1, TSPAN8, VCAM1, VCAN, VEGFA, VIM, VWF, WARS1, WNT2B, WNT5B, and ZNF800.

In embodiments, the nucleic acid molecule is an RNA molecule and the oligonucleotide probe is designed to bind an exon of the RNA molecule. By targeting exonic sequences, the probes preferentially hybridize to mature, fully processed messenger RNA transcripts rather than to unspliced pre-mRNA or intronic regions. The probes may be configured as dual-hybridization probes, such as code-probes or padlock probes, in which a first hybridization sequence binds to a first exonic region of the transcript and a second hybridization sequence binds to an adjacent exonic region. Upon hybridization of both sequences, the probe may be ligated to form a circular oligonucleotide, which can subsequently undergo rolling circle amplification to generate an amplification product detectable by sequencing or fluorescent readout. In embodiments, the probe set include multiple probes per exon or across multiple exons of the same transcript to increase capture efficiency and improve sensitivity for low-abundance targets. In other embodiments, probes are specifically designed to overlap splice junctions, enabling detection of particular splice isoforms relevant to IBD pathogenesis. For example, detection of isoform-specific transcripts of IL23R, IL6R, or FOXP3 may provide additional clinical utility in stratifying ulcerative colitis versus Crohn's disease.

In embodiments, each of the hybridization sequences of the oligonucleotide probe includes a GC content of about 45% to about 65%. In embodiments, each probe sequence including a first segment and a second segment, each segment including computationally derived characters corresponding to 15 to 20 nucleotides; selecting from the plurality a first subset of probe sequences wherein each segment is complementary to a sequence of the target gene; and selecting, from the first subset, a second subset of probe sequences including: 45% to 65% guanine and/or cytosine nucleotides; no more than four consecutive guanine or cytosine nucleotides; and a predicted melting temperature of greater than 50° C. when forming a duplex with the sequence of the target gene.

The oligonucleotide probes can also be ranked or selected in the methods based on any mathematical combination of two or more nucleotide identities. For example, and not by way of limitation, probes can be ranked and/or selected based on the percentage or fraction of bases that are either guanine or cytosine (“G+C %”) or, alternatively, based on the percentage or fraction of bases that are either adenine or thymine (“A+T”). In another embodiment, oligonucleotide probes can be ranked or selected according to a differential between the percent or fraction of two or more nucleotide identities, such as the difference between the percent or fraction of bases in a probe that are adenine and the percent or fraction of bases that are cytosine (“A<C %”). In embodiments, oligonucleotide probes are ranked and/or selected to retain the number of G and C bases within a preferred range of 40% to 70% of the binding sequence. In particular, it is already well known in the art that guanine-cytosine base pairs have a higher stability than do adenine-thymine base pairs and, further, that many guanine containing mismatches have a higher stability than do non-guanine containing mismatches (see, e.g., SantaLucia, 1998, Proc. Natl. Acad. Sci. U.S.A. 95:1460-1465). As a result, although the percentage of guanine-cytosine base pairs is therefore somewhat correlated with the perfect match duplex binding energy discussed above, high number of guanine-cytosine base pairs are also correlated with higher levels of cross-hybridization. Preferably the percentage of G-C base pairs is between 0 and 75%, more preferably between 0 and 55%, and still more preferably between 8 and 45%.

In embodiments, the method includes removing from the first subset any probe sequence including a region complementary to a sequence in a transcriptome database. In embodiments, a homology search method such as BLAST (“Basic Local Alignment Search Tool”) and PowerBLAST (see, in particular, Altschul et al., 1990, J. Mol. Biol. 215:403-410; Altschul, 1997, Nucleic Acids Res. 25:3389-3402; and Zhang and Madden, 1997, Genome Res. 7:649-656) are performed against each probe sequence to identify polynucleotides, e.g., in a database of expressed sequences such as the GenBank or the dbEST database, which comprises sequences that are most identical or homologous to each probe's complementary sequence. For example, in embodiments, sequences which are at least 50%, 60%, 70%, 80%, 90%, 95%, or 99% identical to a probe's target sequence are identified using a search algorithm such as BLAST or PowerBLAST according to its default parameters. Preferably the search algorithm is employed using parameters set to detect perfect-match sequences of a seed length of, e.g., 7 to 15 or, more preferably, 7 to 12 bases. Binding energies and binding specificity are then evaluated only for polynucleotide sequences identified in such searches. Any probe sequences that have a match identified in a database may then be discarded.

The code probes additionally include a backbone which includes a sequencing primer binding sequence. In this manner, sets of probes may be combined to facilitate multiple rounds of detection. Herein we provide orthogonal sequencing primer sequences which may be included in the backbone of the code probe. For example, a first subset of probes may detect RRM2, VCAM1, S100P, and PROM1 wherein each probe includes the SP1 sequence. Following detection of the first subset, a second subset of probes may be used to detect IL1B, SOX1G, SOX1G, and EPCAM wherein each probe in the second subset includes the SP5 sequence in the backbone. To enable greater detection, the code-foot sequences for the probe targeting ABCG2 (e.g., SEQ ID NO:1 and SEQ ID NO:5710) in the first subset may be different in the second subset (e.g., SEQ ID NO: 17 and SEQ ID NO:5726). In embodiments, the backbone may include a supplemental error corrected barcode (ECB) to further facilitate multiplexing.

SEQUENCING PRIMER TABLE

| Sequencing | Sequencing primer | |
|---|---|---|
| Primer | sequence (5′ to 3′) | |
| SP1 | CCACAGGAAGTAAAGCACA | SEQ ID |
| | CTCTTTCCCTACACGACGC | NO: 10531 |
| | TCTTCCGATC | |
| SP2 | CCACAGGAAGTAAAGCCAC | SEQ ID |
| | AACGGGAGCTGTGGAATTG | NO: 10532 |
| | GTTCACCTGG | |
| SP3 | CCACAGGAAGTAAAGCGTA | SEQ ID |
| | TGATGGTGTTGCGGCTTCT | NO: 10533 |
| | CGCTTAACGC | |
| SP4 | CCACAGGAAGTAAAGCTGT | SEQ ID |
| | TGCATCTCCACCCGGATTG | NO: 10534 |
| | AGCCTTCAGC | |
| SP5 | CCACAGGAAGTAAAGCTGG | SEQ ID |
| | ACTAAGACTCGTCCTCCAG | NO: 10535 |
| | CGGACCTAAG | |
| SP6 | CCACAGGAAGTAAAGCTCG | SEQ ID |
| | GCGTTGTCTGCTATCGTTC | NO: 10536 |
| | TTGGCACTCC | |
| SP7 | ACACTCTTTCCCTACACGA | SEQ ID |
| | CGCTCTTCCGATC | NO: 10537 |
| SP8 | TCGGCGTTGTCTGCTATCG | SEQ ID |
| | TTCTTGGCACTCC | NO: 10538 |
| SP9 | CACAACGGGAGCTGTGGAA | SEQ ID |
| | TTGGTTCACCTGG | NO: 10539 |
| SP10 | TGTTGCATCTCCACCCGGA | SEQ ID |
| | TTGAGCCTTCAGC | NO: 10540 |
| SP11 | TGGACTAAGACTCGTCCTC | SEQ ID |
| | CAGCGGACCTAAG | NO: 10541 |
| SP12 | TACGACACACTCGGGCTCT | SEQ ID |
| | ATGGGCTTCATGG | NO: 10542 |
| SP13 | GTATGATGGTGTTGCGGCT | SEQ ID |
| | TCTCGCTTAACGC | NO: 10543 |
| SP14 | TCTTGAGTCATTCGCAGGG | SEQ ID |
| | CATGTGCCAGACC | NO: 10544 |

Controls. The National Institute of Standards and Technology (NIST) developed the External RNA Controls Consortium (ERCC) to derive universal references. The ERCC assembled a sequence library of 176 DNA sequences that could be transcribed into RNA to serve as controls in systems used to measure gene expression. These controls were cataloged as ERCC-00001 through ERCC-00176, and are collectively referred to as ERCC controls. We designed probes to be specific to some ERCC probes, enabling the detection of false-positive ERCC probes.

What is claimed is:

1. A probe panel comprising a plurality of oligonucleotide probes, wherein each oligonucleotide probe comprises:
a first hybridization sequence designed to specifically bind to a first target sequence of an RNA molecule and a second hybridization sequence designed to specifically bind to a second target sequence of the RNA molecule,
wherein the probe panel is configured to detect at least 25 RNA molecules associated with inflammatory bowel disease (IBD).

2. The probe panel of claim 1, wherein the probe panel is configured to detect a a sequence corresponding to a gene selected from the group consisting of TNF, IL1B, IL6, IL17A, IL10, IL10RA, IL23R, IFNG, CXCL9, CXCL10, CXCL11, CXCR3, CCR7, CD3E, CD4, CD8A, FOXP3, CD68, MS4A1, ICOSLG, MUC2, CLDN1, EPCAM, CEACAM5, KRT20, COL1A1, TGFB1, and VCAM1.

3. The probe panel of claim 1, wherein the probe panel is configured to detect a sequence corresponding to a gene selected from the group consisting of ACKR1, ACKR4, ACSS3, ACTA2, ADIPOQ, ANGPT2, ANKRD29, ANO1, ANXA1, ANXA13, APC, AQP1, AQP3, BEST2, BEST4, BRAF, CA2, CA7, CAMK2N1, CAVIN2, CCK, CCL2, CCL20, CCL3, CCL4, CCL5, CCR2, CCR5, CCR7, CD14, CD163, CD19, CD1C, CD2, CD247, CD27, CD274, CD276, CD28, CD33, CD34, CD36, CD38, CD3D, CD3E, CD4, CD40, CD40LG, CD44, CD47, CD68, CD70, CD74, CD79A, CD80, CD86, CD8A, CDC25C, CDH1, CDH19, CDX2, CEACAM1, CEACAM5, CEACAM8, CENPK, CHGA, CHRM2, CKB, CLCA1, CLCA4, CLDN1, CLDN3, CLDN4, CLEC9A, CLU, CNTNAP2, COL1A1, CPB1, CSF1R, CSPG4, CTLA4, CTNNA2, CTNNB1, CTSS, CX3CL1, CX3CR1, CXCL10, CXCL11, CXCL13, CXCL9, CXCR3, CXCR4, CXCR5, CXCR6, CYP1A1, CYP2A6, DDIT4, DEFA5, DES, DGKG, DPT, DSP, EGFR, ELF3, EOMES, EPCAM, EPHB3, ERBB2, ESM1, FABP1, FAM210B, FAP, FAS, FASN, FBLN1, FBN1, FCAR, FCGR1A, FCGR3A, FGB, FGFR4, FN1, FOXP3, FSCN1, FZD7, GATA3, GATM, GIP, GNLY, GPC1, GPR183, GPRC5A, GPX2, GREM1, GREM2, GRHL1, GUCA2A, GZMA, GZMB, GZMH, GZMK, HAVCR2, HLA-A, HLA-DRA, HNF4A, HOXD8, ICOSLG, ID2, IDO1, IER3, IFNG, IGFBP7, IGHA1, IGHD, IGHG1, IGHM, IL10, IL10RA, IL17A, IL18R1, IL1B, IL2RA, IL2RB, IL6, IL7R, INS, IRF1, ITGAM, ITGAX, ITGB2, ITLN1, JAK3, JCHAIN, KDR, KIT, KLF1, KLRB1, KLRD1, KLRF1, KLRK1, KRAS, KRT20, LAG3, LAMC3, LARS1, LGR5, LPL, LTBP2, LUM, LYVE1, LYZ, MADCAM1, MAPK1, MET, MKI67, MLN, MMP1, MMRN1, MMRN2, MRC1, MS4A1, MUC1, MUC12, MUC2, MUC5B, MUC6, MYC, MYH11, NCAM1, NCR1, NEUROD1, NEUROG3, NKG7, NONO, NOVA1, NRXN1, NTS, ODC1, OLFM4, PAX2, PDCD1, PDCD1LG2, PDE4A, PDGFRA, PDGFRB, PDK4, PECAMI, PHGR1, PIM1, PLAT, PLIN1, PLXND1, POLD2, POLR2A, PON2, POSTN, POU2AF1, POU2F3, PPARG, PRDM1, PRF1, PROM1, PROX1, PTGS2, PYGB, PYY, RARRES1, RBP2, REG1A, REG4, RGMB, RGS5, ROBO1, ROBO2, RRM2, RSPO3, RUNX1, S100A9, S100P, SCGN, SDC1, SELE, SELL, SETD5, SH2D6, SKA3, SLC16A1, SLC2A1, SLC3A2, SLC6A19, SLC7A5, SMOC2, SNCA, SNCG, SOX10, SPINK4, SST, ST14, STAT1, STAT3, STAT4, STC1, SYTL2, TAGLN, TAP1, TAP2, TBX21, TCF7, TCL1A, TFF3, TFP1, TGFB1, THBS1, THY1, TICRR, TIGIT, TLR2, TLR4, TLR9, TNF, TNFRSF17, TNFRSF4, TNFRSF9, TNFSF13B, TNFSF9, TOP2A, TP53, TPH1, TSPAN8, VCAM1, VCAN, VEGFA, VIM, VWF, WARS1, WNT2B, WNT5B, and ZNF800.

4. A method for detecting a nucleic acid molecule in a tissue, said method comprising:
immobilizing a tissue to a solid support, wherein the tissue comprises a nucleic acid molecule and is selected from colon tissue, rectal tissue, ileal tissue, small intestine tissue, gastric tissue, esophageal tissue, perianal tissue, fibrotic tissue, and granulomatous tissue;
contacting the tissue with an oligonucleotide probe, wherein said oligonucleotide probe comprises a first hybridization sequence and a second hybridization sequence;
hybridizing the first hybridization sequence to the nucleic acid molecule and hybridizing the second hybridization sequence to the nucleic acid molecule;
ligating the first hybridization sequence to the second hybridization sequence to form a circular polynucleotide;
amplifying the circular polynucleotide to form an amplification product; and
sequencing a sequence of the amplification product.

5. The method of claim 4, further comprising detecting the nucleic acid molecule by identifying the first hybridization sequence, the second hybridization sequence, or both the first and second hybridization sequences.

6. The method of claim 4, wherein the oligonucleotide probe further comprises a barcode sequence.

7. The method of claim 4, wherein the nucleic acid molecule comprises a gene sequence corresponding to a gene selected from the group consisting of ACKR1, ACKR4, ACSS3, ACTA2, ADIPOQ, ANGPT2, ANKRD29, ANO1, ANXA1, ANXA13, APC, AQP1, AQP3, BEST2, BEST4, BRAF, CA2, CA7, CAMK2N1, CAVIN2, CCK, CCL2, CCL20, CCL3, CCL4, CCL5, CCR2, CCR5, CCR7, CD14, CD163, CD19, CD1C, CD2, CD247, CD27, CD274, CD276, CD28, CD33, CD34, CD36, CD38, CD3D, CD3E, CD4, CD40, CD40LG, CD44, CD47, CD68, CD70, CD74, CD79A, CD80, CD86, CD8A, CDC25C, CDH1, CDH19, CDX2, CEACAM1, CEACAM5, CEACAM8, CENPK, CHGA, CHRM2, CKB, CLCA1, CLCA4, CLDN1, CLDN3, CLDN4, CLEC9A, CLU, CNTNAP2, COL1A1, CPB1, CSF1R, CSPG4, CTLA4, CTNNA2, CTNNB1, CTSS, CX3CL1, CX3CR1, CXCL10, CXCL11, CXCL13, CXCL9, CXCR3, CXCR4, CXCR5, CXCR6, CYP1A1, CYP2A6, DDIT4, DEFA5, DES, DGKG, DPT, DSP, EGFR, ELF3, EOMES, EPCAM, EPHB3, ERBB2, ESM1, FABP1, FAM210B, FAP, FAS, FASN, FBLN1, FBN1, FCAR, FCGR1A, FCGR3A, FGB, FGFR4, FN1, FOXP3, FSCN1, FZD7, GATA3, GA™, GIP, GNLY, GPC1, GPR183, GPRC5A, GPX2, GREM1, GREM2, GRHL1, GUCA2A, GZMA, GZMB, GZMH, GZMK, HAVCR2, HLA-A, HLA-DRA, HNF4A, HOXD8, ICOSLG, ID2, IDO1, IER3, IFNG, IGFBP7, IGHA1, IGHD, IGHG1, IGHM, IL10, IL10RA, IL17A, IL18R1, IL1B, IL2RA, IL2RB, IL6, IL7R, INS, IRF1, ITGAM, ITGAX, ITGB2, ITLN1, JAK3, JCHAIN, KDR, KIT, KLF1, KLRB1, KLRD1, KLRF1, KLRK1, KRAS, KRT20, LAG3, LAMC3, LARS1, LGR5, LPL, LTBP2, LUM, LYVE1, LYZ, MADCAM1, MAPK1, MET, MKI67, MLN, MMP1, MMRN1, MMRN2, MRC1, MS4A1, MUC1, MUC12, MUC2, MUC5B, MUC6, MYC, MYH11, NCAM1, NCR1, NEUROD1, NEUROG3, NKG7, NONO, NOVA1, NRXN1, NTS, ODC1, OLFM4, PAX2, PDCD1, PDCD1LG2, PDE4A, PDGFRA, PDGFRB, PDK4, PECAMI, PHGR1, PIM1, PLAT, PLIN1, PLXND1, POLD2, POLR2A, PON2, POSTN, POU2AF1, POU2F3, PPARG, PRDM1, PRF1, PROM1, PROX1, PTGS2, PYGB, PYY, RARRES1, RBP2, REG1A, REG4, RGMB, RGS5, ROBO1, ROBO2, RRM2, RSPO3, RUNX1, S100A9, S100P, SCGN, SDC1, SELE, SELL, SETD5, SH2D6, SKA3, SLC16A1, SLC2A1, SLC3A2, SLC6A19, SLC7A5, SMOC2, SNCA, SNCG, SOX10, SPINK4, SST, ST14, STAT1, STAT3, STAT4, STC1, SYTL2, TAGLN, TAP1, TAP2, TBX21, TCF7, TCL1A, TFF3, TFP1, TGFB1, THBS1, THY1, TICRR, TIGIT, TLR2, TLR4, TLR9, TNF, TNFRSF17, TNFRSF4, TNFRSF9, TNFSF13B, TNFSF9, TOP2A, TP53, TPH1, TSPAN8, VCAM1, VCAN, VEGFA, VIM, VWF, WARS1, WNT2B, WNT5B, and ZNF800.

8. The method of claim 4, wherein the nucleic acid molecule comprises a gene sequence corresponding to a gene selected from the group consisting of TNF, IL1B, IL6, IL17A, IL10, IL10RA, IL23R, IFNG, CXCL9, CXCL10, CXCL11, CXCR3, CCR7, CD3E, CD4, CD8A, FOXP3, CD68, MS4A1, ICOSLG, MUC2, CLDN1, EPCAM, CEACAM5, KRT20, COL1A1, TGFB1, and VCAM1.

9. The method of claim 4, wherein the amplification product is generated by rolling circle amplification.

10. The method of claim 4, wherein the nucleic acid molecule is an RNA molecule and the oligonucleotide probe is designed to bind an exon of the RNA molecule.

11. The method of claim 4, further comprising binding a specific binding reagent comprising an oligonucleotide to a protein in the tissue, binding a circularizable oligonucleotide to the oligonucleotide, circularizing the circularizable oligonucleotide to form a second circular polynucleotide, amplifying the second circular polynucleotide to form a second amplification product; and detecting the amplification product.

12. The method of claim 11, wherein the specific binding reagent is selected from an antibody, single-chain Fv fragment (scFv), antibody fragment antigen-binding (Fab), or an aptamer.

13. The method of claim 4, further comprising contacting the tissue with a stain.

14. The method of claim 4, further comprising detecting a CDR3 sequence in the tissue, wherein detecting the CDR3 sequence comprises:
(i) contacting the tissue with a polynucleotide probe and hybridizing a first end of the polynucleotide probe to a first sequence of the nucleic acid molecule, and hybridizing a second end of the polynucleotide probe to a second sequence of the nucleic acid molecule, wherein said nucleic acid molecule comprises the CDR3 sequence between the first sequence and the second sequence;
(ii) extending the polynucleotide probe along the CDR3 sequence to generate a complement of the CDR3 sequence, and ligating the complement of the CDR3 sequence to the polynucleotide probe thereby forming a circular oligonucleotide; and
(iii) sequencing the circular oligonucleotide, or a complement thereof.

15. A method of detecting nucleic acid molecules in a tissue sample from a patient having or suspected of having inflammatory bowel disease (IBD), the method comprising:
i) contacting the tissue sample with a first polynucleotide probe and binding said first polynucleotide probe to a first nucleic acid molecule, and contacting the sample with a second polynucleotide probe and binding said second polynucleotide probe to a second nucleic acid molecule, wherein said first polynucleotide probe comprises a first oligonucleotide binding sequence and a first sequence, and wherein said second polynucleotide probe comprises a second oligonucleotide binding sequence and a second sequence;
ii) amplifying the first and second polynucleotide probes to generate amplification products;
iii) hybridizing a first oligonucleotide to the first hybridization sequence and detecting a series of fluorescent signals associated with the first sequence to determine the first nucleic acid molecule and a location of the first nucleic acid molecule, followed by hybridizing a second oligonucleotide to the second oligonucleotide binding sequence, and detecting a series of fluorescent signals associated with the second sequence to determine the second nucleic acid molecule and a location of the second nucleic acid molecule, wherein the first oligonucleotide and the second oligonucleotide comprise different sequences; and
iv) generating an image of the tissue sample comprising indicia data corresponding to the locations of the first and second nucleic acid molecules.

16. The method of claim 15, further comprising identifying the patient as having inflammatory bowel disease (IBD) when the indicia data in the generated image of the tissue sample comprises a spatial pattern of gene expression associated with IBD.

17. A solid support comprising a plurality of discrete tissue samples obtained from a patient having or suspected of having inflammatory bowel disease (IBD),
wherein each tissue sample is selected from colon tissue, rectal tissue, ileal tissue, small intestine tissue, gastric tissue, esophageal tissue, perianal tissue, fibrotic tissue, or granulomatous tissue;
wherein each tissue sample comprises: (i) an RNA molecule hybridized to an oligonucleotide probe, the RNA molecule comprising a sequence of an IBD-associated gene; and (ii) a protein bound to a specific binding reagent, the protein being an IBD-associated protein.

18. The solid support of claim 17, wherein the RNA molecule comprises a sequence of a gene selected from the group consisting of TNF, IL1B, IL6, IL17A, IFNG, IL10, FOXP3, CXCL9, CXCL10, and MUC2.

19. The solid support of claim 17, wherein the RNA molecule and the protein are co-localized within a cell of the tissue sample.

20. The solid support of claim 18, wherein the protein is selected from the group consisting of CD3E, CD4, CD8A, FOXP3, CD68, MS4A1, EPCAM, and PDCD1.
